(19) United States
(12) Patent Application Publication
(10) Pub. No.: US 2021/0145963 A9
(43) Pub. Date: May 20, 2021

(54) E1E2 HCV VACCINES AND METHODS OF USE

(71) Applicant: THE GOVERNORS OF THE UNIVERSITY OF ALBERTA, Edmonton (CA)

(72) Inventors: Michael Houghton, Edmonton (CA); Darren Hockman, Edmonton (CA); John L. Law, Edmonton (CA); Michael Logan, Edmonton (CA); D. Lorne Tyrrell, Edmonton (CA)

(21) Appl. No.: 14/783,699

(22) PCT Filed: May 15, 2014
(86) PCT No.: PCT/IB2014/001972

§ 371 Date: Oct. 9, 2015

Related U.S. Application Data
(60) Provisional application No. 61/823,712, filed on May 15, 2013.
(60) Provisional application No. 61/887,229, filed on Oct. 4, 2013.

(51) Int. Cl. A61K 39/29

(52) U.S. Cl. CPC A61K 39/29

(57) ABSTRACT

The present disclosure provides immunogenic compositions comprising HCV E1, E2, or E1/E2 polypeptides from two or more different HCV genotypes. The present disclosure provides immunogenic compositions comprising HCV E2 or E1/E2 polypeptides from two or more different HCV genotypes. The immunogenic compositions are useful in carrying out methods of inducing an immune response to HCV. The present disclosure further provides methods of stimulating an immune response to HCV in an individual.

### CROSS-REFERENCE

This application claims the benefit of U.S. Provisional Patent Application Nos. 61/823,712, filed May 15, 2013, and 61/887,229, filed Oct. 4, 2013, which applications are incorporated herein by reference in their entirety.

### INCORPORATION BY REFERENCE OF SEQUENCE LISTING PROVIDED AS A TEXT FILE

A Sequence Listing is provided herewith as a text file, “UALB-017WO SeqList_ST25.txt” created on May 9, 2014 and having a size of 339 KB. The contents of the text file are incorporated by reference herein in their entirety.

### INTRODUCTION

Hepatitis C virus (HCV) is a blood-borne pathogen that is estimated to infect 150-200 million people worldwide. Infection by HCV may be non-symptomatic, and can be cleared by patients, sometimes without medical intervention. However, the majority of patients develop a chronic HCV infection, which may lead to liver inflammation, scarring, and even to liver failure or liver cancer. In the United States alone, over 3 million people have a chronic infection.

The HCV virion contains a positive-sense single stranded RNA genome of about 9.5 kb. The genome encodes a single polyprotein of 3,010 to 3,030 amino acids. The structural proteins comprise a core protein forming the viral nucleocapsid and two envelope glycoproteins, E1 and E2.

There is a need in the art for immunogenic compositions that induce an immune response to HCV in an individual so as to provide induction of an immune response effective against multiple HCV genotypes found around the world, e.g., genotypes 1 and 3.

### SUMMARY

The present disclosure provides immunogenic compositions comprising HCV E1, E2, or E1/E2 polypeptides from two or more different HCV genotypes. The present disclosure provides immunogenic compositions comprising HCV E2 or E1/E2 polypeptides from two or more different HCV genotypes. The immunogenic compositions are useful in carrying out methods of inducing an immune response to HCV. The present disclosure further provides methods of stimulating an immune response to HCV in an individual.

In some embodiments, the present disclosure provides an immunogenic composition comprising: a) an hepatitis C virus (HCV) E1 polypeptide, E2 polypeptide or E1E2 polypeptide from a first HCV genotype; b) an HCV E1 polypeptide, E2 polypeptide, or E1E2 polypeptide from a second HCV genotype; and c) a pharmaceutically acceptable excipient, with the proviso that the composition comprises at least one E1 polypeptide and at least one E2 polypeptide. In some of these embodiments, the first HCV genotype is genotype 1; and the second HCV genotype is genotype 2. In other cases, the first HCV genotype is genotype 1; and the second HCV genotype is genotype 3. The following embodiments i) through xiv) are non-limiting examples, where a subject immunogenic composition comprises:

i) an E1/E2 heterodimeric polypeptide complex of HCV genotype 1; and E1/E2 polypeptide of HCV genotype 3;

ii) an E1/E2 heterodimeric polypeptide complex of HCV genotype 1; and an E2 polypeptide of HCV genotype 3;

iii) an E2 polypeptide of HCV genotype 1; and an E1/E2 heterodimeric polypeptide complex of HCV genotype 3;

iv) an E1/E2 heterodimeric polypeptide complex of HCV genotype 1; and an E1/E2 heterodimeric polypeptide complex of HCV genotype 2;

v) an E1/E2 heterodimeric polypeptide complex of HCV genotype 1; and an E2 polypeptide of HCV genotype 2;

vi) an E2 polypeptide of HCV genotype 1; and an E1/E2 heterodimeric polypeptide complex of HCV genotype 2;

vii) an E1 polypeptide of HCV genotype 1; and an E2 polypeptide of HCV genotype 3;

viii) an E1 polypeptide of HCV genotype 1; and an E1/E2 heterodimeric polypeptide complex of HCV genotype 3;

ix) an E2 polypeptide of HCV genotype 1; and an E1 polypeptide of HCV genotype 3;

x) an E2 polypeptide of HCV genotype 1; and an E1/E2 heterodimeric polypeptide complex of HCV genotype 3;

xi) an E1 polypeptide of HCV genotype 1; and an E2 polypeptide of HCV genotype 2;

xii) an E1 polypeptide of HCV genotype 1; and an E1/E2 heterodimeric polypeptide complex of HCV genotype 2;

xiii) an E2 polypeptide of HCV genotype 1; and an E1 polypeptide of HCV genotype 2; or

xiv) an E2 polypeptide of HCV genotype 1; and an E1/E2 heterodimeric polypeptide complex of HCV genotype 2.

In any of the above-described embodiments, the composition can further include an HCV E1, E2, or E1/E2 polypeptide of a third HCV genotype.

In any of the above-described embodiments, the HCV E2 polypeptide from the first HCV genotype can be wild-type, and the HCV E2 polypeptide from the second HCV genotype can be wild-type. In any of the above-described embodiments, the HCV E2 polypeptide from the first HCV genotype can be wild-type, and the HCV E2 polypeptide from the second HCV genotype can comprise an amino acid substitution of asparagine in an E2N1 site and/or an E2N6 site. In any of the above-described embodiments, the HCV E2 polypeptide from the first HCV genotype can comprise an amino acid substitution of asparagine in an E2N1 site and/or an E2N6 site, and the HCV E2 polypeptide from the second HCV genotype can be wild-type.

In a subject immunogenic composition, the pharmaceutically acceptable excipient can comprise an adjuvant; in some cases, the adjuvant is MF59, alum, poly(DL-lactide co-glycolide), or a CpG oligonucleotide.

In other embodiments, the present disclosure provides an immunogenic composition comprising: a) a hepatitis C virus (HCV) E1 polypeptide, E2 polypeptide, or E1/E2 polypeptide from a first HCV genotype; b) an HCV E1 polypeptide, E2 polypeptide, or E1/E2 polypeptide from a second HCV genotype; c) an HCV E1 polypeptide, E2 polypeptide, or E1/E2 polypeptide from a third HCV genotype; and d) a pharmaceutically acceptable excipient, with the proviso that the composition comprises at least one E1 polypeptide and at least one E2 polypeptide. In some cases, the first HCV genotype is genotype 1, the second HCV genotype is genotype 2, and the third HCV genotype is genotype 3. The following embodiments i) through xvii) are non-limiting examples, where a subject immunogenic composition comprises:

i) an E1/E2 heterodimeric polypeptide complex of HCV genotype 1; an E1/E2 heterodimeric polypeptide complex of HCV genotype 2; and an E1/E2 heterodimeric polypeptide complex of HCV genotype 3;

ii) an E1/E2 heterodimeric polypeptide complex of HCV genotype 1; an E1/E2 heterodimeric polypeptide complex of HCV genotype 2; and an E2 polypeptide of HCV genotype 3;

iii) an E1/E2 heterodimeric polypeptide complex of HCV genotype 1; an E2 polypeptide of HCV genotype 2; and an E1/E2 heterodimeric polypeptide complex of HCV genotype 3;

iv) an E2 polypeptide of HCV genotype 1; an E1/E2 heterodimeric polypeptide complex of HCV genotype 2; and an E1/E2 heterodimeric polypeptide complex of HCV genotype 3;

v) an E1/E2 heterodimeric polypeptide complex of HCV genotype 1; an E2 polypeptide of HCV genotype 2; and an E2 polypeptide of HCV genotype 3;

vi) an E2 polypeptide of HCV genotype 1; an E1/E2 heterodimeric polypeptide complex of HCV genotype 2; and an E2 polypeptide of HCV genotype 3;

vii) an E2 polypeptide of HCV genotype 1; an E2 polypeptide of HCV genotype 2; and an E1/E2 heterodimeric polypeptide complex of HCV genotype 3;

viii) an E1 polypeptide of HCV genotype 1; an E1/E2 heterodimeric polypeptide complex of HCV genotype 2; and an E1/E2 heterodimeric polypeptide complex of HCV genotype 3;

ix) an E1/E2 heterodimeric polypeptide complex of HCV genotype 1; an E1 polypeptide of HCV genotype 2; and an E1/E2 heterodimeric polypeptide complex of HCV genotype 3;

x) an E1/E2 heterodimeric polypeptide complex of HCV genotype 1; an E1/E2 heterodimeric polypeptide complex of HCV genotype 2; and an E1 polypeptide of HCV genotype 3;

xi) an E1 polypeptide of HCV genotype 1; an E2 polypeptide of HCV genotype 2; and an E1/E2 heterodimeric polypeptide complex of HCV genotype 3;

xii) an E2 polypeptide of HCV genotype 1; an E1 polypeptide of HCV genotype 2; and an E1/E2 heterodimeric polypeptide complex of HCV genotype 3;

xiii) an E1/E2 heterodimeric polypeptide complex of HCV genotype 1; an E2 polypeptide of HCV genotype 2; and an E1 polypeptide of HCV genotype 3;

xiv) an E1 polypeptide of HCV genotype 1; an E2 polypeptide of HCV genotype 2; and an E2 polypeptide of HCV genotype 3;

xv) an E2 polypeptide of HCV genotype 1; an E1 polypeptide of HCV genotype 2; and an E2 polypeptide of HCV genotype 3;

xvi) an E2 polypeptide of HCV genotype 1; an E2 polypeptide of HCV genotype 2; and an E1 polypeptide of HCV genotype 3; or

xvii) an E1 polypeptide of HCV genotype 1; an E1/E2 heterodimeric polypeptide complex of HCV genotype 2; and an E2 polypeptide of HCV genotype 3.

In any of the above-mentioned embodiments, the HCV E2 polypeptide from the first HCV genotype can be wild-type, the HCV E2 polypeptide from the second HCV genotype can be wild-type, and the HCV E2 polypeptide from the third HCV genotype can be wild-type. In other cases, the HCV E2 polypeptide from the first HCV genotype is wild-type, the HCV E2 polypeptide from the second HCV genotype is wild-type, and the HCV E2 polypeptide from the third HCV genotype comprises an amino acid substitution of asparagine in an E2N1 site and/or an E2N6 site. In other cases, the HCV E2 polypeptide from the first HCV genotype is wild-type, the HCV E2 polypeptide from the second HCV genotype comprises an amino acid substitution of asparagine in an E2N1 site and/or an E2N6 site, and the HCV E2 polypeptide from the third HCV genotype is wild-type. In other cases, the HCV E2 polypeptide from the first HCV genotype comprises an amino acid substitution of asparagine in an E2N1 site and/or an E2N6 site, the HCV E2 polypeptide from the second HCV genotype is wild-type, and the HCV E2 polypeptide from the third HCV genotype is wild-type.

In any of the above-mentioned embodiments, the pharmaceutically acceptable excipient can include an adjuvant. In any of the above-mentioned embodiments, the adjuvant can be MF59, alum, poly(DL-lactide co-glycolide), or a CpG oligonucleotide.

The present disclosure provides a method of inducing an immune response in an individual, the method comprising administering to the individual an effective amount of an immunogenic composition according to any of the embodiments described herein.

### BRIEF DESCRIPTION OF THE DRAWINGS

FIGS. 1A-C provide a protein alignment of examples of the core-E1-E2 coding regions of a HCV genotype 1 virus, specifically representative HCV 1A, 1B and 1C genotypes. Genbank database sequences for the coding region core-E1-E2 were aligned using Geneious software v5.6.4. Glycosylation sites N417 (E2N1) and N532 (E2N6) are highlighted (arrows). Numbering of amino acids is according to strain NP_671941 (H77). To maintain amino acid numbering, strains AF139594 and BAA03581 have an amino acid insertion between amino acid 478 and 479 (lysine (K) and alanine (A), respectively) that is not shown (hashed line). From top to bottom SEQ ID NOs:1-27.

FIGS. 2A and 2B provide a protein alignment of the core-E1-E2 coding region for representative HCV 3A, 3B and 3K genotypes. Genbank database sequences for the coding region core-E1-E2 were aligned using Geneious software v5.6.4. Glycosylation sites N417 (E2N1) and N532 (E2N6) are highlighted (arrows). From top to bottom SEQ ID NOs:28-37.

FIG. 3 provides a consensus E1E2 protein sequence for Alberta HCV genotype 1A isolate (LKS 1). A consensus sequence for the E1E2 coding region was obtained from a patient with HCV genotype 1A (LKS 1) (Li Ka Shing Institute of Virology, University of Alberta, Edmonton, AB, Canada). LKS1 E1E2 is shown in comparison to H77 genotype 1A (core-E1-E2). LKS1 has 515/555 (92.8%) amino acid identities with H77 in the E1E2 coding region. Glycosylation sites N417 (E2N1) and N532 (E2N6) are highlighted (arrows). From top to bottom SEQ ID NO:38, SEQ ID NO:1, and SEQ ID NO:39.

FIG. 4 depicts genotype-specific neutralization by sera of goats immunized with either HCV1 (genotype 1a)-derived E1E2, or with J6 (genotype 2)-derived E2.

FIG. 5 depicts neutralization activity of sera from goats immunized against Genotype 1a chimeric H77c/JFH1 HCV.

FIG. 6 depicts neutralization activity of sera from goats immunized against Genotype 2a chimeric J6/JFH1 HCV.

FIGS. 7A and 7B depict genotype-specific neutralization and cross-neutralization of HCV by sera of goats immunized with HCV antigens. A) Goats 757 and 714 were immunized with recombinant E1E2 derived from genotype 1a HCV1 sequence. B) Goats 766 and 773 were immunized with E2 derived from genotype 2a J6 sequence. Neutralization activity against chimeric HCVcc of genotype 1a-6a was measured. Results were normalized using neutralization activity of sera prior to immunization as control.

FIGS. 8A and 8B provide an amino acid sequence of the core-E1-E2 coding region for HCV genotype 7a. Amino acid sequence for the coding region core-E1-E2 of genotype 7a (isolate QC69; Genbank: ABN05226.1) is shown according to the numbering scheme of the reference strain, NP_671941 (H77). Glycosylation sites N417 (E2N1) and N533 (E2N6) are highlighted (arrows) (SEQ ID NO:40).

FIGS. 9A-C provide an alignment of amino acid sequences of the core-E1-E2 coding region of representative HCV 2A and HCV2B subtypes. Genbank database sequences for the coding region core-E1-E2 were aligned using Geneious software v5.6.4. The amino acid numbering depicted is in accordance to the common HCV strains: ABO47639 (JFH1) and HPCJ8G-J8 (J8) for HCV2A and HCV2B, respectively. From top to bottom SEQ ID NOs:41-53.

### DEFINITIONS

The term “hepatitis C virus” (“HCV”), as used herein, refers to any one of a number of different genotypes and isolates of hepatitis C virus. Thus, “HCV” encompasses any of a number of genotypes, subtypes, or quasispecies, of HCV, including, e.g., genotype 1, 2, 3, 4, 6, 7, etc. and subtypes (e.g., 1a, 1b, 2a, 2b, 3a, 4a, 4c, etc.), and quasispecies. Representative HCV genotypes and isolates include: the “Chiron” isolate HCV-1, H77, J6, Con1, isolate 1, BK, EC1, EC10, HC-J2, HC-J5; HC-J6, HC-J7, HC-J8, HC-JT, HCT18, HCT27, HCV-476, HCV-KF, “Hunan”, “Japanese”, “Taiwan”, TH, type 1, type 1a, H77 type 1b, type 1c, type 1d, type 1e, type 1f, type 10, type 2, type 2a, type 2b, type 2c, type 2d, type 2f, type 3, type 3a, type 3b, type 3g, type 4, type 4a, type 4c, type 4d, type 4f, type 4h, type 4k, type 5, type 5a, type 6 and type 6a.

The terms “individual,” “host,” “subject,” and “patient” are used interchangeably herein, and refer to a mammal, including, but not limited to, non-human primates (e.g., simians), and humans.

As used herein, the term “isolated,” in reference to a polypeptide, refers to a polypeptide that is in an environment different from that in which the polypeptide naturally occurs. An isolated polypeptide can be purified. By “purified” is meant a compound of interest (e.g., a polypeptide) has been separated from components that accompany it in nature. “Purified” can also be used to refer to a polypeptide separated from components that can accompany it during production of the polypeptide (e.g., during synthesis in vitro, etc.). In some embodiments, a polypeptide (or a mixture of polypeptides) is substantially pure when the polypeptide (or mixture of polypeptides) is at least 60% or at least 75% by weight free from organic molecules with which it is naturally associated or with which it is associated during production. In some embodiments, the polypeptide is from 30% to 60% pure. In some embodiments, the polypeptide (or mixture of polypeptides) is at least 60%, at least 75%, at least 80%, at least 85%, at least 90%, at least 95%, or at least 99%, by weight, pure. For example, in some embodiments, an E1 or an E2 polypeptide (or a mixture of E1 and E2 polypeptides) is substantially pure when the E1 or E2 polypeptide (or mixture of E1 and E2 polypeptides) is at least 60% or at least 75% by weight free from organic molecules with which the polypeptide(s) is naturally associated or with which it is associated during production. In some embodiments, the E1 or E2 polypeptide (or mixture of E1 and E2 polypeptides) is at least 60%, at least 75%, at least 80%, at least 85%, at least 90%, at least 95%, or at least 99%, by weight, pure. In some embodiments, where a composition comprises an E2 polypeptide, the E2 polypeptide is at least 60%, at least 75%, at least 80%, at least 85%, at least 90%, at least 95%, or at least 99%, by weight, pure. In some embodiments, where a composition comprises an E1/E2 heterodimeric complex polypeptide, the E1/E2 heterodimeric complex polypeptide is at least 60%, at least 75%, at least 80%, at least 85%, at least 90%, at least 95%, or at least 99%, by weight, pure. In some embodiments, where a composition comprises an E2 polypeptide and an E1/E2 heterodimeric complex polypeptide, the E2 polypeptide and the E1/E2 heterodimeric complex polypeptide are at least 60%, at least 75%, at least 80%, at least 85%, at least 90%, at least 95%, or at least 99%, by weight, pure.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an HCV E1 polypeptide” includes a plurality of such polypeptide and reference to “the HCV genotype” includes reference to one or more genotypes and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides immunogenic compositions comprising HCV E1, E2, or E1/E2 polypeptides from two or more different HCV genotypes. The present disclosure provides immunogenic compositions comprising HCV E2 or E1/E2 polypeptides from two or more different HCV genotypes. The immunogenic compositions are useful in carrying out methods of inducing an immune response to HCV. The present disclosure further provides methods of stimulating an immune response to HCV in an individual.

### Immunogenic Compositions

The present disclosure provides immunogenic compositions comprising HCV structural polypeptides, e.g., E1, E2, and/or E1/E2, where the composition comprises HCV structural polypeptides from at least two different HCV genotypes. The present disclosure provides immunogenic compositions comprising HCV E2 or E1/E2 polypeptides from two or more different HCV genotypes.

A subject immunogenic composition includes HCV polypeptides E1, E2, and/or E1/E2. E1 and E2 polypeptides present in a subject immunogenic composition may be present in the composition as a covalently or non-covalently linked heterodimer. The E1 and E2 polypeptides can be present in the composition as a single polypeptide chain, or can be present as two separate polypeptide chains (which may or may not be covalently linked via a disulfide bond).

The E1 and E2 polypeptides are isolated, and can be purified. In some cases, a subject immunogenic composition comprises E1 and E2 polypeptides, where the polypeptides (or mixtures of E1 and E2 polypeptides) are from 30% to 60% pure, or from 60% to about 80% pure. In some cases, a subject immunogenic composition comprises E1 and E2 polypeptides, where the polypeptides (or mixtures of E1 and E2 polypeptides) are at least about 80% pure, at least about 85% pure, at least about 90% pure, at least about 95% pure, at least about 98% pure, at least about 99% pure, or greater than 99% pure. In some embodiments, a subject immunogenic composition does not include any other polypeptides (e.g. no other HCV polypeptides) other than HCV E1 and HCV E2 polypeptides.

The terms “E1 polypeptide” and “E2 polypeptide” encompass proteins which include additional modifications to the native sequence, such as deletions (e.g., C-terminal truncations, e.g., C-terminally truncated E2 lacking of the naturally-occurring C terminus as described herein), additions and substitutions (e.g., conservative amino acid substitutions). These modifications may be deliberate, as through site-directed mutagenesis, or may occur as a result of mutational events during naturally-occurring or recombinant expression of E1 or E2 (e.g., in vivo in the course of HCV infection, during HCV virus production in cell culture, during recombinant expression or E1 or E2 in in vitro cell culture.

### E1

An HCV E1 polypeptide suitable for use in an immunogenic composition of the present disclosure can have a length of from about 150 amino acids (aa) to about 175 aa, from about 175 aa to about 195 aa, from about 131 aa to about 175 aa, or from about 175 aa to about 193 aa.

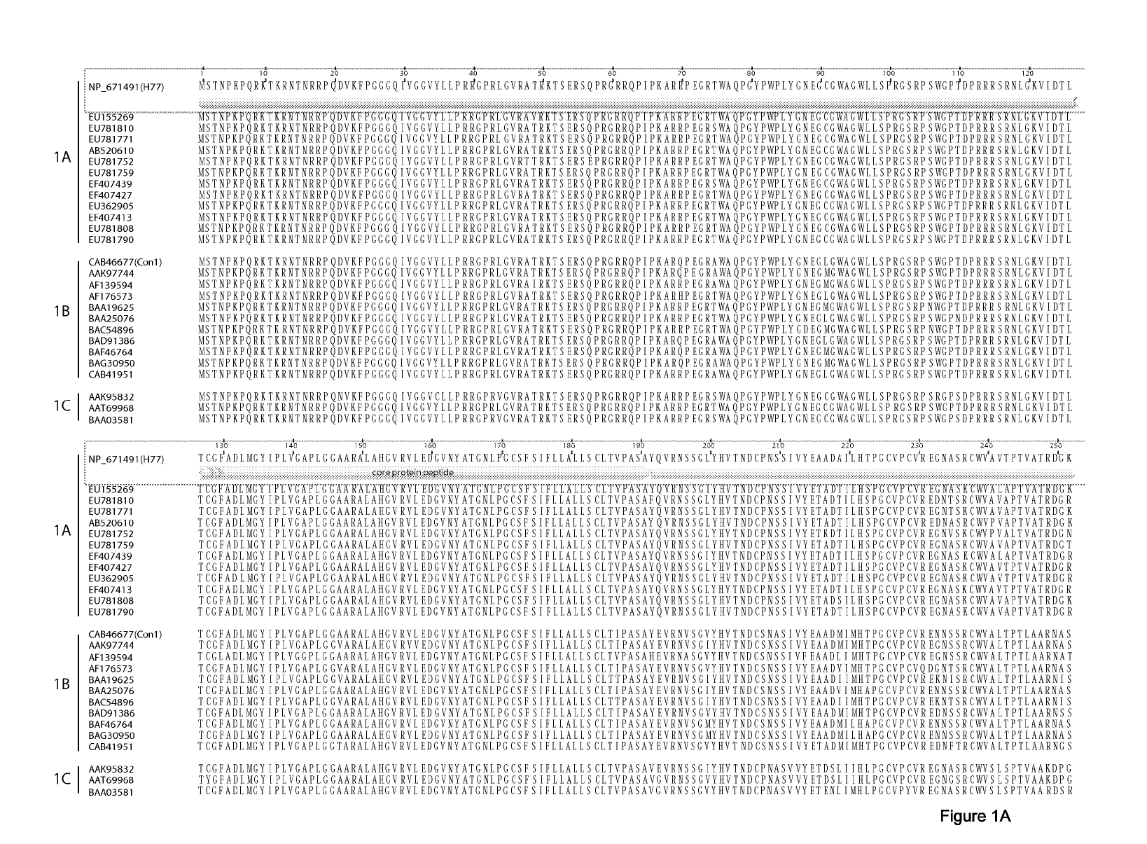

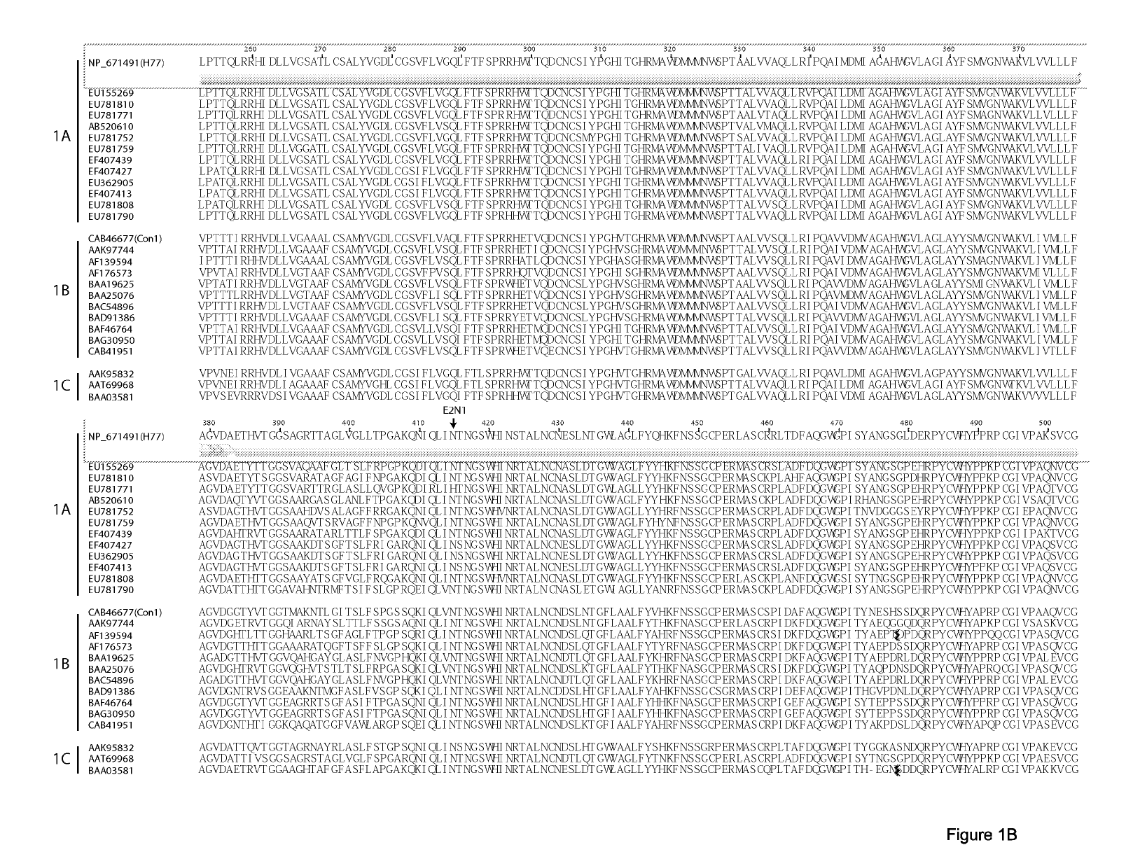

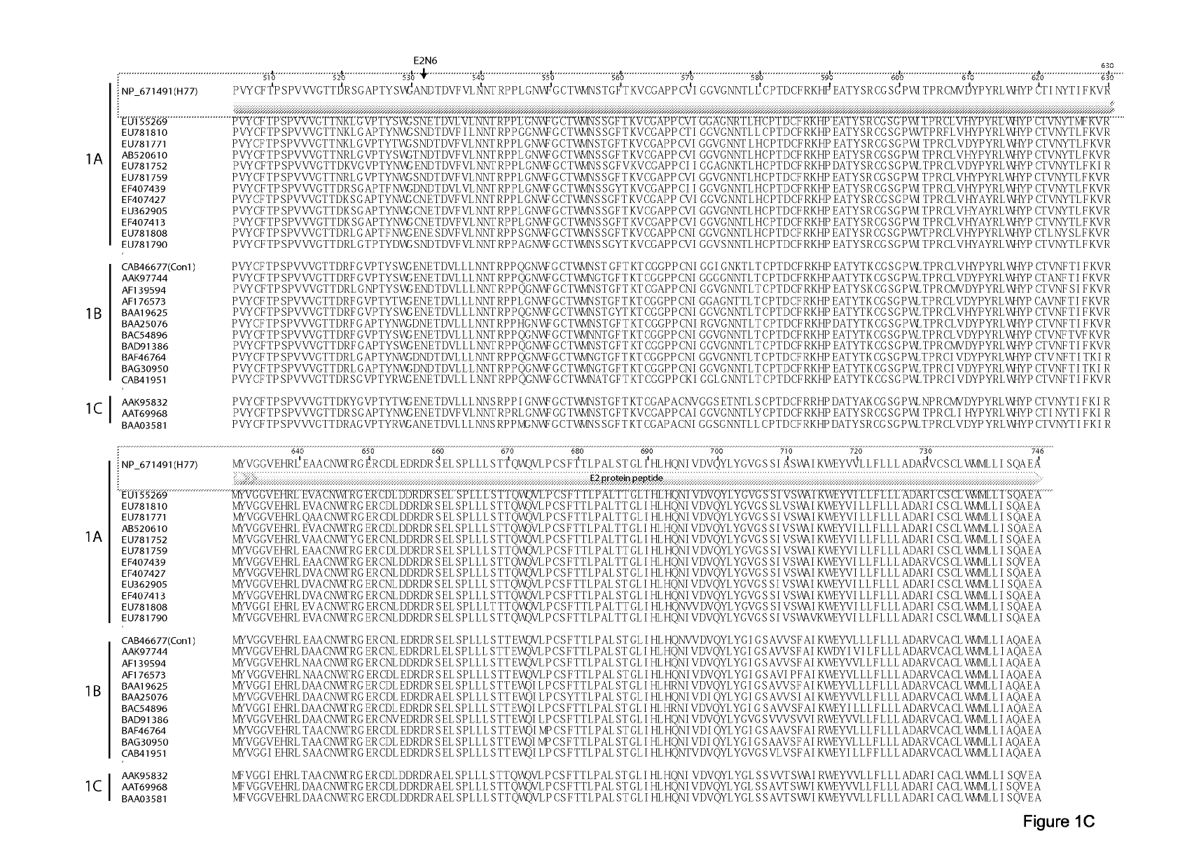

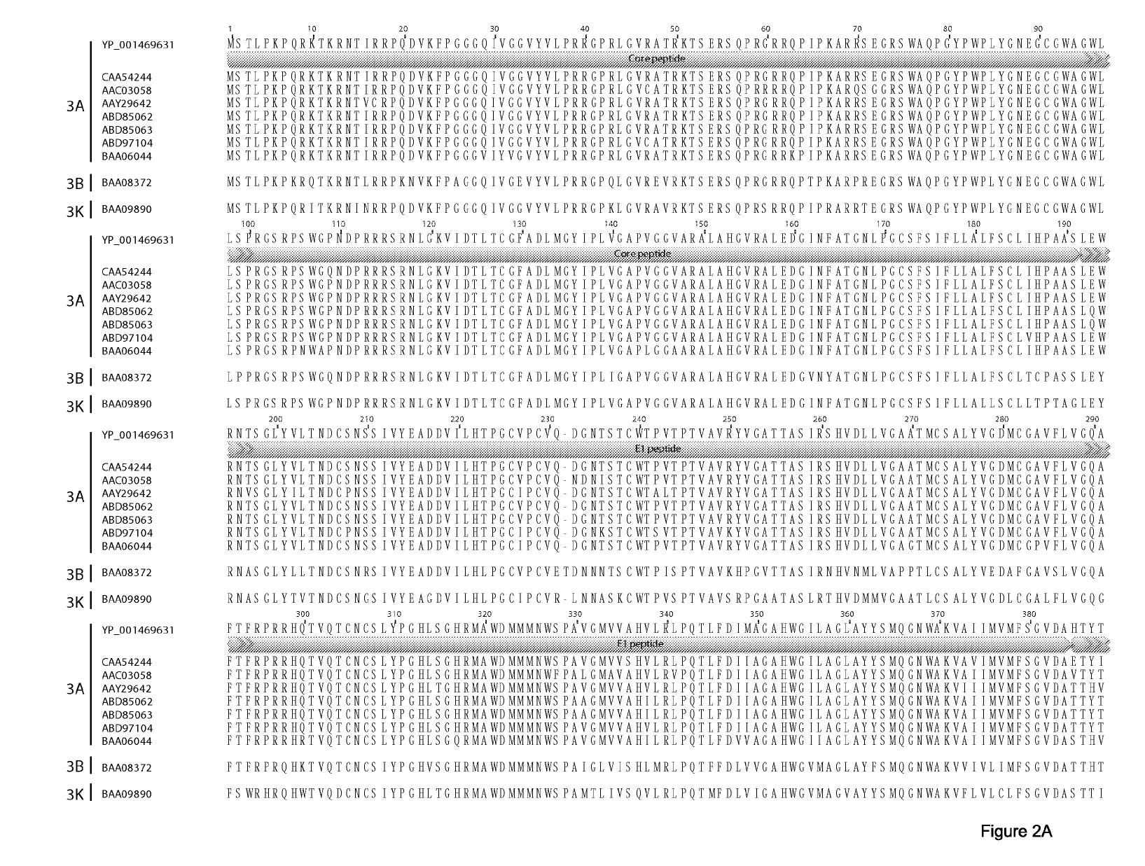

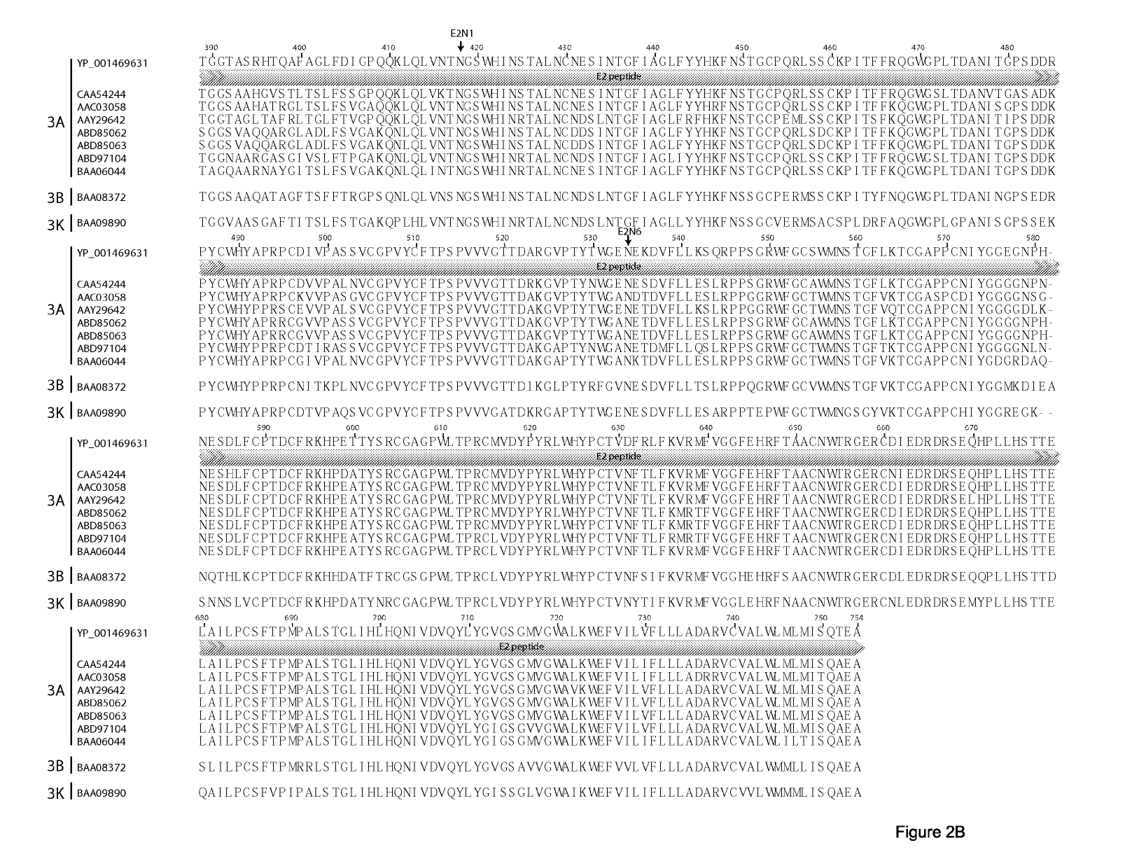

In FIGS. 1A-C, the amino acid sequence of E1 is amino acid 192 to amino acid 383. In FIGS. 2A-2B, the amino acid sequence of E1 is amino acid 192 to amino acid 384. In FIG. 3, the amino acid sequence of E1 is amino acid 192 to amino acid 385. Amino acids at around 170 through approximately 191 serve as a signal sequence for E1. As used herein, “E1 polypeptide” includes a precursor E1 protein, including the signal sequence; includes a mature E1 polypeptide which lacks this sequence; and includes an E1 polypeptide with a heterologous signal sequence. An E1 polypeptide can include a C-terminal membrane anchor sequence which occurs at approximately amino acid positions 360-383 (see, e.g., WO 96/04301). In some cases, a suitable E1 polypeptide lacks a C-terminal portion that includes a transmembrane region. For example, in some cases, a suitable E1 polypeptide lacks the C-terminal portion from amino acid 330 to amino acid 384, or from amino acid 360 to amino acid 384. E1 polypeptides can be an E1 polypeptide of any genotype, subtype or isolate of HCV. E1 polypeptides of genotype 1 and E1 polypeptides of genotype 3 are of particular interest.

An E1 polypeptide can comprise an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIGS. 1A-C, FIGS. 2A-2B, or FIG. 3.

An E1 polypeptide can comprise an amino acid sequence having at least about 70%, at least about 75%, at least about 80%, at least about 85%, at least about 90%, at least about 95%, at least about 98%, at least about 99%, or 100%, amino acid sequence identity to the consensus E1 polypeptide amino acid sequence depicted in FIG. 3.

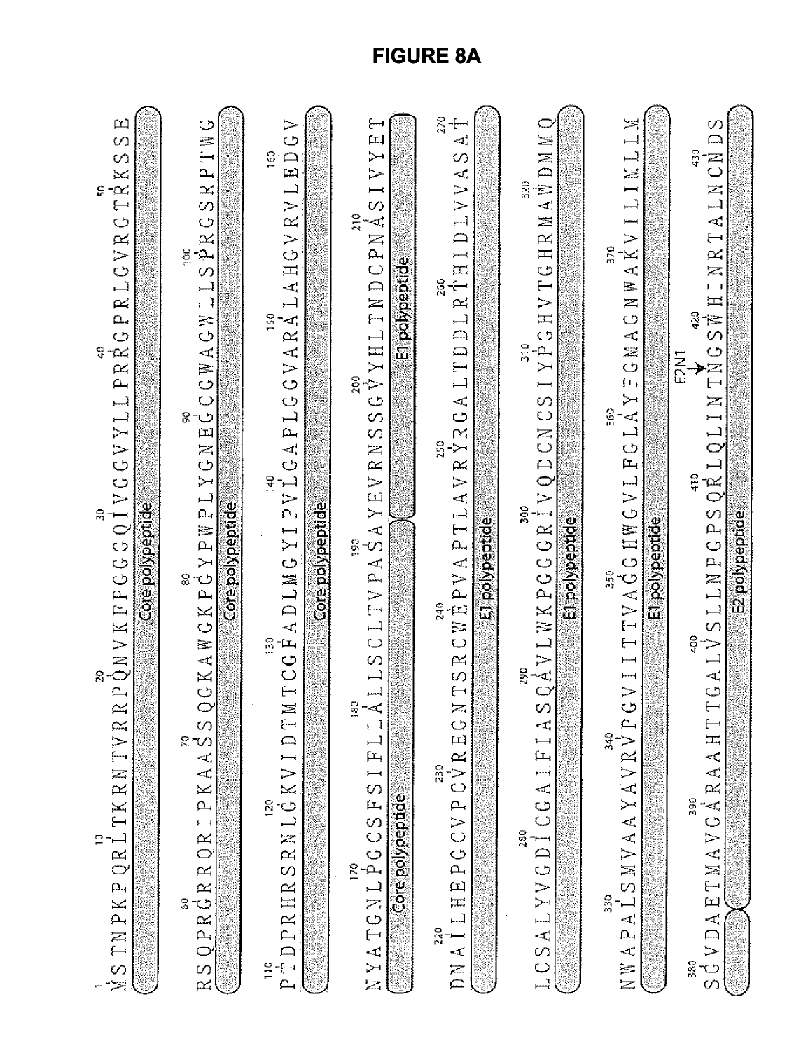

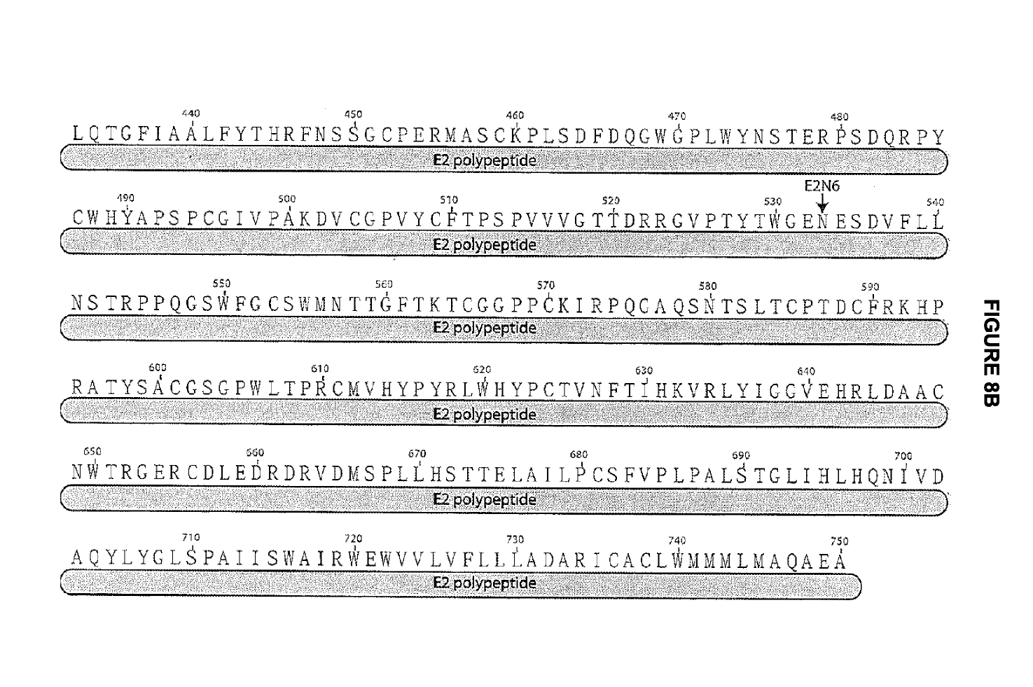

An E1 polypeptide can comprise an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIGS. 8A and 8B. For example, an E1 polypeptide of genotype 7A can comprise an amino acid sequence having at least about 70%, at least about 75%, at least about 80%, at least about 85%, at least about 90%, at least about 95%, at least about 98%, at least about 99%, or 100%, amino acid sequence identity to amino acids 192-383 of the amino acid sequence depicted in FIGS. 8A and 8B.

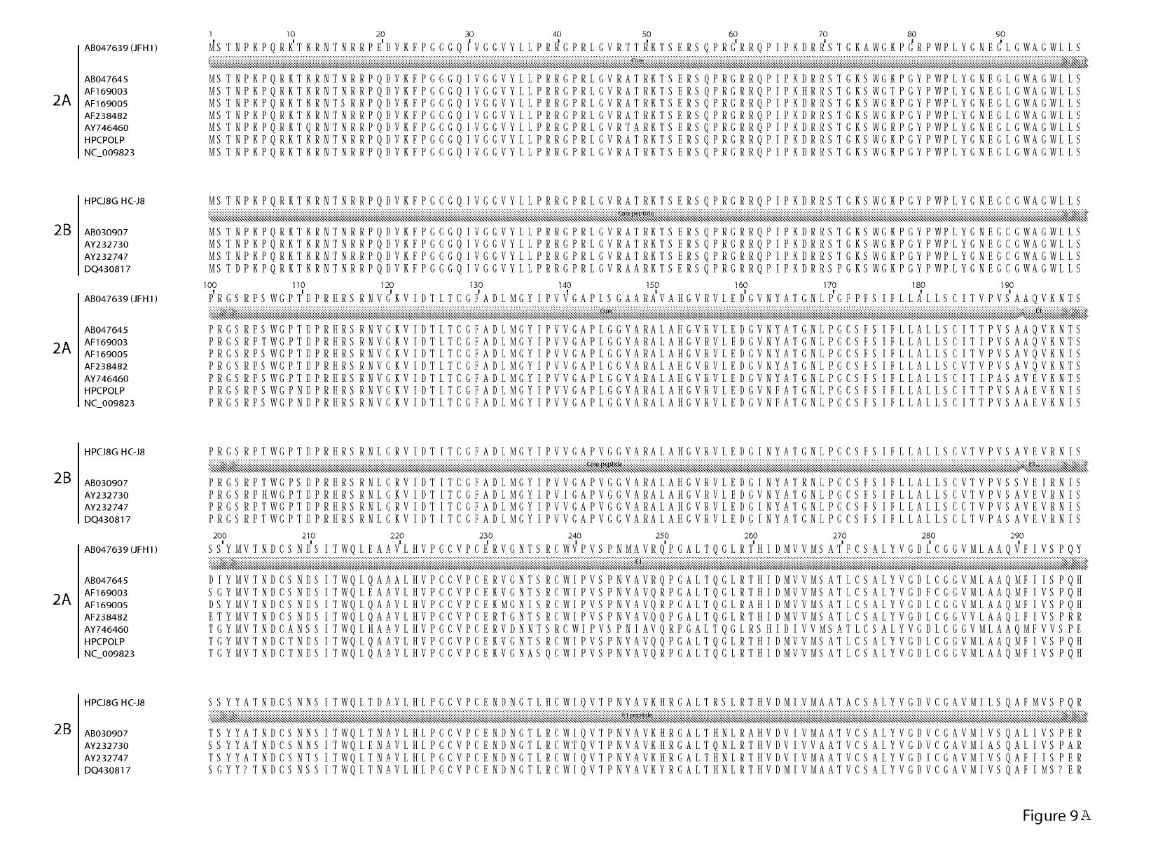

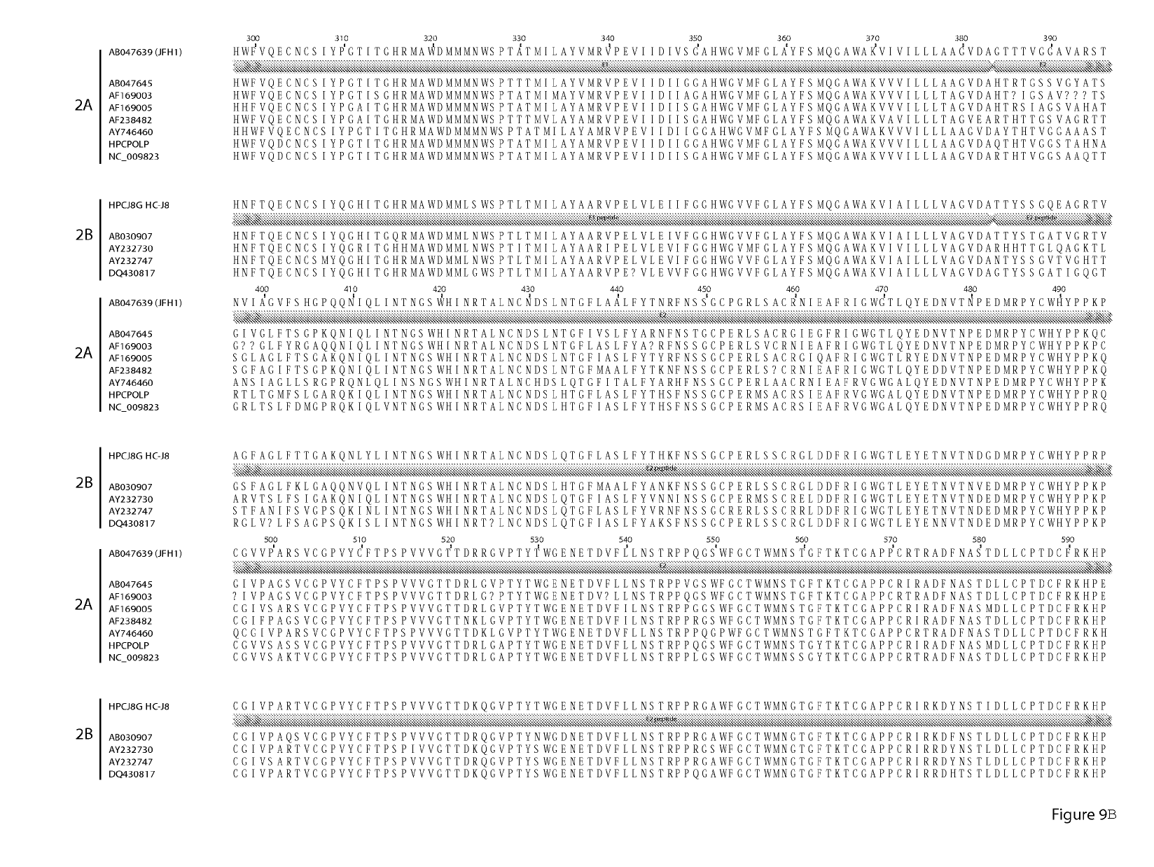

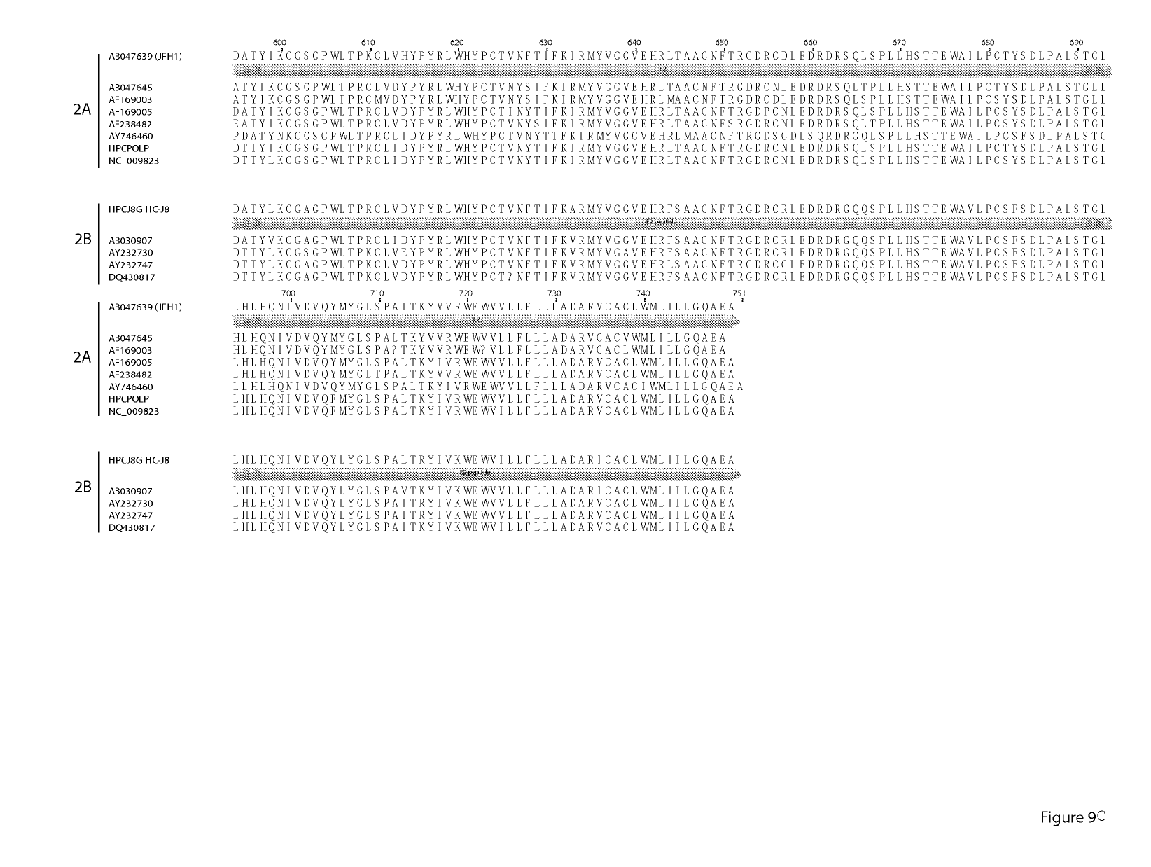

An E1 polypeptide can comprise an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIGS. 9A-C. For example, an E1 polypeptide of genotype 2A can comprise an amino acid sequence having at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 2A and depicted in FIGS. 9A-C. For example, an E1 polypeptide of genotype 2B can comprise an amino acid sequence having at least about 70%, at least about 75%, at least about 80%, at least about 85%, at least about 90%, at least about 95%, at least about 98%, at least about 99%, or 100%, amino acid sequence identity to amino acids 384-751 of an amino acid sequence identified as 2B and depicted in FIGS. 9A-C.

### E2

An E2 polypeptide suitable for use in a subject immunogenic composition can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa.

In FIGS. 1A-C, the amino acid sequence of E2 is amino acid 384 to amino acid 746. In FIGS. 2A-2B, the amino acid sequence of E2 is amino acid 385 to amino acid 754. In FIG. 3, the amino acid sequence of E2 is amino acid 386 to amino acid 746. As used herein, an “E2 polypeptide” includes a precursor E2 protein, including the signal sequence; includes a mature E2 polypeptide which lacks this sequence; and includes an E2 polypeptide with a heterologous signal sequence. An E2 polypeptide can include a C-terminal membrane anchor sequence which occurs at approximately amino acid positions 715-730 and may extend as far as approximately amino acid residue 746 (see, Lin et al., J. Virol. (1994) 68:5063-5073).

In some cases, a E2 polypeptide suitable for use in an immunogenic composition of the present disclosure lacks a portion of its C-terminal region, e.g., from about amino acid 715 to the C-terminus; from about amino acid 625 to the C-terminus; from about amino acid 661 to the C-terminus; from about amino acid 655 to the C-terminus; from about amino acid 500 to the C-terminus, where the amino acid numbering is with reference to the numbering in FIGS. 1A-C. See, e.g., U.S. Pat. No. 6,521,423.

An E2 polypeptide suitable for use in an immunogenic composition of the present disclosure can comprise an amino acid sequence having at least about 70%, at least about 75%, at least about 80%, at least about 85%, at least about 90%, at least about 95%, at least about 98%, at least about 99%, or 100%, amino acid sequence identity to the amino acid sequence of an E2 polypeptide depicted in FIGS. 1A-C, FIGS. 2A-2B, or FIG. 3.

An E2 polypeptide suitable for use in an immunogenic composition of the present disclosure can comprise an amino acid sequence having at least about 70%, at least about 75%, at least about 80%, at least about 85%, at least about 90%, at least about 95%, at least about 98%, at least about 99%, or 100%, amino acid sequence identity to the amino acid sequence of an E2 polypeptide depicted in FIGS. 8A and 8B. For example, an E2 polypeptide of genotype 7A can comprise an amino acid sequence having at least about 70%, at least about 75%, at least about 80%, at least about 85%, at least about 90%, at least about 95%, at least about 98%, at least about 99%, or 100%, amino acid sequence identity to amino acids 384-750 of the amino acid sequence depicted in FIGS. 8A and 8B.

An E2 polypeptide suitable for use in an immunogenic composition of the present disclosure can comprise an amino acid sequence having at least about 70%, at least about 75%, at least about 80%, at least about 85%, at least about 90%, at least about 95%, at least about 98%, at least about 99%, or 100%, amino acid sequence identity to the amino acid sequence of an E2 polypeptide depicted in FIGS. 9A-C. For example, an E2 polypeptide of genotype 2A can comprise an amino acid sequence having at least about 70%, at least about 75%, at least about 80%, at least about 85%, at least about 90%, at least about 95%, at least about 98%, at least about 99%, or 100%, amino acid sequence identity to amino acids 384-751 of an amino acid sequence identified as 2A and depicted in FIGS. 9A-C. For example, an E2 polypeptide of genotype 2B can comprise an amino acid sequence having at least about 70%, at least about 75%, at least about 80%, at least about 85%, at least about 90%, at least about 95%, at least about 98%, at least about 99%, or 100%, amino acid sequence identity to amino acids 384-751 of an amino acid sequence identified as 2B and depicted in FIGS. 9A-C.

### Modified E2 Polypeptide

In some cases, the E2 polypeptide present in a subject immunogenic composition is modified such that the E2 polypeptide has reduced glycosylation compared to wild-type E2 polypeptide. Wild-type HCV E2 is glycosylated at E2N1 and/or E2N6, which in the reference strain H77 includes residues Asn-417 and/or Asn-532, respectively. “E2N1” and “E2N6” refer to amino acid sequence motifs at which naturally-occurring HCV E2 polypeptides can be N-linked glycosylated, which motifs are positioned in the E2 polypeptide as shown in the alignments of FIGS. 1A-1C, 2A-2E and 5. For example, as shown in FIGS. 1A-C, in the reference genotype 1 strain H77, the E2N1 site includes Asn-417 and the E2N6 site includes Asn-532. As another example, as shown in FIGS. 2A and 2B, the E2N1 site includes Asn-418, and the E2N6 site includes Asn-534. As another example, as shown in FIGS. 8A and 8B, the E2N1 site includes Asn-417, and the E2N6 site includes Asn-533.

A modified E2 polypeptide suitable for inclusion in a subject immunogenic composition has a substitution of the asparagine at E2N1 and/or E2N6 (e.g., by modification (e.g., substitution) of Asn-417 and/or Asn-532), such that N-linked glycosylation does not occur at these positions. E2 polypeptides for production of modified E2 polypeptides can be an E2 polypeptide of any genotype, subtype, or isolate of HCV. E2 polypeptides of genotype 1 and E2 polypeptides of genotype 3 are of particular interest.

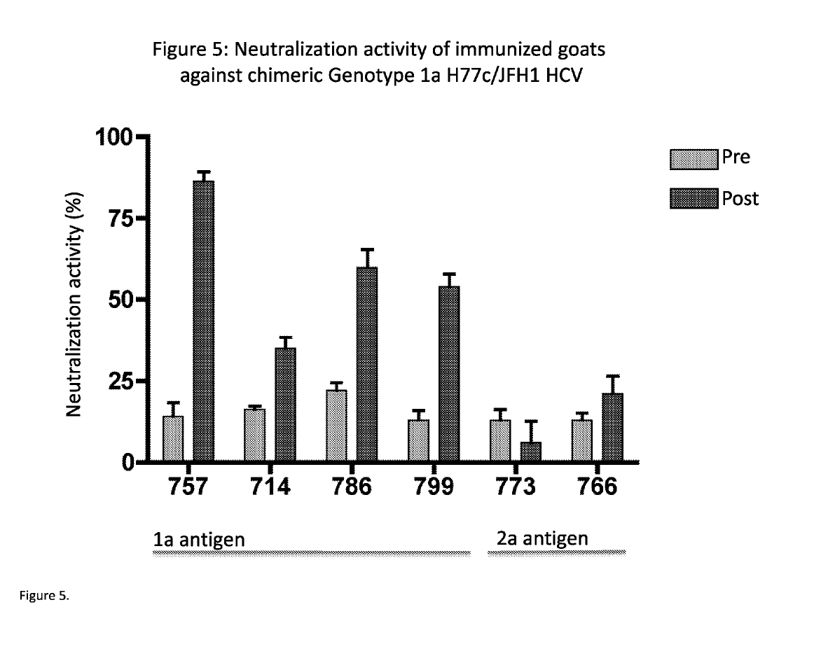

A modified E2 polypeptide can comprise a modification, e.g., a substitution, of an asparagine in the sites E2N1 and/or E2N6 (e.g., Asn-417 and/or Asn-532), such that N-linked glycosylation does not occur at these positions. The asparagine of E2N1 and/or E2N6 (e.g., Asn-417 and/or the Asn-532) can be substituted with any amino acid that is not subject to glycosylation (including N-glycosylation and O-glycosylation). Examples of substitutions include, but are not limited to, N417T (substitution of Asn-417 with a threonine), N417S (substitution of Asn-417 with a serine), N417Q (substitution of Asn-417 with a glutamine), N417Y (substitution of Asn-417 with a tyrosine), N417C (substitution of Asn-417 with a cysteine), N532T, N532S, N532Q, N532Y, N532C, and the like. The position of the substituted asparagine(s) is based on the numbering depicted in FIGS. 1A-C(HCV genotype 1). The positions of the substituted asparagine(s) in genotype 3 are shown by arrows in FIGS. 2A-2B and FIG. 3. Positions of substituted asparagine(s) are also depicted in FIG. 5.

A modified E2 polypeptide can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa.

In FIGS. 1A-C, the amino acid sequence of E2 is amino acid 384 to amino acid 746. In FIGS. 2A-2B, the amino acid sequence of E2 is amino acid 385 to amino acid 754. In FIG. 3, the amino acid sequence of E2 is amino acid 386 to amino acid 746. As used herein, an “E2 polypeptide” includes a precursor E2 protein, including the signal sequence; includes a mature E2 polypeptide which lacks this sequence; and includes an E2 polypeptide with a heterologous signal sequence. An E2 polypeptide can include a C-terminal membrane anchor sequence which occurs at approximately amino acid positions 715-730 and may extend as far as approximately amino acid residue 746 (see, Lin et al., J. Virol. (1994) 68:5063-5073).

In some cases, a modified E2 polypeptide lacks a portion of its C-terminal region, e.g., from about amino acid 715 to the C-terminus; from about amino acid 625 to the C-terminus; from about amino acid 650 to the C-terminus; from about amino acid 661 to the C-terminus; from about amino acid 655 to the C-terminus; from about amino acid 500 to the C-terminus, where the amino acid numbering is with reference to the numbering in FIGS. 1A-C. See, e.g., U.S. Pat. No. 6,521,423.

A modified E2 polypeptide can comprise an amino acid sequence having at least about 70%, at least about 75%, at least about 80%, at least about 85%, at least about 90%, at least about 95%, at least about 98%, at least about 99%, or 100%, amino acid sequence identity to the amino acid sequence of an E2 polypeptide depicted in FIGS. 1A-C, FIGS. 2A-2B, or FIG. 3, where the modified E2 polypeptide has an amino acid substitution of asparagine at E2N1 and/or E2N6 (e.g., Asn-417 and/or Asn-532).

A modified E2 polypeptide can comprise an amino acid sequence having at least about 70%, at least about 75%, at least about 80%, at least about 85%, at least about 90%, at least about 95%, at least about 98%, at least about 99%, or 100%, amino acid sequence identity to the amino acid sequence of an E2 polypeptide depicted in FIGS. 1A-C, FIGS. 2A-F, or FIG. 3, where the modified E2 polypeptide has an amino acid substitution of asparagine at E2N2 (e.g., Asn-417), where the amino acid sequence comprises an asparagine at E2N6 (e.g., Asn-532).

A modified E2 polypeptide can comprise an amino acid sequence having at least about 70%, at least about 75%, at least about 80%, at least about 85%, at least about 90%, at least about 95%, at least about 98%, at least about 99%, or 100%, amino acid sequence identity to the amino acid sequence of an E2 polypeptide depicted in FIGS. 1A-C, FIGS. 2A-F, or FIG. 3, where the modified E2 polypeptide has an amino acid substitution of asparagine at E2N6 (e.g., Asn-532), where the amino acid sequence comprises asparagine at E2N1 (e.g., Asn-417).

A modified E2 polypeptide can comprise an amino acid sequence having at least about 70%, at least about 75%, at least about 80%, at least about 85%, at least about 90%, at least about 95%, at least about 98%, at least about 99%, or 100%, amino acid sequence identity to the amino acid sequence of an E2 polypeptide depicted in FIGS. 1A-C, FIGS. 2A-F, or FIG. 3, where the modified E2 polypeptide has an amino acid substitution of asparagine at E2N1 (e.g., Asn-417) and an amino acid substitution of asparagine at E2N6 (e.g., Asn-532).

In some cases, in addition to the above-described modifications, a modified E2 polypeptide includes an amino acid substitution of an asparagine at one or more of: 1) the E2N2 site (e.g., residue 423); 2) the E2N4 site (e.g., residue 448); 3) the E2N11 site (e.g., residue 645).

### Compositions Comprising HCV E1 of HCV Genotype 1

The present disclosure provides an immunogenic composition comprising E1/E2 of HCV genotype 1. In some cases, the immunogenic composition comprises HCV genotype 1 E1/E2 alone, e.g., the immunogenic composition does not include an E1 polypeptide, an E2 polypeptide, or an E1/E2 heterodimer of any other HCV genotype.

Compositions Comprising E1, E2, and/or E1/E2 of Two Different HCV Genotypes

Compositions of the present disclosure comprise: a) an HCV E1 polypeptide, an E2 polypeptide, or an E1/E2 heterodimeric polypeptide complex from a first HCV genotype; b) an HCV E1 polypeptide, an E2 polypeptide, or an E1/E2 heterodimeric polypeptide complex from a second HCV genotype; and c) a pharmaceutically acceptable excipient, with the proviso that the composition comprises at least one E1 polypeptide and at least one E2 polypeptide. The E2 polypeptide can be present in the composition in a heterodimeric complex with an E1 polypeptide, or can be present in the composition uncomplexed with an E1 polypeptide.

In the compositions described below, “E2” refers to an E2 polypeptide uncomplexed with an E1 polypeptide, while “E1/E2” refers to E1 and E2 present together in a heterodimeric polypeptide complex. Similarly, the E1 polypeptide can be present in the composition in a heterodimeric complex with an E2 polypeptide, or can be present in the composition uncomplexed with an E2 polypeptide. In the compositions described below, “E1” refers to an E1 polypeptide uncomplexed with an E2 polypeptide, while “E1/E2” refers to E1 and E2 present together in a heterodimeric polypeptide complex.

Composition of the present disclosure can comprise:

a) an E1/E2 heterodimeric polypeptide complex of a first HCV genotype; and an E1/E2 heterodimeric polypeptide complex of a second HCV genotype;

b) an E1/E2 heterodimeric polypeptide complex of a first HCV genotype; and an E2 polypeptide of a second HCV genotype;

c) an E2 polypeptide of a first HCV genotype; and an E1/E2 heterodimeric polypeptide complex of a second HCV genotype;

d) an E1 polypeptide of a first HCV genotype; and an E2 polypeptide of a second HCV genotype;

e) an E1 polypeptide of a first HCV genotype; and an E1/E2 heterodimeric polypeptide complex of a second HCV genotype;

f) an E2 polypeptide of a first HCV genotype; and an E1 polypeptide of a second HCV genotype; or

g) an E2 polypeptide of a first HCV genotype; and an E1/E2 heterodimeric polypeptide complex of a second HCV genotype.

Exemplary compositions of the present disclosure include:

1) an E1/E2 heterodimeric polypeptide complex of HCV genotype 1; and an E1/E2 heterodimeric polypeptide complex of HCV genotype 3, e.g., where all of the polypeptides are wild-type;

2) an E1/E2 heterodimeric polypeptide complex of HCV genotype 1; and an E2 polypeptide of HCV genotype 3, e.g., where all of the polypeptides are wild-type;

3) an E2 polypeptide of HCV genotype 1; and an E1/E2 heterodimeric polypeptide complex of HCV genotype 3, e.g., where all of the polypeptides are wild-type;

4) an E1/E2 heterodimeric polypeptide complex of HCV genotype 1; and an E1/E2 heterodimeric polypeptide complex of HCV genotype 2, e.g., where all of the polypeptides are wild-type;

5) an E1/E2 heterodimeric polypeptide complex of HCV genotype 1; and an E2 polypeptide of HCV genotype 2, e.g., where all of the polypeptides are wild-type;

6) an E2 polypeptide of HCV genotype 1; and an E1/E2 heterodimeric polypeptide complex of HCV genotype 2, e.g., where all of the polypeptides are wild-type;

7) an E1 polypeptide of HCV genotype 1; and an E2 polypeptide of HCV genotype 3, e.g., where all of the polypeptides are wild-type;

8) an E1 polypeptide of HCV genotype 1; and an E1/E2 heterodimeric polypeptide complex of HCV genotype 3, e.g., where all of the polypeptides are wild-type;

9) an E2 polypeptide of HCV genotype 1; and an E1 polypeptide of HCV genotype 3, e.g., where all of the polypeptides are wild-type;

10) an E2 polypeptide of HCV genotype 1; and an E1/E2 heterodimeric polypeptide complex of HCV genotype 3, e.g., where all of the polypeptides are wild-type;

11) an E1 polypeptide of HCV genotype 1; and an E2 polypeptide of HCV genotype 2, e.g., where all of the polypeptides are wild-type;

12) an E1 polypeptide of HCV genotype 1; and an E1/E2 heterodimeric polypeptide complex of HCV genotype 2, e.g., where all of the polypeptides are wild-type;

13) an E2 polypeptide of HCV genotype 1; and an E1 polypeptide of HCV genotype 2, e.g., where all of the polypeptides are wild-type;

14) an E2 polypeptide of HCV genotype 1; and an E1/E2 heterodimeric polypeptide complex of HCV genotype 2, e.g., where all of the polypeptides are wild-type.

In some embodiments, a subject composition does not include an E1 polypeptide, and E2 polypeptide, and/or an E1/E2 polypeptide of any HCV genotype other than the above-mentioned genotype 1 and 2 combinations or genotype 1 and 3 combinations.

As noted above, the E2 polypeptide can have a wild-type HCV E2 amino acid sequence, e.g., where the E2 polypeptide does not have an amino acid substitution of asparagine in an E2N1 site and/or an E2N6 site. A “wild-type” HCV E2 polypeptide comprises an amino acid sequence of an HCV E2 polypeptide found in nature.

As noted above, in some embodiments, the E2 polypeptide can comprise an amino acid substitution of asparagine in an E2N1 site and/or an E2N6 site. The following are exemplary compositions:

15) an E1/E2 heterodimeric polypeptide complex of HCV genotype 1, where the E2 polypeptide comprises a modification (e.g., a substitution) of an asparagine at the E2N1 and/or E2N6 site such as described above; and an E1/E2 heterodimeric polypeptide complex of HCV genotype 3, where the E2 polypeptide is wild-type;

16) an E1/E2 heterodimeric polypeptide complex of HCV genotype 1, where the E2 polypeptide is wild-type; and an E1/E2 heterodimeric polypeptide complex of HCV genotype 3, where the E2 polypeptide comprises a modification (e.g., a substitution) of an asparagine at the E2N1 and/or E2N6 site such as described above;

17) an E1/E2 heterodimeric polypeptide complex of HCV genotype 1, where the E2 polypeptide comprises a modification (e.g., a substitution) of an asparagine at the E2N1 and/or E2N6 site such as described above; and an E2 polypeptide of HCV genotype 3, where the E2 polypeptide is wild-type;

18) an E1/E2 heterodimeric polypeptide complex of HCV genotype 1, where the E2 polypeptide is wild-type; and an E2 polypeptide of HCV genotype 3, where the E2 polypeptide comprises a modification (e.g., a substitution) of an asparagine at the E2N1 and/or E2N6 site such as described above;

19) an E2 polypeptide of HCV genotype 1, where the E2 polypeptide comprises a modification (e.g., a substitution) of an asparagine at the E2N1 and/or E2N6 site such as described above; and an E1/E2 heterodimeric polypeptide complex of HCV genotype 3, where the E2 polypeptide is wild-type;

20) an E2 polypeptide of HCV genotype 1, where the E2 polypeptide is wild-type; and an E1/E2 heterodimeric polypeptide complex of HCV genotype 3, where the E2 polypeptide comprises a modification (e.g., a substitution) of an asparagine at the E2N1 and/or E2N6 site such as described above;

21) an E1/E2 heterodimeric polypeptide complex of HCV genotype 1, where the E2 polypeptide comprises a modification (e.g., a substitution) of an asparagine at the E2N1 and/or E2N6 site such as described above; and an E1/E2 heterodimeric polypeptide complex of HCV genotype 2, where the E2 polypeptide is wild-type;

22) an E1/E2 heterodimeric polypeptide complex of HCV genotype 1, where the E2 polypeptide is wild-type; and an E1/E2 heterodimeric polypeptide complex of HCV genotype 2, where the E2 polypeptide comprises a modification (e.g., a substitution) of an asparagine at the E2N1 and/or E2N6 site such as described above;

23) an E1/E2 heterodimeric polypeptide complex of HCV genotype 1, where the E2 polypeptide comprises a modification (e.g., a substitution) of an asparagine at the E2N1 and/or E2N6 site such as described above; and an E2 polypeptide of HCV genotype 2, where the E2 polypeptide is wild-type;

24) an E1/E2 heterodimeric polypeptide complex of HCV genotype 1, where the E2 polypeptide is wild-type; and an E2 polypeptide of HCV genotype 2, where the E2 polypeptide comprises a modification (e.g., a substitution) of an asparagine at the E2N1 and/or E2N6 site such as described above;

25) an E2 polypeptide of HCV genotype 1, where the E2 polypeptide comprises a modification (e.g., a substitution) of an asparagine at the E2N1 and/or E2N6 site such as described above; and an E1/E2 heterodimeric polypeptide complex of HCV genotype 2, where the E2 polypeptide is wild-type;

26) an E2 polypeptide of HCV genotype 1, where the E2 polypeptide is wild-type; and an E1/E2 heterodimeric polypeptide complex of HCV genotype 2, where the E2 polypeptide comprises a modification (e.g., a substitution) of an asparagine at the E2N1 and/or E2N6 site such as described above.

### Compositions Comprising E2 or E1/E2 of Two Different HCV Genotypes

Compositions of the present disclosure comprise: a) an HCV E2 polypeptide, or an E1/E2 heterodimeric polypeptide complex from a first HCV genotype; b) an HCV E2 polypeptide, or an E1/E2 heterodimeric polypeptide complex, from a second HCV genotype; and c) a pharmaceutically acceptable excipient. The E2 polypeptide can be present in the composition in a heterodimeric complex with an E1 polypeptide, or can be present in the composition uncomplexed with an E1 polypeptide. The E2 polypeptide can be soluble.

A composition of the present disclosure can comprise one of a) through k), as described below, where the composition comprises a pharmaceutically acceptable excipient:

a) an E2 polypeptide of HCV genotype 1a; and an E1/E2 heterodimeric polypeptide complex of HCV genotype 3a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

b) a full-length E2 polypeptide of HCV genotype 1a; and an E1/E2 heterodimeric polypeptide complex of HCV genotype 3a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

c) a soluble E2 polypeptide of HCV genotype 1a; and an E1/E2 heterodimeric polypeptide complex of HCV genotype 3a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

d) an E2 polypeptide of HCV genotype 1a, where the E2 polypeptide comprises a modification (e.g., a substitution) of an asparagine at the E2N1 and/or E2N6 site such as described above; and an E1/E2 heterodimeric polypeptide complex of HCV genotype 3a, where the E2 polypeptide of the E1/E2 heterodimer is wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

e) an E2 polypeptide of HCV genotype 1a, where the E2 polypeptide is wild-type; and an E1/E2 heterodimeric polypeptide complex of HCV genotype 3a, where the E2 polypeptide of the E1/E2 heterodimer comprises a modification (e.g., a substitution) of an asparagine at the E2N1 and/or E2N6 site such as described above. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

f) an E2 polypeptide of HCV genotype 3a; and an E1/E2 heterodimeric polypeptide complex of HCV genotype 1a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

g) a full-length E2 polypeptide of HCV genotype 3a; and an E1/E2 heterodimeric polypeptide complex of HCV genotype 1a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

h) a soluble E2 polypeptide of HCV genotype 3a; and an E1/E2 heterodimeric polypeptide complex of HCV genotype 1a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

i) an E2 polypeptide of HCV genotype 3a, where the E2 polypeptide comprises a modification (e.g., a substitution) of an asparagine at the E2N1 and/or E2N6 site such as described above; and an E1/E2 heterodimeric polypeptide complex of HCV genotype 1a, where the E2 polypeptide of the E1/E2 heterodimer is wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

j) an E2 polypeptide of HCV genotype 3a, where the E2 polypeptide is wild-type; and an E1/E2 heterodimeric polypeptide complex of HCV genotype 1a, where the E2 polypeptide of the E1/E2 heterodimer comprises a modification (e.g., a substitution) of an asparagine at the E2N1 and/or E2N6 site such as described above. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

k) an E2 polypeptide or an E1/E2 heterodimeric polypeptide complex of HCV genotype 1a; and an E2 polypeptide or an E1/E2 heterodimeric polypeptide complex of HCV genotype 2a. In some cases, all of the polypeptides are wild-type. In some cases, the genotype 1a and the genotype 2a polypeptides are both E1/E2 heterodimeric polypeptide. In some cases, the genotype 1a polypeptide is an E2 polypeptide; and the genotype 2a polypeptide is an E1/E2 heterodimeric polypeptide complex. In some cases, the genotype 1a polypeptide is an E1/E2 heterodimeric polypeptide complex; and the genotype 2a polypeptide is an E2 polypeptide. In some cases, the genotype 1a and the genotype 2a polypeptides are both E2 polypeptides. In some cases, the genotype 1a and the genotype 2a polypeptides are both soluble E2 polypeptides. In some cases, the composition is administered in an amount that is effective to neutralize HCV genotypes such as genotypes 1a, 1b, 2a, 4, 5, and 6a. In some cases, the composition is administered in an amount that is effective to neutralize HCV genotypes 1a and 2a. In some cases, the composition is administered in an amount that is effective to induce neutralizing antibody to HCV genotypes 1a and 2a. In some cases, the composition is administered in an amount that is effective to induce a CTL response to HCV genotypes 1a and 2a. In some cases, the composition is administered in an amount that is effective to achieve 50% or greater than 50% neutralization of HCV genotypes 1a and 2a. In some cases, the composition is administered in an amount that is effective to achieve 50% or greater than 50% neutralization of HCV genotypes 1a, 1b, 2a, 4, 5, and 6a. In some embodiments, the composition does not include an E1 polypeptide, an E2 polypeptide, and/or an E1/E2 polypeptide of any HCV genotype other than the above-mentioned genotypes 1a and 2a. In some embodiments, a subject composition also includes an E1 polypeptide, and E2 polypeptide, and/or an E1/E2 polypeptide of at least one additional HCV genotype.

In any of the above-described embodiments, E2 (either as uncomplexed E2 or as an E1/E2 heterodimeric complex) of HCV genotype 1a can be substituted with E2 (either as uncomplexed E2 or as an E1/E2 heterodimeric complex) of HCV genotype 1, 4, 5, or 6.

Compositions Comprising E1, E2, and/or E1/E2 of Three Different HCV Genotypes

Compositions of the present disclosure comprise: a) an HCV E1 polypeptide, an E2 polypeptide, or an E1/E2 heterodimeric polypeptide complex from a first HCV genotype; b) an HCV E1 polypeptide, an E2 polypeptide, or an E1/E2 heterodimeric polypeptide complex from a second HCV genotype; c) an HCV E1 polypeptide, an E2 polypeptide, or an E1/E2 heterodimeric polypeptide complex from a third HCV genotype; and d) a pharmaceutically acceptable excipient, with the proviso that the composition comprises at least one E1 polypeptide and at least one E2 polypeptide. The E2 polypeptide can be present in the composition in a heterodimeric complex with an E1 polypeptide, or can be present in the composition uncomplexed with an E1 polypeptide. In the compositions described below, “E2” refers to an E2 polypeptide uncomplexed with an E1 polypeptide, while “E1/E2” refers to E1 and E2 present together in a heterodimeric polypeptide complex. Similarly, the E1 polypeptide can be present in the composition in a heterodimeric complex with an E2 polypeptide, or can be present in the composition uncomplexed with an E2 polypeptide. In the compositions described below, “E1” refers to an E1 polypeptide uncomplexed with an E2 polypeptide, while “E1/E2” refers to E1 and E2 present together in a heterodimeric polypeptide complex.

Composition of the Present Disclosure can Comprise:

i) an E1/E2 heterodimeric polypeptide complex of HCV genotype 1; an E1/E2 heterodimeric polypeptide complex of HCV genotype 2; and an E1/E2 heterodimeric polypeptide complex of HCV genotype 3, e.g., where all of the polypeptides are wild-type;

ii) an E1/E2 heterodimeric polypeptide complex of HCV genotype 1; an E1/E2 heterodimeric polypeptide complex of HCV genotype 2; and an E2 polypeptide of HCV genotype 3, e.g., where all of the polypeptides are wild-type;

iii) an E1/E2 heterodimeric polypeptide complex of HCV genotype 1; an E2 polypeptide of HCV genotype 2; and an E1/E2 heterodimeric polypeptide complex of HCV genotype 3, e.g., where all of the polypeptides are wild-type;

iv) an E2 polypeptide of HCV genotype 1; an E1/E2 heterodimeric polypeptide complex of HCV genotype 2; and an E1/E2 heterodimeric polypeptide complex of HCV genotype 3, e.g., where all of the polypeptides are wild-type;

v) an E1/E2 heterodimeric polypeptide complex of HCV genotype 1; an E2 polypeptide of HCV genotype 2; and an E2 polypeptide of HCV genotype 3, e.g., where all of the polypeptides are wild-type;

vi) an E2 polypeptide of HCV genotype 1; an E1/E2 heterodimeric polypeptide complex of HCV genotype 2; and an E2 polypeptide of HCV genotype 3, e.g., where all of the polypeptides are wild-type;

vii) an E2 polypeptide of HCV genotype 1; an E2 polypeptide of HCV genotype 2; and an E1/E2 heterodimeric polypeptide complex of HCV genotype 3, e.g., where all of the polypeptides are wild-type.

viii) an E1 polypeptide of HCV genotype 1; an E1/E2 heterodimeric polypeptide complex of HCV genotype 2; and an E1/E2 heterodimeric polypeptide complex of HCV genotype 3;

ix) an E1/E2 heterodimeric polypeptide complex of HCV genotype 1; an E1 polypeptide of HCV genotype 2; and an E1/E2 heterodimeric polypeptide complex of HCV genotype 3;

x) an E1/E2 heterodimeric polypeptide complex of HCV genotype 1; an E1/E2 heterodimeric polypeptide complex of HCV genotype 2; and an E1 polypeptide of HCV genotype 3;

xi) an E1 polypeptide of HCV genotype 1; an E2 polypeptide of HCV genotype 2; and an E1/E2 heterodimeric polypeptide complex of HCV genotype 3;

xii) an E2 polypeptide of HCV genotype 1; an E1 polypeptide of HCV genotype 2; and an E1/E2 heterodimeric polypeptide complex of HCV genotype 3;

xiii) an E1/E2 heterodimeric polypeptide complex of HCV genotype 1; an E2 polypeptide of HCV genotype 2; and an E1 polypeptide of HCV genotype 3;

xiv) an E1 polypeptide of HCV genotype 1; an E2 polypeptide of HCV genotype 2; and an E2 polypeptide of HCV genotype 3;

xv) an E2 polypeptide of HCV genotype 1; an E1 polypeptide of HCV genotype 2; and an E2 polypeptide of HCV genotype 3;

xvi) an E2 polypeptide of HCV genotype 1; an E2 polypeptide of HCV genotype 2; and an E1 polypeptide of HCV genotype 3;

xvii) an E1 polypeptide of HCV genotype 1; an E1/E2 heterodimeric polypeptide complex of HCV genotype 2; and an E2 polypeptide of HCV genotype 3.

In some embodiments, a subject composition does not include an E1 polypeptide, and E2 polypeptide, and/or an E1/E2 polypeptide of any HCV genotype other than the above-mentioned genotypes 1, 2, and 3. In some embodiments, a subject composition also includes an E1 polypeptide, and E2 polypeptide, and/or an E1/E2 polypeptide of HCV genotype 7.

In any of the above-noted compositions, the E2 polypeptide can have a wild-type HCV E2 amino acid sequence, e.g., where the E2 polypeptide does not have an amino acid substitution of asparagine in an E2N1 site and/or an E2N6 site. A “wild-type” HCV E2 polypeptide comprises an amino acid sequence of an HCV E2 polypeptide found in nature. In some embodiments, an E2 polypeptide can comprise an amino acid substitution of asparagine in an E2N1 site and/or an E2N6 site.

Compositions Comprising E2, and/or E1/E2 of Three Different HCV Genotypes

Compositions of the present disclosure comprise: a) an HCV E2 polypeptide, or an E1/E2 heterodimeric polypeptide complex from a first HCV genotype; b) an HCV E2 polypeptide, or an E1/E2 heterodimeric polypeptide complex from a second HCV genotype; c) an HCV E2 polypeptide, or an E1/E2 heterodimeric polypeptide complex from a third HCV genotype; and d) a pharmaceutically acceptable excipient. The E2 polypeptide can be present in the composition in a heterodimeric complex with an E1 polypeptide, or can be present in the composition uncomplexed with an E1 polypeptide. The E2 polypeptide can be soluble.

A composition of the present disclosure can comprise one of a) through f), as described below, where the composition comprises a pharmaceutically acceptable excipient:

a) an E2 polypeptide of HCV genotype 1a; an E1/E2 heterodimeric polypeptide complex of HCV genotype 2a; and an E2 polypeptide of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

b) an E2 polypeptide of HCV genotype 1a; an E2 polypeptide of HCV genotype 2a; and an E1/E2 heterodimeric polypeptide complex of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

c) an E1/E2 heterodimeric polypeptide complex of HCV genotype 1a; an E2 polypeptide of HCV genotype 2a; and an E2 polypeptide of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

d) an E1/E2 heterodimeric polypeptide complex of HCV genotype 1a; an E1/E2 heterodimeric polypeptide complex of HCV genotype 2a; and an E2 polypeptide of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

e) an E2 polypeptide of HCV genotype 1a; an E1/E2 heterodimeric polypeptide complex of HCV genotype 2a; and an E1/E2 heterodimeric polypeptide complex of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

f) an E1/E2 heterodimeric polypeptide complex of HCV genotype 1a; an E2 polypeptide of HCV genotype 2a; and an E1/E2 heterodimeric polypeptide complex of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

### Methods of Making E1 and E2 Polypeptides

E1 and E2 polypeptides can be produced using any suitable method, including recombinant and non-recombinant methods (e.g., chemical synthesis).

Where a polypeptide is chemically synthesized, the synthesis may proceed via liquid phase or solid-phase. Solid-phase peptide synthesis (SPPS) allows the incorporation of unnatural amino acids and/or peptide/protein backbone modification. Various forms of SPPS, such as Fmoc and Boc, are available for synthesizing polypeptides. Details of the chemical synthesis are known in the art (e.g., Ganesan A. 2006 Mini Rev. Med Chem. 6:3-10 and Camarero J A et al. 2005 Protein Pept Lett. 12:723-8).

Where a polypeptide is produced using recombinant techniques, the polypeptide may be produced as an intracellular protein or as an secreted protein, using any suitable construct and any suitable host cell, which can be a prokaryotic or eukaryotic cell, such as a bacterial (e.g., Escherichia coli) cell or a yeast host cell, respectively. Other examples of eukaryotic cells that may be used as host cells include insect cells, mammalian cells, filamentous fungi, and plant cells. Suitable yeast cells include, e.g., Saccharomyces cerevisiae and Pichia (e.g., Pichia pastoris).

Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, MRCS cells (ATCC No. CCL-171), and the like. Where mammalian host cells are used, such host cells may include human cells (e.g., HeLa, 293, H9 and Jurkat cells); mouse cells (e.g., NIH3T3, L cells, and C127 cells); primate cells (e.g., Cos 1, Cos 7 and CV1) and hamster cells (e.g., Chinese hamster ovary (CHO) cells).

A variety of host-vector systems suitable for the expression of a polypeptide may be employed according to standard procedures known in the art. See, e.g., Sambrook et al., 1989 Current Protocols in Molecular Biology Cold Spring Harbor Press, New York; Ausubel et al. 1995 Current Protocols in Molecular Biology, Eds. Wiley and Sons; “Protein Expression: A Practical Approach” (1999) S. J. Higgins and B. D. James, eds., Oxford University Press; “Protein Expression in Mammalian Cells: Methods and Protocols (Methods in Molecular Biology)” (2012) James L. Hartley, ed., Humana Press; and “Production of Recombinant Proteins” (2005) Gerd Gellisen, ed., Wiley-VCH. Methods for introduction of nucleic acids into host cells include, for example, transformation, electroporation, conjugation, transfection, calcium phosphate methods, and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically-integrated. A variety of appropriate vectors for use in production of a peptide of interest are available commercially.

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, HIV-based lentivirus vectors, murine leukemia virus (MVL)-based gamma retrovirus vectors, and the like), P1-based artificial chromosomes, yeast plasmids, yeast artificial chromosomes, and any other vectors specific for specific hosts of interest (such as E. coli, mammalian cells, insect cells, or yeast cells).

E1 and E2 polypeptides can be produced by introducing a recombinant expression vector comprising a nucleotide sequence encoding the E1 and/or the E2 polypeptide into an appropriate host cell, where the host cell produces the encoded E1 and/or E1 polypeptide. In the expression vector, a polynucleotide comprising a nucleotide sequence(s) encoding E1 and/or E2 is linked to a regulatory sequence as appropriate to obtain the desired expression properties. These regulatory sequences can include promoters, enhancers, terminators, operators, repressors, and inducers. The promoters can be regulated or constitutive. Expression vectors generally have convenient restriction sites located near the promoter sequence to provide for the insertion of nucleic acid sequences encoding a protein of interest. A selectable marker operative in the expression host cell may be present.

In some cases, the E1 and E2 polypeptides are encoded in a recombinant expression vector suitable for expression in a eukaryotic host cell (e.g., an insect cell; a yeast cell; a mammalian host cell, such as CHO cells, HeLa cells, 293 cells, MRCS cells, etc.). In some cases, a recombinant expression vector comprises a nucleotide sequence encoding E1 and E2 as a single polypeptide chain; the recombinant expression vector is introduced into a eukaryotic host cell to generate a genetically modified host cell. The E1 and E2 polypeptides are initially produced as a single polypeptide chain, which is cleaved in the endoplasmic reticulum (ER) of the genetically modified host cell to produce separate E1 and E2 polypeptides. The separate E1 and E2 polypeptides can form a heterodimer (e.g., a non-covalently linked heterodimer) in the ER. The E1/E2 heterodimer can be isolated from the genetically modified host cell by, e.g., lysis using a non-ionic detergent. See, e.g., Frey et al. (2010) Vaccine 28:6367.

Alternatively, the E1 and E2 polypeptides can be encoded on separate recombinant expression vectors; and produced in a cell (e.g., the same host cell or separate host cells) as separate polypeptides.

If full-length E1 and E2 polypeptides are expressed in a eukaryotic host cell, the E1 and E2 polypeptides remain in the lumen of the endoplasmic reticulum (ER) as asialoglycoproteins. If the E1 and E2 polypeptides have C-terminal truncations, such that the C-terminal transmembrane regions are removed, the truncated polypeptides are secreted as complex glycoproteins (where the glycosylations include sialic acid). An E1 polypeptide that has a C-terminal truncation, such that the C-terminal transmembrane removed, and such that the E1 polypeptide is secreted from a eukaryotic cell producing the E1 polypeptide, is referred to as a “soluble E1 polypeptide.” Similarly, an E2 polypeptide that has a C-terminal truncation, such that the C-terminal transmembrane removed, and such that the E2 polypeptide is secreted from a eukaryotic cell producing the E2 polypeptide, is referred to as a “soluble E2 polypeptide.” Removal of approximately amino acids 662-746 of E2, or amino acids 715-746 of D2, and removal of approximately amino acids 330-384 of E1, results in secretion of E2 and E1 from a eukaryotic host cell. If E1 and E2 are co-expressed in the same eukaryotic host cell as full-length polypeptides, they remain in the lumen of the ER as a heterodimer.

After production in a host cell, the E1 and E2 polypeptides (e.g., separately or as a heterodimer) can be purified from the host cell. Methods of purification of recombinantly produced polypeptides from a host cell are known in the art and include, e.g., detergent lysis (e.g., with a non-ionic detergent), followed by one or more of size exclusion column chromatography, high performance liquid chromatography, affinity chromatography, and the like.

### Formulations

HCV E1, E2, and E1/E2 polypeptides can be formulated with a pharmaceutically acceptable excipient(s) to generate a subject immunogenic composition. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc.

In some embodiments, the E1 and E2 polypeptides are formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 (TWEEN®20) or polysorbate 80 (TWEEN®80). For example, a formulation of E1 and E2 polypeptides in an aqueous buffer can include, e.g., from about 0.01% to about 0.05% polysorbate-20 (TWEEN®20) non-ionic detergent. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

E1 and E2 polypeptides can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An immunogenic composition of the present disclosure can include, e.g., pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The concentration of HCV E1, E2, and E1/E2 polypeptides in a formulation can vary widely (e.g., from less than about 0.1% to at least about 2%, to as much as 20% to 50% or more by weight) and can be selected primarily based on fluid volumes, viscosities, and patient-based factors in accordance with the particular mode of administration selected and the patient's needs.

The HCV polypeptide-containing formulations of the present disclosure can be provided in the form of a solution, suspension, tablet, pill, capsule, powder, gel, cream, lotion, ointment, aerosol or the like. It is recognized that oral administration can require protection of the compositions from digestion. This is typically accomplished either by association of the composition with an agent that renders it resistant to acidic and enzymatic hydrolysis or by packaging the composition in an appropriately resistant carrier. Means of protecting from digestion are well known in the art.

The HCV polypeptide-containing formulations of the present disclosure can also be provided so as to enhance serum half-life of the heterodimer following administration. For example, where isolated HCV E1, E2, or E1/E2 polypeptides are formulated for injection, the HCV polypeptide may be provided in a liposome formulation, prepared as a colloid, or other conventional techniques for extending serum half-life. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028. The preparations may also be provided in controlled release or slow-release forms.

### Adjuvant

An immunogenic composition of the present disclosure can include an adjuvant. Examples of known suitable adjuvants that can be used in humans include, but are not necessarily limited to, alum, aluminum phosphate, aluminum hydroxide, MF59 (4.3% w/v squalene, 0.5% w/v Tween 80™, 0.5% w/v Span 85), CpG-containing nucleic acid (where the cytosine is unmethylated), QS21, MPL, 3DMPL, extracts from Aquilla, ISCOMS, LT/CT mutants, poly(D,L-lactide-co-glycolide) (PLG) microparticles, Quil A, interleukins, and the like. For experimental animals, one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by one or more of measuring the amount of antibodies directed against the immunogenic antigen or antigenic epitope thereof, measuring a cytotoxic T lymphocyte response to the antigen, and measuring a helper T cell response to the antigen.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59TM (see, e.g., WO 90/14837), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), e.g., MPL+CWS (Detox™); (2) saponin adjuvants, such as QS21 or Stimulon™ (Cambridge Bioscience, Worcester, Mass.) may be used or particles generated therefrom such as ISCOMs (immunostimulating complexes), which ISCOMS may be devoid of additional detergent e.g. WO 00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO 00/56358; (6) combinations of 3dMPL with, for example, QS21 and/or oil-in-water emulsions (see, e.g. EP-A-0835318, EP-A-0735898, EP-A-0761231); (7) oligonucleotides comprising a CpG motif containing at least one CG dinucleotide, where the cytosine is unmethylated (see, e.g., WO 96/02555, WO 98/16247, WO 98/18810, WO 98/40100, WO 98/55495, WO 98/37919 and WO 98/52581); (8) a polyoxyethylene ether or a polyoxyethylene ester (see, e.g. WO 99/52549); (9) a polyoxyethylene sorbitan ester surfactant in combination with an octoxynol (WO 01/21207) or a polyoxyethylene alkyl ether or ester surfactant in combination with at least one additional non-ionic surfactant such as an octoxynol (WO 01/21152); (10) a saponin and an immunostimulatory oligonucleotide (e.g. a CpG oligonucleotide) (WO 00/62800); (11) an immunostimulant and a particle of metal salt (see, e.g. WO 00/23105); (12) a saponin and an oil-in-water emulsion (see e.g. WO 99/11241); (13) a saponin (e.g. QS21)+3dMPL+IM2 (optionally including a sterol) (see, e.g. WO 98/57659); (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc. Also suitable for use is Matrix-M™; Matrix-M™ is an adjuvant that comprises 40 nm nanoparticles comprising Quillaja saponins, cholesterol, and phospholipid. Adjuvants suitable for administration to a human are of particular interest. In some cases, the adjuvant is one that enhances a CD4+T helper response to the immunogen.

In some instances, the adjuvant is MF59, with or without a CpG-containing oligonucleotide. In other instances, the adjuvant is alum, with or without a CpG-containing oligonucleotide. In other instances, the adjuvant is poly(D,L-lactide-co-glycolide), with or without a CpG-containing oligonucleotide. In other instances, the adjuvant is MPL, with or without a CpG-containing oligonucleotide. In other cases, the adjuvant is Matrix-M™, with or without a CpG-containing oligonucleotide.

### Methods of Inducing an Immune Response to HCV

The present disclosure provides a method of inducing an immune response to at least one HCV genotype in a mammalian subject. The methods generally involve administering to an individual in need thereof an effective amount of a subject immunogenic composition.

An HCV immunogenic composition of the present disclosure is generally administered to a human subject who has an HCV infection or who is at risk of acquiring an HCV infection so as to prevent or at least partially arrest the development of disease and its complications. An amount adequate to accomplish this is defined as a “therapeutically effective dose” or a “therapeutically effective amount.” “Prophylactic” use of a subject immunogenic composition generally refers to administration to an individual who has not been infected with HCV. “Therapeutic” use of a subject immunogenic composition can refer to “prophylactic” use (administration to an individual who has not been infected with HCV) and/or to administration to an individual who has an HCV infection. A “therapeutically effective amount” of an immunogenic composition of the present disclosure, can be an amount that, when administered in one or more doses to an individual who is not infected with HCV, is effective to induce an immune response in the individual to HCV. A “therapeutically effective amount” of an immunogenic composition of the present disclosure, can be an amount that, when administered in one or more doses to an individual who is infected with HCV, is effective to enhance an immune response in the individual to HCV.

Amounts effective for therapeutic use will depend on, e.g., the immunogenic composition, the manner of administration, the weight and general state of health of the patient, and the judgment of the prescribing physician. Single or multiple doses of a subject immunogenic composition can be administered depending on the dosage and frequency required and tolerated by the patient, and route of administration.

In some cases, an effective amount (e.g., a therapeutically effective amount) of an HCV immunogenic composition of the present disclosure is an amount that, when administered to an individual in one or more doses, is effective to induce an antibody response to HCV in the individual. For example, antibody to HCV (e.g., extracellular HCV), and/or to an HCV-infected cell, can be induced.

An effective amount of an immunogenic composition of the present disclosure can be an amount that, when administered to an individual in one or more doses, is effective to induce a neutralizing antibody response to HCV of a variety of genotypes (e.g., genotype 1; genotype 3; genotype 2; genotype 7; genotype 5; genotype 6; etc.). A neutralizing antibody response reduces binding of HCV to CD81 and/or other cellular receptors, and inhibits entry of HCV into a cell. In some cases, an effective amount of an immunogenic composition of the present disclosure is an amount that is effective to induce a neutralizing antibody response to HCV of genotype 1 and genotype 3. In some cases, an effective amount of an immunogenic composition of the present disclosure is an amount that is effective to induce a neutralizing antibody response to HCV of genotype 1, genotype 2, and genotype 3. In some cases, an effective amount of an immunogenic composition of the present disclosure is an amount that is effective to induce a neutralizing antibody response to HCV of genotype 1, genotype 2, genotype 3, and genotype 7. In some cases, an effective amount of an immunogenic composition of the present disclosure is an amount that is effective to induce a neutralizing antibody response to HCV of genotype 1, genotype 2, genotype 3, genotype 4, genotype 5, genotype 6, and genotype 7. For example, in some cases, an effective amount of an immunogenic composition of the present disclosure is an amount that is effective to induce a neutralizing antibody response to HCV of genotype 1a/b and genotype 3a. As another example, in some cases, an effective amount of an immunogenic composition of the present disclosure is an amount that is effective to induce a neutralizing antibody response to HCV of genotype 1a/b, genotype 2a, and genotype 3a. As another example, in some cases, an effective amount of an immunogenic composition of the present disclosure is an amount that is effective to induce a neutralizing antibody response to HCV of genotype 1a/b, genotype 2b, and genotype 3a. As another example, in some cases, an effective amount of an immunogenic composition of the present disclosure is an amount that is effective to induce a neutralizing antibody response to HCV of genotype 1a/b, genotype 2a, genotype 3a, genotype 4a, genotype 5a, genotype 6a, and genotype 7a.

In some cases, an effective amount (e.g., a therapeutically effective amount) of an immunogenic composition of the present disclosure is an amount that, when administered to an individual in one or more doses, is effective to induce a cytotoxic T lymphocyte (CTL) response to HCV. For example, a CTL response to an HCV-infected cell can be induced.

In some cases, an effective amount of an immunogenic composition of the present disclosure is an amount that is effective to induce a CTL response to HCV of genotype 1 and genotype 3. In some cases, an effective amount of an immunogenic composition of the present disclosure is an amount that is effective to induce a CTL response to HCV of genotype 1, genotype 2, and genotype 3. In some cases, an effective amount of an immunogenic composition of the present disclosure is an amount that is effective to induce a CTL response to HCV of genotype 1, genotype 2, genotype 3, and genotype 7. In some cases, an effective amount of an immunogenic composition of the present disclosure is an amount that is effective to induce a CTL response to HCV of genotype 1, genotype 2, genotype 3, genotype 4, genotype 5, genotype 6, and genotype 7. For example, in some cases, an effective amount of an immunogenic composition of the present disclosure is an amount that is effective to induce a CTL response to HCV of genotype 1a/b and genotype 3a. As another example, in some cases, an effective amount of an immunogenic composition of the present disclosure is an amount that is effective to induce a CTL response to HCV of genotype 1a/b, genotype 2a, and genotype 3a. As another example, in some cases, an effective amount of an immunogenic composition of the present disclosure is an amount that is effective to induce a CTL response to HCV of genotype 1a/b, genotype 2b, and genotype 3a. As another example, in some cases, an effective amount of an immunogenic composition of the present disclosure is an amount that is effective to induce a CTL response to HCV of genotype 1a/b, genotype 2a, genotype 3a, genotype 4a, genotype 5a, genotype 6a, and genotype 7a.

In some cases, an effective amount (e.g., a therapeutically effective amount) of an immunogenic composition of the present disclosure is an amount that, when administered to an individual in one or more doses, is effective to induce a helper T lymphocyte (e.g., CD4+ T cell) to HCV in an individual.

In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV genotype 1. In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV genotype 3. In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV genotype 1 and HCV genotype 3. In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV of any genotype.

An immunogenic composition of the present disclosure is generally administered in an amount effective to elicit an immune response, e.g., a humoral immune response (e.g., an antibody response) and/or a CTL response, in the mammalian subject. Effective amounts for immunization will vary, and can generally range from about 1 μg to 100 μg per 70 kg patient, e.g., from about 5 μg/70 kg to about 50 μg/70 kg. Substantially higher dosages (e.g. 10 mg to 100 mg or more) may be suitable in oral, nasal, or topical administration routes. The initial administration can be followed by booster immunization of the same HCV immunogenic composition or a different HCV immunogenic composition. In some instances, a subject method of inducing an immune response involves an initial administration of an HCV immunogenic composition of the present disclosure, followed by at least one booster, and in some instances involves two or more (e.g., three, four, or five) boosters. The interval between an initial administration and a booster, or between a give booster and a subsequent booster, can be from about 1 week to about 12 weeks, e.g., from about 1 week to about 2 weeks, from about 2 weeks to about 4 weeks, from about 4 weeks to about 6 weeks, from about 6 weeks to about 8 weeks, from about 8 weeks to about 10 weeks, or from about 10 weeks to about 12 weeks.

In general, immunization can be accomplished by administration of an HCV immunogenic composition of the present disclosure by any suitable route, including administration of the composition orally, nasally, nasopharyngeally, parenterally, enterically, gastrically, topically, transdermally, subcutaneously, intramuscularly, in tablet, solid, powdered, liquid, aerosol form, locally or systemically, with or without added excipients. Actual methods for preparing parenterally administrable compositions will be known or apparent to those skilled in the art and are described in more detail in such publications as Remington's Pharmaceutical Science, 15th ed., Mack Publishing Company, Easton, Pa. (1980). In some instances, immunization is accomplished by intramuscular injection of an HCV immunogenic composition of the present disclosure.

In some cases, subject method comprises administering to an individual in need thereof an effective amount of a composition of the present disclosure that comprises an E2 polypeptide and/or an E1/E2 heterodimeric complex from two different HCV genotypes, as described hereinabove and below, where the composition comprises a pharmaceutically acceptable excipient.

In some cases, subject method comprises administering to an individual in need thereof an effective amount of a composition comprising: an E2 polypeptide of HCV genotype 1a; and an E1/E2 heterodimeric polypeptide complex of HCV genotype 3a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: a full-length E2 polypeptide of HCV genotype 1a; and an E1/E2 heterodimeric polypeptide complex of HCV genotype 3a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: a soluble E2 polypeptide of HCV genotype 1a; and an E1/E2 heterodimeric polypeptide complex of HCV genotype 3a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: an E2 polypeptide of HCV genotype 1a, where the E2 polypeptide comprises a modification (e.g., a substitution) of an asparagine at the E2N1 and/or E2N6 site such as described above; and an E1/E2 heterodimeric polypeptide complex of HCV genotype 3a, where the E2 polypeptide of the E1/E2 heterodimer is wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: an E2 polypeptide of HCV genotype 1a, where the E2 polypeptide is wild-type; and an E1/E2 heterodimeric polypeptide complex of HCV genotype 3a, where the E2 polypeptide of the E1/E2 heterodimer comprises a modification (e.g., a substitution) of an asparagine at the E2N1 and/or E2N6 site such as described above. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: an E2 polypeptide of HCV genotype 3a; and an E1/E2 heterodimeric polypeptide complex of HCV genotype 1a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: a full-length E2 polypeptide of HCV genotype 3a; and an E1/E2 heterodimeric polypeptide complex of HCV genotype 1a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: a soluble E2 polypeptide of HCV genotype 3a; and an E1/E2 heterodimeric polypeptide complex of HCV genotype 1a, where all of the polypeptides are wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: an E2 polypeptide of HCV genotype 3a, where the E2 polypeptide comprises a modification (e.g., a substitution) of an asparagine at the E2N1 and/or E2N6 site such as described above; and an E1/E2 heterodimeric polypeptide complex of HCV genotype 1a, where the E2 polypeptide of the E1/E2 heterodimer is wild-type. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: an E2 polypeptide of HCV genotype 3a, where the E2 polypeptide is wild-type; and an E1/E2 heterodimeric polypeptide complex of HCV genotype 1a, where the E2 polypeptide of the E1/E2 heterodimer comprises a modification (e.g., a substitution) of an asparagine at the E2N1 and/or E2N6 site such as described above. In some cases, the composition is administered in an amount that is effective to neutralize HCV genotypes most prevalent in North America and among i.v. drug users. In some cases, the composition is administered in an amount that is effective to neutralize HCV genotypes 1a and 3a. In some cases, the composition is administered in an amount that is effective to induce neutralizing antibody to HCV genotypes 1a and 3a. In some cases, the composition is administered in an amount that is effective to induce a CTL response to HCV genotypes 1a and 3a. In some cases, the composition is administered in an amount that is effective to achieve 50% or greater than 50% neutralization of HCV genotypes 1a and 3a. In some embodiments, the composition does not include an E1 polypeptide, an E2 polypeptide, and/or an E1/E2 polypeptide of any HCV genotype other than the above-mentioned genotypes 1a and 3a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition comprising: an E2 polypeptide or an E1/E2 heterodimeric polypeptide complex of HCV genotype 1a; and an E2 polypeptide or an E1/E2 heterodimeric polypeptide complex of HCV genotype 2a. In some cases, all of the polypeptides are wild-type. In some cases, the genotype 1a and the genotype 2a polypeptides are both E1/E2 heterodimeric polypeptide. In some cases, the genotype 1a polypeptide is an E2 polypeptide; and the genotype 2a polypeptide is an E1/E2 heterodimeric polypeptide complex. In some cases, the genotype 1a polypeptide is an E1/E2 heterodimeric polypeptide complex; and the genotype 2a polypeptide is an E2 polypeptide. In some cases, the genotype 1a and the genotype 2a polypeptides are both E2 polypeptides. In some cases, the genotype 1a and the genotype 2a polypeptides are both soluble E2 polypeptides. In some cases, the composition is administered in an amount that is effective to neutralize HCV genotypes such as genotypes 1a, 1b, 2a, 4, 5, and 6a. In some cases, the composition is administered in an amount that is effective to neutralize HCV genotypes 1a and 2a. In some cases, the composition is administered in an amount that is effective to induce neutralizing antibody to HCV genotypes 1a and 2a. In some cases, the composition is administered in an amount that is effective to induce a CTL response to HCV genotypes 1a and 2a. In some cases, the composition is administered in an amount that is effective to achieve 50% or greater than 50% neutralization of HCV genotypes 1a and 2a. In some cases, the composition is administered in an amount that is effective to achieve 50% or greater than 50% neutralization of HCV genotypes 1a, 1b, 2a, 4, 5, and 6a. In some embodiments, the composition does not include an E1 polypeptide, an E2 polypeptide, and/or an E1/E2 polypeptide of any HCV genotype other than the above-mentioned genotypes 1a and 2a. In some embodiments, a subject composition also includes an E1 polypeptide, and E2 polypeptide, and/or an E1/E2 polypeptide of at least one additional HCV genotype.

In some cases, subject method comprises administering to an individual in need thereof an effective amount of a composition of the present disclosure that comprises an E2 polypeptide and/or an E1/E2 heterodimeric complex from three different HCV genotypes, as described hereinabove and below, where the composition comprises a pharmaceutically acceptable excipient.

In some cases, subject method comprises administering to an individual in need thereof an effective amount of a composition comprising: an E2 polypeptide of HCV genotype 1a; an E1/E2 heterodimeric polypeptide complex of HCV genotype 2a; and an E2 polypeptide of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

In some cases, subject method comprises administering to an individual in need thereof an effective amount of a composition comprising: an E2 polypeptide of HCV genotype 1a; an E2 polypeptide of HCV genotype 2a; and an E1/E2 heterodimeric polypeptide complex of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

In some cases, subject method comprises administering to an individual in need thereof an effective amount of a composition comprising: an E1/E2 heterodimeric polypeptide complex of HCV genotype 1a; an E2 polypeptide of HCV genotype 2a; and an E2 polypeptide of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

In some cases, subject method comprises administering to an individual in need thereof an effective amount of a composition comprising: an E1/E2 heterodimeric polypeptide complex of HCV genotype 1a; an E1/E2 heterodimeric polypeptide complex of HCV genotype 2a; and an E2 polypeptide of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

In some cases, subject method comprises administering to an individual in need thereof an effective amount of a composition comprising: an E2 polypeptide of HCV genotype 1a; an E1/E2 heterodimeric polypeptide complex of HCV genotype 2a; and an E1/E2 heterodimeric polypeptide complex of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

In some cases, subject method comprises administering to an individual in need thereof an effective amount of a composition comprising: an E1/E2 heterodimeric polypeptide complex of HCV genotype 1a; an E2 polypeptide of HCV genotype 2a; and an E1/E2 heterodimeric polypeptide complex of HCV genotype 3a. In some cases, all of the polypeptides are wild-type. In other cases, the E2 polypeptide of the E1/E2 heterodimer comprises a modification (e.g., a substitution) of an asparagine at the E2N1 and/or E2N6 site such as described above. In other cases, the E2 polypeptide that is not in a heterodimeric complex with E1 comprises a modification (e.g., a substitution) of an asparagine at the E2N1 and/or E2N6 site such as described above. In some cases, at least one of the E2 polypeptides is full length. In some cases, at least one of the E2 polypeptides is a soluble E2 polypeptide. In some cases, the composition is administered in an amount that is effective to neutralize HCV genotypes prevalent globally. In some cases, the composition is administered in an amount that is effective to neutralize HCV genotypes 1a, 1b, 2a, 2b, 3a, 4a, 5a, 6a, and 7a. In some cases, the composition is administered in an amount that is effective to induce neutralizing antibody to HCV genotypes 1a, 1b, 2a, 2b, 3a, 4a, 5a, 6a, and 7a. In some cases, the composition is administered in an amount that is effective to induce a CTL response to HCV genotypes 1a, 1b, 2a, 2b, 3a, 4a, 5a, 6a, and 7a. In some cases, the composition is administered in an amount that is effective to achieve 50% or greater than 50% neutralization of HCV genotypes 1a, 1b, 2a, 2b, 3a, 4a, 5a, 6a, and 7a. In some embodiments, the composition does not include an E1 polypeptide, an E2 polypeptide, and/or an E1/E2 polypeptide of any HCV genotype other than the above-mentioned genotypes 1a, 2a, and 3a. In some embodiments, the composition also includes an E1 polypeptide, and E2 polypeptide, and/or an E1/E2 polypeptide of at least one additional HCV genotype. In some embodiments, the composition also includes an E1 polypeptide, and E2 polypeptide, and/or an E1/E2 polypeptide of HCV genotype 2b. In some embodiments, the composition also includes an E1 polypeptide, and E2 polypeptide, and/or an E1/E2 polypeptide of HCV genotype 7a.

### Individuals Suitable for Administration

Individuals who are suitable for administration with an HCV composition of the present disclosure include immunologically naïve individuals (e.g., individuals who have not been infected with HCV and/or who have not been administered with an HCV vaccine).

Individuals who are suitable for administration with an HCV composition of the present disclosure include individuals who are at greater risk than the general population of becoming infected with HCV, where such individuals include, e.g., intravenous drug users; individuals who are the recipients, or the prospective recipients, of blood or blood products from another (donor) individual(s); individuals who are the recipients, or the prospective recipients, of non-autologous cells, tissues, or organs from another (donor) individual; health care workers; emergency medical and non-medical personnel (e.g., first responders; fire fighters; emergency medical team personnel; etc.) and the like.

Individuals who are suitable for administration with an HCV composition of the present disclosure include individuals who recently became exposed to HCV or who recently became infected with HCV. For example, a subject immunogenic composition can be administered to an individual within from about 24 hours to about 48 hours, from about 48 hours to about 1 week, or from about 1 week to about 4 weeks, following possible or suspected exposure to HCV or following infection with HCV.

Individuals who are suitable for administration with an HCV composition of the present disclosure include individuals who have been diagnosed as having an HCV infection, and include chronically infected individuals. In some cases, an individual who has been diagnosed as having an HCV infection is treated with an anti-viral agent and a subject HCV immunogenic composition. Suitable anti-viral agents for treating HCV infection include, e.g., ribavirin (1-f3-D-ribofuranosyl-1H-1,2,4-triazole-3-carboxamide); interferon-alpha (IFN-α) (where “IFN-α” includes IFN-α2a; IFN-α2b; IFN-α that is conjugated with poly(ethylene glycol) (“pegylated IFN-α), where the pegylated IFN-α can be pegylated IFN-α2a or pegylated IFN-α 2b); an HCV NS3 protease inhibitor (e.g., who has been diagnosed as having an HCV infection is treated with, e.g.: 1) IFN-α+ribavirin; and a subject HCV immunogenic composition; or 2) IFN-α+ribavirin+an HCV protease inhibitor (e.g., boceprevir or telaprevir); and a subject HCV immunogenic composition. As one non-limiting example, and a subject HCV immunogenic composition is administered to an individual who has been diagnosed as having an HCV infection once a month for 6 months.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Genotype-Specific Neutralization

### Materials and Methods

### Goat Immunization

Goats (G714 and G757) and Goats (G766 and G773) were immunized with HCV1 (Genotype 1a)-derived E1E2 and with J6 (genotype 2a)-derived E2, respectively. Recombinant E1E2 and soluble E2 proteins were derived from the sequence of an HCV genotype 1a strain and HCV genotype 2a strain respectively. The recombinant E1E2 proteins were purified from Chinese hamster ovary cell line constitutively expressing the glycoproteins; and the E2 protein was purified from culture medium of 293T cells expressing the E2 glycoprotein. 0.5 ml experimental vaccine, which was composed of 10 μg of purified protein antigen (E2E1 or E2) along with either squalene-based Addavax or Freund's adjuvant were administered to goat. Post-vaccinated serum used in this data was collected after four vaccinations at 129 days post-immunization. Serum collected prior to vaccination was used as a negative control.

### In Vitro Neutralization Assay

Post-vaccinated goat sera were tested for neutralization activity against chimeric genotype 1a H77c/JFH1 or genotype 2a J6/JFH1 HCV produced in cell culture (HCVcc) in vitro.

1×10⁴ Huh-7.5 cells per well were seeded in a multi-well plate one day prior to infection. For infection, 300 TCID50 (median tissue culture infectious dose) HCVcc were premixed with heat-inactivated goat sera diluted at 1 in 50 (by volume), for 1 hour at 37° C. The pre-mixed HCVcc/goat sera were then added to cells. 12 hour post-infection, the antibody-virus inoculum was replaced with fresh culture media. Cells were then fixed 48 hours post-infection with 2% paraformaldehyde. The amount of infection was quantitated by flow cytometry, counting the number of NS5A-positive cells detected using mouse monoclonal NS5A antibody (9E10). The percentage of neutralization was reported by comparison with pre-vaccination serum.

### Results

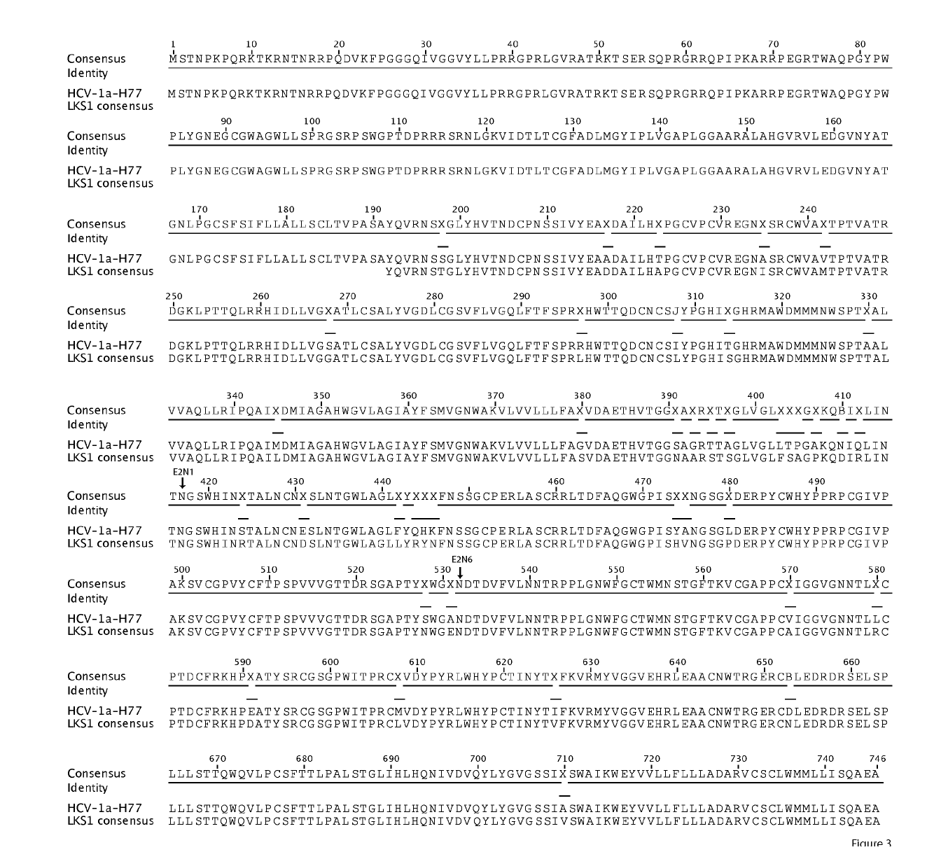

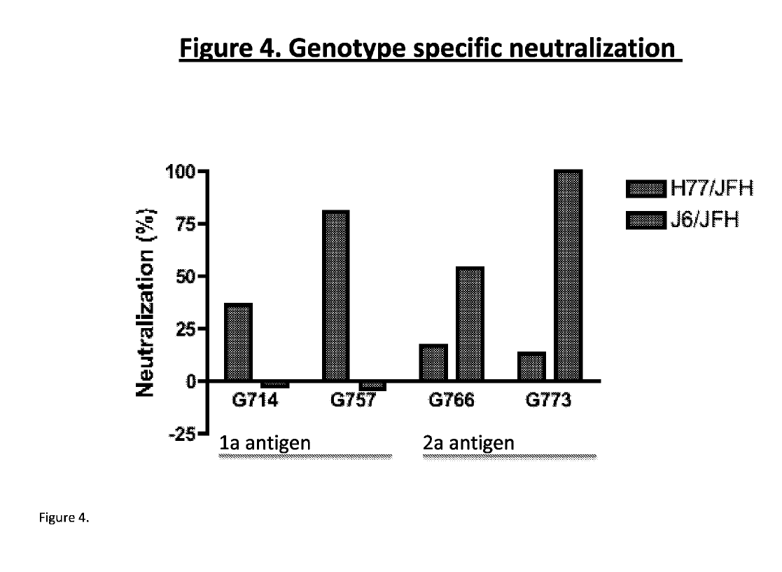

The neutralization activity of post-vaccinated goat serum was tested against tissue culture-derived chimeric genotype 1a (H77c/JFH1) or 2a (J6/JFH1) using an in vitro neutralization assay. Goats (G714 and G757) were immunized with recombinant E1E2 derived from sequence of genotype 1a (G1a), whereas goats (G766 and G773) were immunized with E2 derived from sequence of genotype 2a (G2a). The G1a antigen induced strong neutralization activity against G1a chimeric virus H77/JFH, but showed a reduced efficiency against G2a virus J6/JFH (FIG. 4). On the other hand, the E2 antigen derived from G2a induced more effective neutralizing antibodies against G2a virus than G1a virus. Together, the data indicate the need for an antigen cocktail to confer cross-genotype protection in a global vaccine.

### Example 2

### Genotype-Specific Neutralization

### Materials and Methods

### Goat Immunization

Goats (G757, G714) were immunized with HCV1 (Genotype 1a)-derived E1E2; Goats (G004, G752) were immunized with HCV1 (Genotype 1a)-derived ectodomain of E1; Goats (G786, G799) were immunized with HCV1 (Genotype 1a)-derived ectodomain of E2; Goats (G766, G733) were immunized with J6 (Genotype 2a)-derived ectodomain of E2, respectively. E1E2 proteins, ectodomain of E1 and E2 were derived from the sequence of an HCV genotype 1a (H77) strain and HCV genotype 2a (J6) strain, respectively. The recombinant antigens were purified from Chinese hamster ovary cell line constitutively expressing the glycoproteins. 0.5 ml experimental vaccine, which was composed of 10 μg of purified protein antigen (E1E2) or the molar equivalent of E1 or E2 along with either squalene-based AddaVax or Incomplete Freund's Adjuvant (IFA) were administered to goat. Post-vaccinated serum used in this data was collected after four vaccinations at 129 days post-immunization. Serum collected prior to vaccination was used as a negative control. Ectodomain of E1 and ectodomain of E2 are soluble E1 and E2 polypeptides with the C-terminal transmembrane regions removed.

### In Vitro Neutralization Assay

Post-vaccinated goat sera were tested for neutralization activity against chimeric genotype 1a H77c/JFH1 or genotype 2a J6/JFH1 HCV produced in cell culture (HCVcc) in vitro.

1×10⁴ Huh-7.5 cells per well were seeded in a multi-well plate one day prior to infection. For infection, 300 TCID50 (median tissue culture infectious dose) HCVcc were premixed with heat-inactivated goat sera diluted at 1 in 50 (by volume), for 1 hour at 37° C. The pre-mixed HCVcc/goat sera were then added to cells. 12 hour post-infection, the antibody-virus inoculum was replaced with fresh culture media. Cells were then fixed 48 hours post-infection with 2% paraformaldehyde. The amount of infection was quantitated by flow cytometry, counting the number of NS5A-positive cells detected using mouse monoclonal NS5A antibody (9E10). The percentage of neutralization was reported by comparison with pre-vaccination serum.

### Results

Cross neutralization of vaccine-induced antibody was tested. Sera diluted 1:50 were pre-incubated with either tissue culture-derived HCV of H77/JFH1 (G1a) or J6/JFH (G2a), then used to infect Huh-7.5 cells. The level of infection was monitored by NS5a staining using flow cytometry. The level of neutralization activity was normalized with one set of pre-vaccinated control to 0%. A second set of pre-vaccination sera was used to show the variation of the assay.

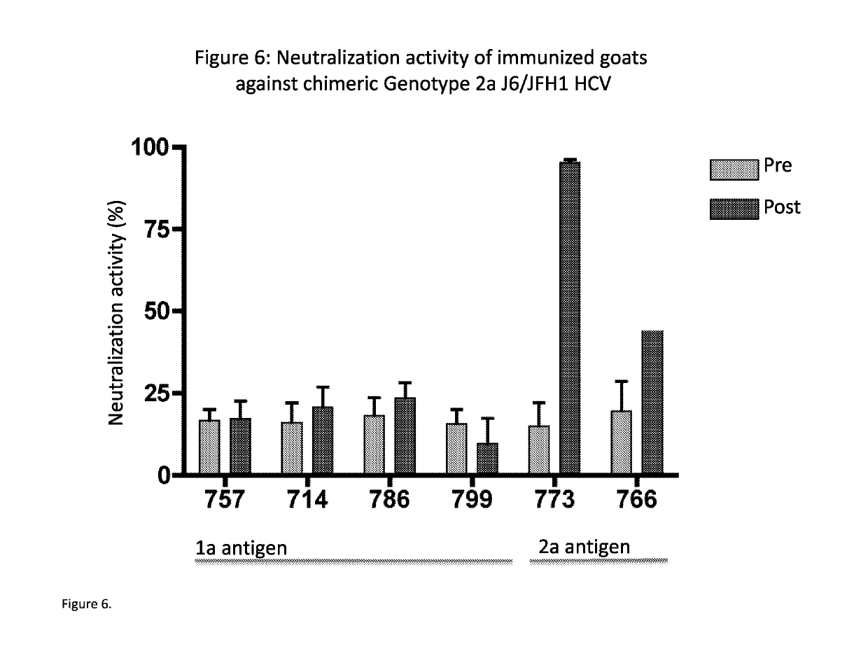

The results are shown in FIGS. 5 and 6. In FIG. 5, the ability of goat sera to neutralize HCV genotype 1a (HCV of H77/JFH1 as the “challenge virus”) is shown. In FIG. 6, the ability of goat sera to neutralize HCV genotype 2a (HCV of J6/JFH as the “challenge virus”) is shown.

FIG. 5. The sera of goats 757, 714, 786, and 799 (immunized with HCV1 (G1a) derived antigen) showed induction of neutralizing antibodies to block G1a infection; however, the sera of goats 766 and 773 (immunized with J6 (G2a derived antigen) had limited effectiveness in blocking infection of G1a (genotype 1a) virus.

FIG. 6. The sera of goats 766 and 733 (immunized with antigen derived from genotype 2a) exhibited greater blocking of infection of G2a (genotype 2a) virus than of G1a virus. Sera from Goats (757, 714, 004, 752, 786, 799), immunized with antigen derived from genotype 1a, have limited neutralization activity against G2a virus.

### Example 3

### Cross-Neutralization

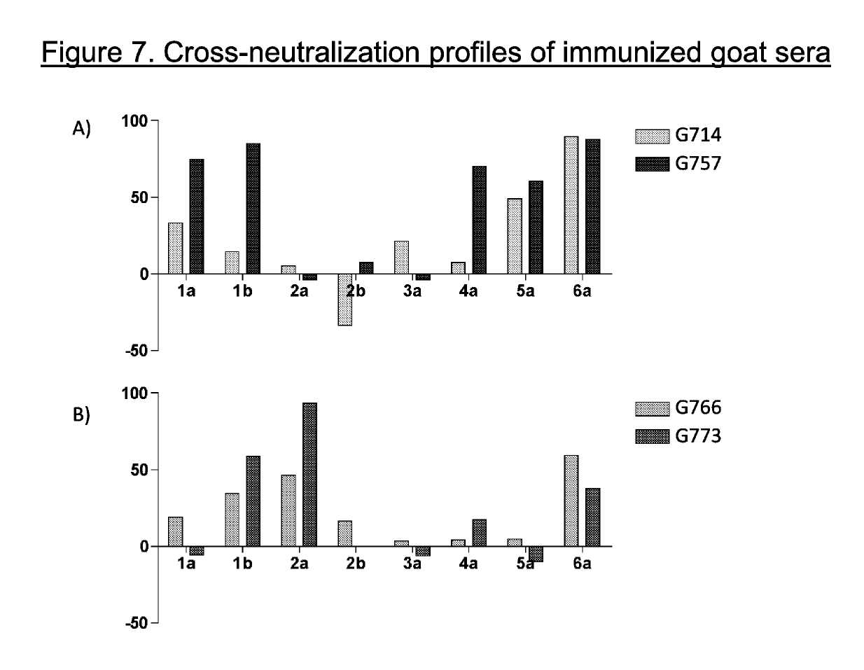

Goats were immunized with E1/E2 heterodimeric complex from HCV strain HCV1 (genotype 1a) or with soluble E2 (sE2) from HCV strain J6 (genotype 2a). Post-vaccinated goat sera were tested for neutralization activity against HCV genotype 1a, 1b, 2a, 2b, 3a, 4a, 5a, and 6a. The data are shown in FIGS. 7A and 7B. The data presented in FIG. 7a show that immunization with E1/E2 from HCV genotype 1a elicited neutralizing antibodies against HCV of all genotypes tested except 2a, 2b, and 3a; and FIG. 7b show that immunization with sE2 from HCV genotype 2a elicited neutralizing antibodies against HCV of genotypes 1b, 2a, 4a, and 6a.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An immunogenic composition comprising:
a) an hepatitis C virus (HCV) E1 polypeptide, E2 polypeptide or E1E2 polypeptide from a first HCV genotype;
b) an HCV E1 polypeptide, E2 polypeptide, or E1E2 polypeptide from a second HCV genotype; and
c) a pharmaceutically acceptable excipient,
with the proviso that the composition comprises at least one E1 polypeptide and at least one E2 polypeptide.

2. The composition of claim 1, wherein the first HCV genotype is genotype 1; and
the second HCV genotype is genotype 2.

3. The composition of claim 1, wherein the first HCV genotype is genotype 1; and
the second HCV genotype is genotype 3.

4. The composition of claim 1, wherein composition comprises:
i) an E1/E2 heterodimeric polypeptide complex of HCV genotype 1; and E1/E2 polypeptide of HCV genotype 3;
ii) an E1/E2 heterodimeric polypeptide complex of HCV genotype 1; and an E2 polypeptide of HCV genotype 3;
iii) an E2 polypeptide of HCV genotype 1; and an E1/E2 heterodimeric polypeptide complex of HCV genotype 3;
iv) an E1/E2 heterodimeric polypeptide complex of HCV genotype 1; and an E1/E2 heterodimeric polypeptide complex of HCV genotype 2;
v) an E1/E2 heterodimeric polypeptide complex of HCV genotype 1; and an E2 polypeptide of HCV genotype 2;
vi) an E2 polypeptide of HCV genotype 1; and an E1/E2 heterodimeric polypeptide complex of HCV genotype 2;
vii) an E1 polypeptide of HCV genotype 1; and an E2 polypeptide of HCV genotype 3;
viii) an E1 polypeptide of HCV genotype 1; and an E1/E2 heterodimeric polypeptide complex of HCV genotype 3;
ix) an E2 polypeptide of HCV genotype 1; and an E1 polypeptide of HCV genotype 3;
x) an E2 polypeptide of HCV genotype 1; and an E1/E2 heterodimeric polypeptide complex of HCV genotype 3;
xi) an E1 polypeptide of HCV genotype 1; and an E2 polypeptide of HCV genotype 2;
xii) an E1 polypeptide of HCV genotype 1; and an E1/E2 heterodimeric polypeptide complex of HCV genotype 2;
xiii) an E2 polypeptide of HCV genotype 1; and an E1 polypeptide of HCV genotype 2; or
xiv) an E2 polypeptide of HCV genotype 1; and an E1/E2 heterodimeric polypeptide complex of HCV genotype 2.

5. The composition of claim 4, further comprising an HCV E1, E2, or E1/E2 polypeptide of a third HCV genotype.

6. The composition of claim 1, wherein HCV E2 polypeptide from the first HCV genotype is wild-type, and wherein the HCV E2 polypeptide from the second HCV genotype is wild-type.

7. The composition of claim 1, wherein HCV E2 polypeptide from the first HCV genotype is wild-type, and wherein the HCV E2 polypeptide from the second HCV genotype comprises an amino acid substitution of asparagine in an E2N1 site and/or an E2N6 site.

8. The composition of claim 1, wherein HCV E2 polypeptide from the first HCV genotype comprises an amino acid substitution of asparagine in an E2N1 site and/or an E2N6 site, and wherein the HCV E2 polypeptide from the second HCV genotype is wild-type.

9. The composition of claim 1, wherein the pharmaceutically acceptable excipient comprises an adjuvant.

10. The composition of claim 1, wherein the adjuvant is MF59, alum, poly(DL-lactide co-glycolide), or a CpG oligonucleotide.

11. An immunogenic composition comprising:
a) a hepatitis C virus (HCV) E1 polypeptide, E2 polypeptide, or E1/E2 polypeptide from a first HCV genotype;
b) an HCV E1 polypeptide, E2 polypeptide, or E1/E2 polypeptide from a second HCV genotype;
c) an HCV E1 polypeptide, E2 polypeptide, or E1/E2 polypeptide from a third HCV genotype; and
d) a pharmaceutically acceptable excipient,
with the proviso that the composition comprises at least one E1 polypeptide and at least one E2 polypeptide.

12. The immunogenic composition of claim 11, wherein the first HCV genotype is genotype 1, wherein the second HCV genotype is genotype 2, and wherein the third HCV genotype is genotype 3.

13. The immunogenic composition of claim 11, wherein the composition comprises:
i) an E1/E2 heterodimeric polypeptide complex of HCV genotype 1; an E1/E2 heterodimeric polypeptide complex of HCV genotype 2; and an E1/E2 heterodimeric polypeptide complex of HCV genotype 3;
ii) an E1/E2 heterodimeric polypeptide complex of HCV genotype 1; an E1/E2 heterodimeric polypeptide complex of HCV genotype 2; and an E2 polypeptide of HCV genotype 3;
iii) an E1/E2 heterodimeric polypeptide complex of HCV genotype 1; an E2 polypeptide of HCV genotype 2; and an E1/E2 heterodimeric polypeptide complex of HCV genotype 3;
iv) an E2 polypeptide of HCV genotype 1; an E1/E2 heterodimeric polypeptide complex of HCV genotype 2; and an E1/E2 heterodimeric polypeptide complex of HCV genotype 3;
v) an E1/E2 heterodimeric polypeptide complex of HCV genotype 1; an E2 polypeptide of HCV genotype 2; and an E2 polypeptide of HCV genotype 3;
vi) an E2 polypeptide of HCV genotype 1; an E1/E2 heterodimeric polypeptide complex of HCV genotype 2; and an E2 polypeptide of HCV genotype 3;
vii) an E2 polypeptide of HCV genotype 1; an E2 polypeptide of HCV genotype 2; and an E1/E2 heterodimeric polypeptide complex of HCV genotype 3;
viii) an E1 polypeptide of HCV genotype 1; an E1/E2 heterodimeric polypeptide complex of HCV genotype 2; and an E1/E2 heterodimeric polypeptide complex of HCV genotype 3;
ix) an E1/E2 heterodimeric polypeptide complex of HCV genotype 1; an E1 polypeptide of HCV genotype 2; and an E1/E2 heterodimeric polypeptide complex of HCV genotype 3;
x) an E1/E2 heterodimeric polypeptide complex of HCV genotype 1; an E1/E2 heterodimeric polypeptide complex of HCV genotype 2; and an E1 polypeptide of HCV genotype 3;
xi) an E1 polypeptide of HCV genotype 1; an E2 polypeptide of HCV genotype 2; and an E1/E2 heterodimeric polypeptide complex of HCV genotype 3;
xii) an E2 polypeptide of HCV genotype 1; an E1 polypeptide of HCV genotype 2; and an E1/E2 heterodimeric polypeptide complex of HCV genotype 3;
xiii) an E1/E2 heterodimeric polypeptide complex of HCV genotype 1; an E2 polypeptide of HCV genotype 2; and an E1 polypeptide of HCV genotype 3;
xiv) an E1 polypeptide of HCV genotype 1; an E2 polypeptide of HCV genotype 2; and an E2 polypeptide of HCV genotype 3;
xv) an E2 polypeptide of HCV genotype 1; an E1 polypeptide of HCV genotype 2; and an E2 polypeptide of HCV genotype 3;
xvi) an E2 polypeptide of HCV genotype 1; an E2 polypeptide of HCV genotype 2; and an E1 polypeptide of HCV genotype 3; or
xvii) an E1 polypeptide of HCV genotype 1; an E1/E2 heterodimeric polypeptide complex of HCV genotype 2; and an E2 polypeptide of HCV genotype 3.

14. The composition of claim 11, wherein HCV E2 polypeptide from the first HCV genotype is wild-type, wherein the HCV E2 polypeptide from the second HCV genotype is wild-type, and wherein the HCV E2 polypeptide from the third HCV genotype is wild-type.

15. The composition of claim 11, wherein HCV E2 polypeptide from the first HCV genotype is wild-type, wherein the HCV E2 polypeptide from the second HCV genotype is wild-type, and wherein the HCV E2 polypeptide from the third HCV genotype comprises an amino acid substitution of asparagine in an E2N1 site and/or an E2N6 site.

16. The composition of claim 11, wherein HCV E2 polypeptide from the first HCV genotype is wild-type, wherein the HCV E2 polypeptide from the second HCV genotype comprises an amino acid substitution of asparagine in an E2N1 site and/or an E2N6 site, and wherein the HCV E2 polypeptide from the third HCV genotype is wild-type.

17. The composition of claim 11, wherein HCV E2 polypeptide from the first HCV genotype comprises an amino acid substitution of asparagine in an E2N1 site and/or an E2N6 site, wherein the HCV E2 polypeptide from the second HCV genotype is wild-type, and wherein the HCV E2 polypeptide from the third HCV genotype is wild-type.

18. The composition of claim 11, wherein the pharmaceutically acceptable excipient comprises an adjuvant.

19. The composition of claim 11, wherein the adjuvant is MF59, alum, poly(DL-lactide co-glycolide), or a CpG oligonucleotide.

20. A method of inducing an immune response in an individual, the method comprising administering to the individual an effective amount of the composition of any of claims 1 to 19.

21. A composition comprising:
a) a hepatitis C virus (HCV) E2 polypeptide of HCV genotype 1a;
b) an HCV E1/E2 heterodimeric polypeptide of HCV genotype 3a; and
c) a pharmaceutically acceptable excipient.

22. The composition of claim 21, wherein all of the polypeptides are wild-type.

23. The composition of claim 21, wherein the E2 polypeptide is full-length.

24. The composition of claim 21, wherein the E2 polypeptide is a soluble E2 polypeptide.

25. A composition comprising:
a) a hepatitis C virus (HCV) E2 polypeptide or an HCV E1/E2 heterodimeric polypeptide of HCV genotype 3a;
b) an HCV E2 polypeptide or an HCV E1/E2 heterodimeric polypeptide of HCV genotype 1a; and
c) a pharmaceutically acceptable excipient.

26. The composition of claim 25, wherein all of the polypeptides are wild-type.

27. The composition of claim 25, wherein the E2 polypeptide is full-length.

28. The composition of claim 25, wherein the E2 polypeptide is a soluble E2 polypeptide.

29. A method of inducing an immune response in an individual, the method comprising administering to the individual an effective amount of the composition of any one of claims 21-28, wherein the composition is administered in an amount effective to induce neutralizing antibody to at least HCV genotypes 1a and 3a.

30. A method of inducing an immune response in an individual, the method comprising administering to the individual an effective amount of the composition of any one of claims 21-28, wherein the composition is administered in an amount effective to induce a cytotoxic T lymphocyte response to at least HCV genotypes 1a and 3a.

31. A method of inducing an immune response in an individual, the method comprising administering to the individual an effective amount of the composition of any one of claims 21-28, wherein the composition is administered in an amount effective to achieve 50% or greater neutralization of HCV genotypes 1a and 3a.

32. A composition comprising:
a) a hepatitis C virus (HCV) E2 polypeptide or an HCV E1/E2 heterodimeric polypeptide of HCV genotype 2a;
b) an HCV E2 polypeptide or an HCV E1/E2 heterodimeric polypeptide of HCV genotype 1a; and
c) a pharmaceutically acceptable excipient.

33. The composition of claim 32, wherein all of the polypeptides are wild-type.

34. The composition of claim 32, wherein the E2 polypeptide is full-length.

35. The composition of claim 32, wherein the E2 polypeptide is a soluble E2 polypeptide.

36. A method of inducing an immune response in an individual, the method comprising administering to the individual an effective amount of the composition of any one of claims 32-35, wherein the composition is administered in an amount effective to induce neutralizing antibody to at least HCV genotypes 1a and 2a.

37. A method of inducing an immune response in an individual, the method comprising administering to the individual an effective amount of the composition of any one of claims 32-35, wherein the composition is administered in an amount effective to induce a cytotoxic T lymphocyte response to at least HCV genotypes 1a and 2a.

38. A method of inducing an immune response in an individual, the method comprising administering to the individual an effective amount of the composition of any one of claims 32-35, wherein the composition is administered in an amount effective to achieve 50% or greater neutralization of HCV genotypes 1a and 2a.

39. A composition comprising:
a) a hepatitis C virus (HCV) E2 or an HCV E1/E2 heterodimeric polypeptide of HCV genotype 1a;
b) an HCV E2 polypeptide or an HCV E1/E2 heterodimeric polypeptide of HCV genotype 2a;
c) an E2 polypeptide or an HCV E1/E2 heterodimeric polypeptide of HCV genotype 3a; and
d) a pharmaceutically acceptable excipient.

40. The composition of claim 39, wherein all of the polypeptides are wild-type.

41. The composition of claim 39, wherein the at least one of the E2 polypeptides are full-length.

42. The composition of claim 39, wherein at least one of the E2 polypeptides is a soluble E2 polypeptide.

43. The composition of any one of claims 39-42, comprising an E1/E2 polypeptide of HCV genotype 7a.

44. The composition of any one of claims 39-42, comprising an E2 polypeptide of HCV genotype 7a.

45. A method of inducing an immune response in an individual, the method comprising administering to the individual an effective amount of the composition of any one of claims 39-44, wherein the composition is administered in an amount effective to induce neutralizing antibody to HCV genotypes 1a, 1b, 2a, 2b, 3a, 4a, 5a, 6a, and 7a.

46. A method of inducing an immune response in an individual, the method comprising administering to the individual an effective amount of the composition of any one of claims 39-44, wherein the composition is administered in an amount effective to induce a cytotoxic T lymphocyte response to HCV genotypes 1a, 1b, 2a, 2b, 3a, 4a, 5a, 6a, and 7a.

47. A method of inducing an immune response in an individual, the method comprising administering to the individual an effective amount of the composition of any one of claims 39-44, wherein the composition is administered in an amount effective to achieve 50% or greater neutralization of HCV genotypes 1a, 1b, 2a, 2b, 3a, 4a, 5a, 6a, and 7a.


SEQUENCE LISTING (53 sequences)

SEQ ID NO: 1 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 2 - protein, 746 aa, Hepatitis C virus
MSTNPKPQRKTKRNTNRRPQDVKFPGGGQIVGGVYLLPRRGPRLGVRAVRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGKLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAETYTTGGSVAQAAFGLTSLFRPGPKQDIQLINTNGSWHINRTALNCNASLDTGWVAGLFYYHKFNSSGCPERMASCRSLADFDQGWGPISYANGSGPEHRPYCWHYPPKPCGIVPAQNVCGPVYCFTPSPVVVGTTNKLGVPTYSWGSNETDVLVLNNTRPPLGNWFGCTWMNSSGFTKVCGAPPCVIGGAGNRTLHCPTDCFRKHPEATYSRCGSGPWITPRCLVHYPYRLWHYPCTVNYTMFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 3 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAFQVRNSSGLYHVTNDCPNSSIVYETADTILHSPGCVPCVREDNTSRCWVAVAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFASVDAETYTSGGSVARATAGFAGIFNPGAKQDIQLINTNGSWHINRTALNCNASLDTGWVAGLFYYHKFNSSGCPERMASCKPLAHFAQGWGPISYANGSGPDHRPYCWHYPPKPCGIVPAQNVCGPVYCFTPSPVVVGTTNKLGAPTYNWGSNDTDVFILNNTRPPGGNWFGCTWMNSSGFTKVCGAPPCTIGGVGNNTLLCPTDCFRKHPEATYSRCGSGPWVTPRFLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSLVSWAIKWEYVILLFLLLADARICSCLWMMLLISQAEA

SEQ ID NO: 4 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNTSKCWVAVAPTVATRDGKLPTTQLRRHIDLLVGSATLCSALYVGDLCGSVFLVGQLFTFSPRRHWTTQDCNCSIYPGHITGHRMAWDMMMNWSPTAALVTAQLLRVPQAILDMIAGAHWGVLAGIAYFSMAGNWAKVLLVLLLFAGVDAETYTTGGSVARTTRGLASLLQVGPKQDIRLIHTNGSWHINRTALNCNASLDTGWLAGLLYYHKFNSSGCPERMASCRPLADFDQGWGPISYANGSGPEHRPYCWHYPPKPCGIVPAQTVCGPVYCFTPSPVVVGTTNKLGVPTYTWGSNDTDVFVLNNTRPPLGNWFGCTWMNSTGFTKVCGAPPCVIGGVGNNTLHCPTDCFRKHPEATYSRCGSGPWITPRCLVDYPYRLWHYPCTVNYTLFKVRMYVGGVEHRLQAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 5 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREDNASRCWVPVAPTVATRDGKLPTTQLRRHIDLLVGSATLCSALYVGDLCGSVFLVSQLFTFSPRRHWTTQDCNCSIYPGHITGHRMAWDMMMNWSPTVALVMAQLLRVPQAILDMIAGAHWGVLAGIAYFSMVGNWAKVLVVLLLFAGVDAQTYVTGGSAARGASGLANLFTPGAKQDIQLINTNGSWHINRTALNCNASLDTGWVAGLFYYHKFNSSGCPERMASCKPLADFDQGWGPIRHANGSGPEHRPYCWHYPPKPCGIVSAQTVCGPVYCFTPSPVVVGTTNRLGVPTYSWGTNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 6 - protein, 746 aa, Hepatitis C virus
MSTNPKPQRKTKRNTNRRPQDVKFPGGGQIVGGVYLLPRRGPRLGVRTTRKTSERSEPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETKDTILHSPGCVPCVREGNVSKCWVPVALTVATRDGNLPTTQLRRHIDLLVGSATLCSALYVGDLCGSVFLVGQLFTFSPRRHWTTQDCNCSMYPGHITGHRMAWDMMMNWSPTSALVVAQLLRVPQAILDMIAGAHWGVLAGLAYFSMVGNWAKVLVVLLLFASVDAGTHVTGGSAAHDVSALAGFFRRGAKQNIQLINTNGSWHVNRTALNCNASLDTGWVAGLLYYHRFNSSGCPERMASCRPLADFDQGWGPITNVDGGGSEYRPYCWHYPPKPCGIEPAQNVCGPVYCFTPSPVVVGTTDKVGVPTYNWGENDTDVFVLNNTRPPLGNWFGCTWMNSSGFVKVCGAPPCIIGGAGNKTLHCPTDCFRKHPDATYSRCGSGPWLTPRCLVDYPYRLWHYPCTVNYTLFKIRMYVGGVEHRLVAACNWTYGERCNLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 7 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGTLPTTQLRRHIDLLVGGATLCSALYVGDLCGSVFLVGQLFTFSPRRHWTTQDCNCSIYPGHITGHRMAWDMMMNWSPTTALIVAQLLRVPQAILDMIAGAHWGVLAGIAYFSMVGNWAKVLVVLLLFAGVDAETHVTGGSAAQVTSRVAGFFNPGPKQNVQLINTNGSWHINRTALNCNASLDTGWVAGLFYHYNFNSSGCPERMASCRPLADFDQGWGPISYANGSGPEHRPYCWHYPPKPCGIVPAQNVCGPVYCFTPSPVVVGTTNRLGVPTYNWGSNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 8 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRIPQAILDMIAGAHWGVLAGIAYFSMVGNWAKVLVVLLLFAGVDAHTRVTGGSAARATARLTTLFSPGAKQDIQLINTNGSWHINRTALNCNASLDTGWVAGLFYYHKFNSSGCPERMASCRPLADFDQGWGPISYANGSGPEHRPYCWHYPPKPCGIIPAKTVCGPVYCFTPSPVVVGTTDRSGAPTFNWGDNDTDVLVLNNTRPPLGNWFGCTWMNSSGYTKVCGAPPCIIGGVGNNTLHCPTDCFRKHPEATYSRCGSGPWITPRCLVHYPYRLWHYPCTVNYTLFKVRMYVGGVEHRLEAACNWTRGERCNLDDRDRSELSPLLLSTTQWQVLPCSFTTLPALTTGLIHLHQNIVDVQYLYGVGSSIVSWAIKWEYVILLFLLLADARVCSCLWMMLLISQVEA

SEQ ID NO: 9 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVTPTVATRDGRLPATQLRRHIDLLVGSATLCSALYVGDLCGSIFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAGTHVTGGSAAKDTSGFTSLFRIGARQNIQLINSNGSWHINRTALNCNESLDTGWVAGLLYYHKFNSSGCPERMASCRSLADFDQGWGPISYANGSGPEHRPYCWHYPPKPCGIVPAQSVCGPVYCFTPSPVVVGTTDKSGAPTYNWGCNETDVFVLNNTRPPLGNWFGCTWMNSSGFTKVCGAPPCVIGGVGNNTLHCPTDCFRKHPEATYSRCGSGPWITPRCLVHYAYRLWHYPCTVNYTLFKVRMYVGGVEHRLDVACNWTRGERCNLDDRDRSELSPLLLSTTQWQVLPCSFTTLPALSTGLIHLHQNIVDVQYLYGVGSSIVSWAIKWEYVILLFLLLADARICSCLWMMLLISQAEA

SEQ ID NO: 10 - protein, 746 aa, Hepatitis C virus; identical to SEQ ID NO: 9

SEQ ID NO: 11 - protein, 746 aa, Hepatitis C virus; identical to SEQ ID NO: 9

SEQ ID NO: 12 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADSILHSPGCVPCVREGNASKCWVAVAPTVATRDGKLPATQLRRHIDLLVGSATLCSALYVGDLCGSVFLVSQLFTFSPRRHWTTQDCNCSIYPGHITGHRMAWDMMMNWSPTTALVVSQLLRVPQAILDMIAGAHWGVLAGIAYFSMVGNWAKVLVVLLLFAGVDAETHTTGGSAAYATSGFVGLFRQGAKQNIQLINTNGSWHVNRTALNCNASLDTGWVAGLFYYHKFNSSGCPERLASCKPLANFDQGWGSISYTNGSGPEHRPYCWHYPPKPCGIVPAQNVCGPVYCFTPSPVVVGTTDRLGAPTFNWGENESDVFVLNNTRPPSGNWFGCTWMNSSGFTKVCGAPPCNIGGVGNNTLHCPTDCFRKHPEATYSRCGSGPWVTPRCLVHYPYRLWHYPCTLNYSLFKVRMYVGGIEHRLEVACNWTRGERCNLDDRDRSELSPLLLTTTQWQVLPCSFTTLPALTTGLIHLHQNVVDVQYLYGVGSSIVSWAIKWEYVILLFLLLADARICSCLWMMLLISQAEA

SEQ ID NO: 13 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGRLPTTQLRRHIDLLVGSATLCSALYVGDLCGSVFLVGQLFTFSPRHHWTTQDCNCSIYPGHITGHRMAWDMMMNWSPTTALVVAQLLRVPQAILDMIAGAHWGVLAGIAYFSMVGNWAKVLVVLLLFAGVDATTHTTGGAVAHNTRMFTSIFSLGPRQEIQLVNTNGSWHINRTALNCNASLETGWIAGLLYANRFNSSGCPERMASCKPLADFDQGWGPISYANGSGPEHRPYCWHYPPKPCGIVPAQNVCGPVYCFTPSPVVVGTTDRLGTPTYDWGSNDTDVFVLNNTRPPAGNWFGCTWMNSSGYTKVCGAPPCVIGGVSNNTLHCPTDCFRKHPEATYSRCGSGPWITPRCLVHYAYRLWHYPCTVNYTLFKVRMYVGGVEHRLEVACNWTRGERCNLDDRDRSELSPLLLSTTQWQVLPCSFTTLPALTTGLIHLHQNIVDVQYLYGVGSSIVSWAVKWEYVILLFLLLADARICSCLWMMLLISQAEA

SEQ ID NO: 14 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNASIVYEAADMIMHTPGCVPCVRENNSSRCWVALTPTLAARNASVPTTTIRRHVDLLVGAAALCSAMYVGDLCGSVFLVAQLFTFSPRRHETVQDCNCSIYPGHVTGHRMAWDMMMNWSPTAALVVSQLLRIPQAVVDMVAGAHWGVLAGLAYYSMVGNWAKVLIVMLLFAGVDGGTYVTGGTMAKNTLGITSLFSPGSSQKIQLVNTNGSWHINRTALNCNDSLNTGFLAALFYVHKFNSSGCPERMASCSPIDAFAQGWGPITYNESHSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGIGNKTLTCPTDCFRKHPEATYTKCGSGPWLTPRCLVHYPYRLWHYPCTVNFTIFKVRMYVGGVEHRLEAACNWTRGERCNLEDRDRSELSPLLLSTTEWQVLPCSFTTLPALSTGLIHLHQNVVDVQYLYGIGSAVVSFAIKWEYVLLLFLLLADARVCACLWMMLLIAQAEA

SEQ ID NO: 15 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGVARALAHGVRVVEDGVNYATGNLPGCSFSIFLLALLSCLTIPASAYEVRNVSGIYHVTNDCSNSSIVYEAADMIMHTPGCVPCVREGNSSRCWVALTPTLAARNASVPTTAIRRHVDLLVGAAAFCSAMYVGDLCGSVFLVSQLFTFSPRRHETIQDCNCSIYPGHVSGHRMAWDMMMNWSPTTALVVSQLLRIPQAIVDMVAGAHWGVLAGLAYYSMVGNWAKVLIVMLLFAGVDGETRVTGGQIARNAYSLTTLFSSGSAQNIQLINTNGSWHINRTALNCNDSLNTGFLAALFYTHKFNASGCPERLASCRPIDKFDQGWGPITYAEQGGQDQRPYCWHYAPKPCGIVSASKVCGPVYCFTPSPVVVGTTDRFGVPTYSWGENETDVLLLNNTRPPQGNWFGCTWMNGTGFTKTCGGPPCNIGGGGNNTLTCPTDCFRKHPAATYTKCGSGPWLTPRCLVDYPYRLWHYPCTANFTIFKVRMYVGGVEHRLDAACNWTRGERCNLEDRDRLELSPLLLSTTEWQVLPCSFTTLPALSTGLIHLHQNIVDVQYLYGIGSAVVSFAIKWDYIVILFLLLADARVCACLWMMLLIAQAEA

SEQ ID NO: 16 - protein, 747 aa, Hepatitis C virus
MSTNPKPQRKTKRNTNRRPQDVKFPGGGQIVGGVYLLPRRGPRLGVRAIRKTSERSQPRGRRQPIPKARRPEGRAWAQPGYPWPLYGNEGMGWAGWLLSPRGSRPSWGPTDPRRRSRNLGKVIDTLTCGLADLMGYIPLVGGPLGGAARALAHGVRVLEDGVNYATGNLPGCSFSIFLLALLSCLTVPASAHEVRNASGVYHVTNDCSNSSIVFEAADLIMHTPGCVPCVREGNSSRCWVALTPTLAARNATIPTTTIRHHVDLLVGAAALCSAMYVGDLCGSVFLVSQLFTFSPRRHATLQDCNCSIYPGHASGHRMAWDMMMNWSPTTALVVSQLLRIPQAVIDMVAGAHWGVLAGLAYYSMAGNWAKVLIVMLLFAGVDGHTLTTGGHAARLTSGFAGLFTPGPSQRIQLINTNGSWHINRTALNCNDSLQTGFLAALFYAHRFNSSGCPERMASCRSIDKFDQGWGPITYAEPTKDPDQRPYCWHYPPQQCGIVPASQVCGPVYCFTPSPVVVGTTDRLGNPTYSWGENDTDVLLLNNTRPPQGNWFGCTWMNSTGFTKTCGAPPCNIGGVGNNTLTCPTDCFRKHPEATYSKCGSGPWLTPRCMVDYPYRLWHYPCTVNFSIFKVRMYVGGVEHRLNAACNWTRGERCNLDDRDRSELSPLLLSTTEWQVLPCSFTTLPALSTGLIHLHQNIVDVQYLYGIGSAVVSFAIKWEYVVLLFLLLADARVCACLWMMLLIAQAEA

SEQ ID NO: 17 - protein, 746 aa, Hepatitis C virus
MSTNPKPQRKTKRNTNRRPQDVKFPGGGQIVGGVYLLPRRGPRLGVRATRKTSERSQPRGRRQPIPKARHPEGRTWAQPGYPWPLYGNEGLGWAGWLLSPRGSRPSWGPTDPRRRSRNLGKVIDTLTCGFADLMGYIPLVGAPLGGVARALAHGVRVLEDGVNYATGNLPGCSFSIFLLALLSCLTIPASAYEVRNVSGVYHVTNDCSNSSIVYEAADVIMHTPGCVPCVQDGNTSRCWVALTPTLAARNASVPVTAIRRHVDLLVGTAAFCSAMYVGDLCGSVFPVSQLFTFSPRRHQTVQDCNCSIYPGHISGHRMAWDMMMNWSPTAALVVSQLLRIPQAIVDMVAGAHWGVLAGLAYYSMVGNWAKVMIVLLLFAGVDGTTHTTGGAAARATQGFTSFFSLGPSQKIQLINTNGSWHINRTALNCNDSLQTGFLAALFYTYRFNASGCPERMASCRPIDKFDQGWGPITYAEPDSSDQRPYCWHYAPRPCGIVPASQVCGPVYCFTPSPVVVGTTDRFGVPTYTWGENETDVLLLNNTRPPLGNWFGCTWMNSTGFTKTCGGPPCNIGGAGNTTLTCPTDCFRKHPEATYTKCGSGPWLTPRCLVDYPYRLWHYPCAVNFTIFKVRMYVGGVEHRLNAACNWTRGERCDLEDRDRSELSPLLLSTTEWQVLPCSFTTLPALSTGLIHLHQNIVDVQYLYGIGSAVIPFAIKWEYVLLLFLLLADARVCACLWMMLLIAQAEA

SEQ ID NO: 18 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNISRCWVALTPTLAARNISVPTATIRRHVDLLVGTAAFCSAMYVGDLCGSVFLVSQLFTFSPRWHETVQDCNCSLYPGHVSGHRMAWDMMMNWSPTAALVVSQLLRIPQAVVDMVAGAHWGVLAGLAYYSMIGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNSTGYTKTCGGPPCNIGGVGNNTLTCPTDCFRKHPEATYTKCGSGPWLTPRCLVHYPYRLWHYPCTVNFTIFKVRMYVGGIEHRLDAACNWTRGERCDLEDRDRSELSPLLLSTTEWQILPCSFTTLPALSTGLIHLHRNIVDVQYLYGIGSAVVSFAIKWEYVLLLFLLLADARVCACLWMMLLIAQAEA

SEQ ID NO: 19 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGLGWAGWLLSPRGSRPSWGPNDPRRRSRNLGKVIDTLTCGFADLMGYIPLVGAPLGGAARALAHGVRVLEDGVNYATGNLPGCSFSIFLLALLSCLTIPASAYEVRNVSGIYHVTNDCSNSSIVYEAADVIMHAPGCVPCVRENNSSRCWVALTPTLAARNASVPTTTLRRHVDLLVGTAAFCSAMYVGDLCGSVFLISQLFTFSPRRHETVQDCNCSIYPGHVSGHRMAWDMMMNWSPTAALVVSQLLRIPQAVMDMVAGAHWGVLAGLAYYSMVGNWAKVLIVMLLFAGVDGHTRVTGGVQGHVTSTLTSLFRPGASQKIQLVNTNGSWHINRTALNCNDSLKTGFLAALFYTHKFNASGCPERMASCRSIDKFDQGWGPITYAQPDNSDQRPYCWHYAPRQCGIVPASQVCGPVYCFTPSPVVVGTTDRFGAPTYNWGDNETDVLLLNNTRPPHGNWFGCTWMNSTGFTKTCGGPPCNIRGVGNNTLTCPTDCFRKHPDATYTKCGSGPWLTPRCLVDYPYRLWHYPCTVNFTIFKVRMYVGGVEHRLDAACNWTRGERCDLEDRDRAELSPLLLSTTEWQILPCSYTTLPALSTGLIHLHQNIVDIQYLYGIGSAVVSIAIKWEYVVLLFLLLADARVCACLWMMLLIAQAEA

SEQ ID NO: 20 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGDEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNTSRCWVALTPTLAARNISVPTTTIRRHVDLLVGTAAFCSAMYVGDLCGSVFLVSQLFTFSPRRHETVQDCNCSLYPGHVSGHRMAWDMMMNWSPTAALVVSQLLRIPQAIVDMVAGAHWGVLAGLAYYSMVGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNNTGFTKTCGGPPCNIGGVGNNTLTCPTDCFRKHPEATYTKCGSGPWLTPRCLVDYPYRLWHYPCTVNFTVFKVRMYVGGIEHRLDAACNWTRGERCDLEDRDRSELSPLLLSTTEWQILPCSFTTLPALSTGLIHLHRNIVDVQYLYGIGSAVVSFAIKWEYILLLFLLLADARVCACLWMMLLIAQAEA

SEQ ID NO: 21 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVREDNSSRCWVALTPTLAARNSSVPTTTIRRHVDLLVGAAAFCSAMYVGDLCGSVFLISQLFTFSPRRYETVQDCNCSLYPGHVSGHRMAWDMMMNWSPTTALVVSQLLRIPQAVVDMVAGAHWGVLAGLAYYSMVGNWAKVLIVMLLFAGVDGNTRVSGGEAAKNTMGFASLFVSGPSQKIQLINTNGSWHINRTALNCDDSLHTGFLAALFYAHKFNSSGCSGRMASCRPIDEFAQGWGPITHGVPDNLDQRPYCWHYAPRPCGIVPASQVCGPVYCFTPSPVVVGTTDRFGAPTYSWGENETDVLLLNNTRPPQGNWFGCTWMNSTGFTKTCGGPPCNIGGVGNNTLTCPTDCFRKHPEATYTKCGSGPWLTPRCMVDYPYRLWHYPCTVNFTIFKVRMYVGGVEHRLDAACNWTRGERCNVEDRDRSELSPLLLSTTEWQILPCSFTTLPALSTGLIHLHQNIVDVQYLYGVGSVVVSVVIRWEYVVLLFLLLADARVCACLWMMLLIAQAEA

SEQ ID NO: 22 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGMYHVTNDCSNSSIVYEAADMILHAPGCVPCVRENNSSRCWVALTPTLAARNASVPTTAIRRHVDLLVGAAAFCSAMYVGDLCGSVLLVSQIFTFSPRRHETMQDCNCSIYPGHITGHRMAWDMMMNWSPTTALVVSQLLRIPQAIVDMVAGAHWGVLAGLAYYSMVGNWAKVLIVMLLFAGVDGGTYVTGGEAGRRTSGFASIFTPGASQNIQLINTNGSWHINRTALNCNDSLHTGFIAALFYHHKFNASGCPERMASCRPIGEFAQGWGPISYTEPPSSDQRPYCWHYPPRPCGIVPASQVCGPVYCFTPSPVVVGTTDRLGAPTYNWGDNDTDVLLLNNTRPPQGNWFGCTWMNGTGFTKTCGGPPCNIGGVGNNTLTCPTDCFRKHPEATYTKCGSGPWLTPRCIVDYPYRLWHYPCTVNFTITKIRMYVGGVEHRLTAACNWTRGERCDLEDRDRSELSPLLLSTTEWQIMPCSFTTLPALSTGLIHLHQNIVDIQYLYGIGSAAVSFAIRWEYVLLLFLLLADARVCACLWMMLLIAQAEA

SEQ ID NO: 23 - protein, 746 aa, Hepatitis C virus; identical to SEQ ID NO: 22

SEQ ID NO: 24 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGTARALAHGVRVLEDGVNYATGNLPGCSFSIFLLALLSCLTIPASAYEVRNVSGVYHVTNDCSNSSIVYETADMIMHTPGCVPCVREDNFTRCWVALTPTLAARNGSVPTTAIRRHVDLLVGAAAFCSAMYVGDLCGSVFLVSQLFTFSPRWHETVQECNCSIYPGHVTGHRMAWDMMMNWSPTTALVVSQLLRIPQAVVDMVAGAHWGVLAGLAYYSMVGNWAKVLIVTLLFAGVDGNTHTIGGKQAQATGGFVAWLARGPSQEIQLINTNGSWHINRTALNCNDSLKTGFIAALFYAHRFNSSGCPERMASCRPIDKFAQGWGPITYAKPDSLDQRPYCWHYAPQPCGIVPASEVCGPVYCFTPSPVVVGTTDRSGVPTYRWGENETDVLLLNNTRPPQGNWFGCTWMNATGFTKTCGGPPCKIGGLGNNTLTCPTDCFRKHPEATYTKCGSGPWLTPRCIVDYPYRLWHYPCTVNFTIFKVRMYVGGIEHRLSAACNWTRGERCDLEDRDRSELSPLLLSTTEWQILPCSFTTLPALSTGLIHLHQNTVDVQYLYGVGSVLVSFAIKWEYILLFFLLLADARVCACLWMMLLIAQAEA

SEQ ID NO: 25 - protein, 746 aa, Hepatitis C virus
MSTNPKPQRKTKRNTNRRPQNVKFPGGGQIVGGVCLLPRRGPRVGVRATRKTSERSQPRGRRQPIPKARRPEGRSWAQPGYPWPLYGNEGCGWAGWLLSPRGSRPSRGPSDPRRRSRNLGKVIDTLTCGFADLMGYIPLVGAPLGGAARALAHGVRVLEDGVNYATGNLPGCSFSIFLLALLSCLTVPASAVEVRNSSGIYHVTNDCPNASVVYETDSLIIHLPGCVPCVREGNASRCWVSLSPTVAAKDPGVPVNEIRRHVDLIVGAAAFCSAMYVGDLCGSIFLVGQLFTLSPRRHWTTQDCNCSIYPGHVTGHRMAWDMMMNWSPTGALVVAQLLRIPQAVLDMIAGAHWGVLAGPAYYSMVGNWAKVLVVLLLFAGVDATTQVTGGTAGRNAYRLASLFSTGPSQNIQLINSNGSWHINRTALNCNDSLHTGWVAALFYSHKFNSSGRPERMASCRPLTAFDQGWGPITYGGKASNDQRPYCWHYAPRPCGIVPAKEVCGPVYCFTPSPVVVGTTDKYGVPTYTWGENETDVLLLNNSRPPIGNWFGCTWMNSTGFTKTCGAPACNVGGSETNTLSCPTDCFRRHPDATYAKCGSGPWLNPRCMVDYPYRLWHYPCTVNYTIFKIRMFVGGIEHRLTAACNWTRGERCDLDDRDRAELSPLLLSTTQWQVLPCSFTTLPALSTGLIHLHQNIVDVQYLYGLSSVVTSWAIRWEYVVLLFLLLADARICACLWMMLLISQVEA

SEQ ID NO: 26 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTYGFADLMGYIPLVGAPLGGAARALAHGVRVLEDGVNYATGNLPGCSFSIFLLALLSCLTVPASAVGVRNSSGVYHVTNDCPNASVVYETDSLIIHLPGCVPCVREGNGSRCWVSLSPTVAAKDPGVPVNEIRRHVDLIAGAAAFCSAMYVGHLCGSIFLVGQLFTLSPRRHWTTQDCNCSIYPGHVTGHRMAWDMMMNWSPTTALVVAQLLRIPQAILDMIAGAHWGVLAGIAYFSMVGNWTKVLVVLLLFAGVDATTIVSGGSAGRSTAGLVGLFSPGARQNIQLINTNGSWHINRTALNCNDTLQTGWVAGLFYTNKFNSSGCPERLASCRPLADFDQGWGPISYTNGSGPDQRPYCWHYPPKPCGIVPAESVCGPVYCFTPSPVVVGTTDRSGAPTYNWGENETDVFVLNNTRPRLGNWFGGTWMNSTGFTKVCGAPPCAIGGVGNNTLYCPTDCFRKHPEATYSRCGSGPWITPRCLIHYPYRLWHYPCTINYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 27 - protein, 746 aa, Hepatitis C virus
MSTNPKPQRKTKRNTNRRPQDVKFPGGGQIVGGVYLLPRRGPRVGVRATRKTSERSQPRGRRQPIPKARRPEGRSWAQPGYPWPLYGNEGCGWAGWLLSPRGSRPSWGPSDPRRRSRNLGKVIDTLTCGFADLMGYIPLVGAPLGGAARALAHGVRVLEDGVNYATGNLPGCSFSIFLLALLSCLTVPASAVGVRNSSGVYHVTNDCPNASVVYETENLIMHLPGCVPYVREGNASRCWVSLSPTVAARDSRVPVSEVRRRVDSIVGAAAFCSAMYVGDLCGSIFLVGQIFTFSPRHHWTTQDCNCSIYPGHVTGHRMAWDMMMNWSPTGALVVAQLLRIPQAIVDMIAGAHWGVLAGLAYYSMVGNWAKVVVVLLLFAGVDAETRVTGGAAGHTAFGFASFLAPGAKQKIQLINTNGSWHINRTALNCNESLDTGWLAGLLYYHKFNSSGCPERMASCQPLTAFDQGWGPITHEGNASDDQRPYCWHYALRPCGIVPAKKVCGPVYCFTPSPVVVGTTDRAGVPTYRWGANETDVLLLNNSRPPMGNWFGCTWMNSSGFTKTCGAPACNIGGSGNNTLLCPTDCFRKHPDATYSRCGSGPWLTPRCLVDYPYRLWHYPCTVNYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 28 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIMAGAHWGILAGLAYYSMQGNWAKVAIIMVMFSGVDAHTYTTGGTASRHTQAFAGLFDIGPQQKLQLVNTNGSWHINSTALNCNESINTGFIAGLFYYHKFNSTGCPQRLSSCKPITFFRQGWGPLTDANITGPSDDRPYCWHYAPRPCDIVPASSVCGPVYCFTPSPVVVGTTDARGVPTYTWGENEKDVFLLKSQRPPSGRWFGCSWMNSTGFLKTCGAPPCNIYGGEGNPHNESDLFCPTDCFRKHPETTYSRCGAGPWLTPRCMVDYPYRLWHYPCTVDFRLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQTEA

SEQ ID NO: 29 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGQNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVSHVLRLPQTLFDIIAGAHWGILAGLAYYSMQGNWAKVAVIMVMFSGVDAETYITGGSAAHGVSTLTSLFSSGPQQKLQLVKTNGSWHINSTALNCNESINTGFIAGLFYYHKFNSTGCPQRLSSCKPITFFRQGWGSLTDANVTGASADKPYCWHYAPRPCDVVPALNVCGPVYCFTPSPVVVGTTDRKGVPTYNWGENESDVFLLESLRPPSGRWFGCAWMNSTGFLKTCGAPPCNIYGGGGNPNNESHLFCPTDCFRKHPDATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGMVGWALKWEFVILIFLLLADARVCVALWLMLMISQAEA

SEQ ID NO: 30 - protein, 752 aa, Hepatitis C virus
MSTLPKPQRKTKRNTIRRPQDVKFPGGGQIVGGVYVLPRRGPRLGVCATRKTSERSQPRRRRQPIPKARQSGGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQNDNISTCWTPVTPTVAVRYVGATTASIRSHVDLLVGAATMCSALYVGDMCGAVFLVGQAFTFRPRRHQTVQTCNCSLYPGHLSGHRMAWDMMMNWFPALGMAVAHVLRVPQTLFDIIAGAHWGILAGLAYYSMQGNWAKVAIIMVMFSGVDAVTYTTGGSAAHATRGLTSLFSVGAQQKLQLVNTNGSWHINSTALNCNESINTGFIAGLFYYHRFNSTGCPQRLSSCKPITFFKQGWGPLTDANISGPSDDKPYCWHYAPRPCKVVPASGVCGPVYCFTPSPVVVGTTDAKGVPTYTWGANDTDVFLLESLRPPGGRWFGCTWMNSTGFVKTCGASPCDIYGGGGNSGNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILIFLLLADRRVCVALWLMLMITQAEA

SEQ ID NO: 31 - protein, 752 aa, Hepatitis C virus
MSTLPKPQRKTKRNTVCRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNVSGLYILTNDCPNSSIVYEADDVILHTPGCIPCVQDGNTSTCWTALTPTVAVRYVGATTASIRSHVDLLVGAATMCSALYVGDMCGAVFLVGQAFTFRPRRHQTVQTCNCSLYPGHLTGHRMAWDMMMNWSPAVGMVVAHVLRLPQTLFDIIAGAHWGILAGLAYYSMQGNWAKVIIIMVMFSGVDATTHVTGGTAGLTAFRLTGLFTVGPQQKLQLVNTNGSWHINRTALNCNDSLNTGFIAGLFRFHKFNSTGCPEMLSSCKPITSFKQGWGPLTDANITIPSDDRPYCWHYPPRSCEVVPALSVCGPVYCFTPSPVVVGTTDAKGVPTYTWGENETDVFLLKSLRPPGGRWFGCTWMNSTGFVQTCGAPPCNIYGGGGDLKNESDLFCPTDCFRKHPEATYSRCGAGPWLTPRCMVDYPYRLWHYPCTVNFTLFKVRMFVGGFEHRFTAACNWTRGERCDIEDRDRSELHPLLHSTTELAILPCSFTPMPALSTGLIHLHQNIVDVQYLYGVGSGMVGWAVKWEFVILVFLLLADARVCVALWLMLMISQAEA

SEQ ID NO: 32 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLQWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAAGMVVAHILRLPQTLFDIIAGAHWGILAGLAYYSMQGNWAKVAIIMVMFSGVDATTYTSGGSVAQQARGLADLFSVGAKQNLQLVNTNGSWHINSTALNCDDSINTGFIAGLFYYHKFNSTGCPQRLSDCKPITFFKQGWGPLTDANITGPSDDKPYCWHYAPRRCGVVPASSVCGPVYCFTPSPVVVGTTDAKGVPTYTWGANETDVFLLESLRPPSGRWFGCAWMNSTGFLKTCGAPPCNIYGGGGNPHNESDLFCPTDCFRKHPEATYSRCGAGPWLTPRCMVDYPYRLWHYPCTVNFTLFKMRTFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQAEA

SEQ ID NO: 33 - protein, 752 aa, Hepatitis C virus; identical to SEQ ID NO: 32

SEQ ID NO: 34 - protein, 752 aa, Hepatitis C virus
MSTLPKPQRKTKRNTIRRPQDVKFPGGGQIVGGVYVLPRRGPRLGVCATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLVHPAASLEWRNTSGLYVLTNDCPNSSIVYEADDVILHTPGCIPCVQDGNKSTCWTSVTPTVAVKYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGNAARGASGIVSLFTPGAKQNLQLVNTNGSWHINRTALNCNDSINTGFIAGLIYYHKFNSTGCPQRLSSCKPITFFRQGWGSLTDANITGPSDDKPYCWHYPPRPCDTIRASSVCGPVYCFTPSPVVVGTTDAKGAPTYNWGANETDMFLLQSLRPPSGRWFGCTWMNSTGFTKTCGAPPCNIYGGGGNLNNESDLFCPTDCFRKHPEATYSRCGAGPWLTPRCLVDYPYRLWHYPCTVNFTLFRMRTFVGGFEHRFTAACNWTRGERCNIEDRDRSEQHPLLHSTTELAILPCSFTPMPALSTGLIHLHQNIVDVQYLYGIGSGVVGWALKWEFVILVFLLLADARVCVALWLMLMISQAEA

SEQ ID NO: 35 - protein, 752 aa, Hepatitis C virus
MSTLPKPQRKTKRNTIRRPQDVKFPGGGVIYVGVYVLPRRGPRLGVRATRKTSERSQPRGRRKPIPKARRSEGRSWAQPGYPWPLYGNEGCGWAGWLLSPRGSRPNWAPNDPRRRSRNLGKVIDTLTCGFADLMGYIPLVGAPLGGAARALAHGVRALEDGINFATGNLPGCSFSIFLLALFSCLIHPAASLEWRNTSGLYVLTNDCSNSSIVYEADDVILHTPGCIPCVQDGNTSTCWTPVTPTVAVRYVGATTASIRSHVDLLVGAGTMCSALYVGDMCGPVFLVGQAFTFRPRRHRTVQTCNCSLYPGHLSGQRMAWDMMMNWSPAVGMVVAHILRLPQTLFDVVAGAHWGIIAGLAYYSMQGNWAKVAIIMVMFSGVDASTHVTAGQAARNAYGITSLFSVGAKQNLQLINTNGSWHINRTALNCNESINTGFIAGLFYYHKFNSTGCPQRLSSCKPITFFKQGWGPLTDANITGPSDDKPYCWHYAPRPCGIVPALNVCGPVYCFTPSPVVVGTTDAKGAPTYTWGANKTDVFLLESLRPPSGRWFGCTWMNSTGFVKTCGAPPCNIYGDGRDAQNESDLFCPTDCFRKHPEATYSRCGAGPWLTPRCLVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGIGSGMVGWALKWEFVILIFLLLADARVCVALWLILTISQAEA

SEQ ID NO: 36 - protein, 754 aa, Hepatitis C virus
MSTLPKPKRQTKRNTLRRPKNVKFPAGGQIVGEVYVLPRRGPQLGVREVRKTSERSQPRGRRQPTPKARPREGRSWAQPGYPWPLYGNEGCGWAGWLLPPRGSRPSWGQNDPRRRSRNLGKVIDTLTCGFADLMGYIPLIGAPVGGVARALAHGVRALEDGVNYATGNLPGCSFSIFLLALFSCLTCPASSLEYRNASGLYLLTNDCSNRSIVYEADDVILHLPGCVPCVETDNNNTSCWTPISPTVAVKHPGVTTASIRNHVNMLVAPPTLCSALYVEDAFGAVSLVGQAFTFRPRQHKTVQTCNCSIYPGHVSGHRMAWDMMMNWSPAIGLVISHLMRLPQTFFDLVVGAHWGVMAGLAYFSMQGNWAKVVIVLIMFSGVDATTHTTGGSAAQATAGFTSFFTRGPSQNLQLVNSNGSWHINSTALNCNDSLNTGFIAGLFYYHKFNSSGCPERMSSCKPITYFNQGWGPLTDANINGPSEDRPYCWHYPPRPCNITKPLNVCGPVYCFTPSPVVVGTTDIKGLPTYRFGVNESDVFLLTSLRPPQGRWFGCVWMNSTGFVKTCGAPPCNIYGGMKDIEANQTHLKCPTDCFRKHHDATFTRCGSGPWLTPRCLVDYPYRLWHYPCTVNFSIFKVRMFVGGHEHRFSAACNWTRGERCDLEDRDRSEQQPLLHSTTDSLILPCSFTPMRRLSTGLIHLHQNIVDVQYLYGVGSAVVGWALKWEFVVLVFLLLADARVCVALWMMLLISQAEA

SEQ ID NO: 37 - protein, 751 aa, Hepatitis C virus
MSTLPKPQRITKRNINRRPQDVKFPGGGQIVGGVYVLPRRGPKLGVRAVRKTSERSQPRSRRQPIPRARRTEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALLSCLLTPTAGLEYRNASGLYTVTNDCSNGSIVYEAGDVILHLPGCIPCVRLNNASKCWTPVSPTVAVSRPGAATASLRTHVDMMVGAATLCSALYVGDLCGALFLVGQGFSWRHRQHWTVQDCNCSIYPGHLTGHRMAWDMMMNWSPAMTLIVSQVLRLPQTMFDLVIGAHWGVMAGVAYYSMQGNWAKVFLVLCLFSGVDASTTITGGVAASGAFTITSLFSTGAKQPLHLVNTNGSWHINRTALNCNDSLNTGFIAGLLYYHKFNSSGCVERMSACSPLDRFAQGWGPLGPANISGPSSEKPYCWHYAPRPCDTVPAQSVCGPVYCFTPSPVVVGATDKRGAPTYTWGENESDVFLLESARPPTEPWFGCTWMNGSGYVKTCGAPPCHIYGGREGKSNNSLVCPTDCFRKHPDATYNRCGAGPWLTPRCLVDYPYRLWHYPCTVNYTIFKVRMFVGGLEHRFNAACNWTRGERCNLEDRDRSEMYPLLHSTTEQAILPCSFVPIPALSTGLIHLHQNIVDVQYLYGISSGLVGWAIKWEFVILIFLLLADARVCVVLWMMMLISQAEA

SEQ ID NO: 38 - protein, 745 aa, Hepatitis C virus
  misc_feature (198)..(198): Xaa can be any naturally occurring amino acid
  misc_feature (217)..(217): Xaa can be any naturally occurring amino acid
  misc_feature (223)..(223): Xaa can be any naturally occurring amino acid
  misc_feature (235)..(235): Xaa can be any naturally occurring amino acid
  misc_feature (242)..(242): Xaa can be any naturally occurring amino acid
  misc_feature (268)..(268): Xaa can be any naturally occurring amino acid
  misc_feature (297)..(297): Xaa can be any naturally occurring amino acid
  misc_feature (313)..(313): Xaa can be any naturally occurring amino acid
  misc_feature (329)..(329): Xaa can be any naturally occurring amino acid
  misc_feature (344)..(344): Xaa can be any naturally occurring amino acid
  misc_feature (379)..(379): Xaa can be any naturally occurring amino acid
  misc_feature (390)..(390): Xaa can be any naturally occurring amino acid
  misc_feature (392)..(392): Xaa can be any naturally occurring amino acid
  misc_feature (394)..(394): Xaa can be any naturally occurring amino acid
  misc_feature (396)..(396): Xaa can be any naturally occurring amino acid
  misc_feature (402)..(404): Xaa can be any naturally occurring amino acid
  misc_feature (406)..(406): Xaa can be any naturally occurring amino acid
  misc_feature (411)..(411): Xaa can be any naturally occurring amino acid
  misc_feature (423)..(423): Xaa can be any naturally occurring amino acid
  misc_feature (430)..(430): Xaa can be any naturally occurring amino acid
  misc_feature (441)..(441): Xaa can be any naturally occurring amino acid
  misc_feature (443)..(445): Xaa can be any naturally occurring amino acid
  misc_feature (473)..(474): Xaa can be any naturally occurring amino acid
  misc_feature (479)..(479): Xaa can be any naturally occurring amino acid
  misc_feature (527)..(527): Xaa can be any naturally occurring amino acid
  misc_feature (530)..(530): Xaa can be any naturally occurring amino acid
  misc_feature (569)..(569): Xaa can be any naturally occurring amino acid
  misc_feature (579)..(579): Xaa can be any naturally occurring amino acid
  misc_feature (590)..(590): Xaa can be any naturally occurring amino acid
  misc_feature (607)..(607): Xaa can be any naturally occurring amino acid
  misc_feature (625)..(625): Xaa can be any naturally occurring amino acid
  misc_feature (709)..(709): Xaa can be any naturally occurring amino acid
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSXGLYHVTNDCPNSSIVYEAXDAILHXPGCVPCVREGNXSRCWVAXTPTVATRDGKLPTTQLRRHIDLLVGXATLCSALYVGDLCGSVFLVGQLFTFSPRXHWTTQDCNCSYPGHIXGHRMAWDMMMNWSPTXALVVAQLLRIPQAIXDMIAGAHWGVLAGIAYFSMVGNWAKVLVVLLLFAXVDAETHVTGGXAXRXTXGLVGLXXXGXKQBIXLINTNGSWHINXTALNCNXSLNTGWLAGLXYXXXFNSSGCPERLASCRRLTDFAQGWGPISXXNGSGXDERPYCWHYPPRPCGIVPAKSVCGPVYCFTPSPVVVGTTDRSGAPTYXWGXNDTDVFVLNNTRPPLGNWFGCTWMNSTGFTKVCGAPPCXIGGVGNNTLXCPTDCFRKHPXATYSRCGSGPWITPRCXVDYPYRLWHYPCTINYTXFKVRMYVGGVEHRLEAACNWTRGERCBLEDRDRSELSPLLLSTTQWQVLPCSFTTLPALSTGLIHLHQNIVDVQYLYGVGSSIXSWAIKWEYVVLLFLLLADARVCSCLWMMLLISQAEA

SEQ ID NO: 39 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEADDAILHAPGCVPCVREGNISRCWVAMTPTVATRDGKLPTTQLRRHIDLLVGGATLCSALYVGDLCGSVFLVGQLFTFSPRLHWTTQDCNCSLYPGHISGHRMAWDMMMNWSPTTALVVAQLLRIPQAILDMIAGAHWGVLAGIAYFSMVGNWAKVLVVLLLFASVDAETHVTGGNAARSTSGLVGLFSAGPKQDIRLINTNGSWHINRTALNCNDSLNTGWLAGLLYRYNFNSSGCPERLASCRRLTDFAQGWGPISHVNGSGPDERPYCWHYPPRPCGIVPAKSVCGPVYCFTPSPVVVGTTDRSGAPTYNWGENDTDVFVLNNTRPPLGNWFGCTWMNSTGFTKVCGAPPCAIGGVGNNTLRCPTDCFRKHPDATYSRCGSGPWITPRCLVDYPYRLWHYPCTINYTVFKVRMYVGGVEHRLEAACNWTRGERCNLEDRDRSELSPLLLSTTQWQVLPCSFTTLPALSTGLIHLHQNIVDVQYLYGVGSSIVSWAIKWEYVVLLFLLLADARVCSCLWMMLLISQAEA

SEQ ID NO: 40 - protein, 750 aa, Hepatitis C virus
MSTNPKPQRLTKRNTVRRPQNVKFPGGGQIVGGVYLLPRRGPRLGVRGTRKSSERSQPRGRRQRIPKAASSQGKAWGKPGYPWPLYGNEGCGWAGWLLSPRGSRPTWGPTDPRHRSRNLGKVIDTMTCGFADLMGYIPVLGAPLGGVARALAHGVRVLEDGVNYATGNLPGCSFSIFLLALLSCLTVPASAYEVRNSSGVYHLTNDCPNASIVYETDNAILHEPGCVPCVREGNTSRCWEPVAPTLAVRYRGALTDDLRTHIDLVVASATLCSALYVGDICGAIFIASQAVLWKPGGGRIVQDCNCSIYPGHVTGHRMAWDMMQNWAPALSMVAAYAVRVPGVIITTVAGGHWGVLFGLAYFGMAGNWAKVILIMLLMSGVDAETMAVGARAAHTTGALVSLLNPGPSQRLQLINTNGSWHINRTALNCNDSLQTGFIAALFYTHRFNSSGCPERMASCKPLSDFDQGWGPLWYNSTERPSDQRPYCWHYAPSPCGIVPAKDVCGPVYCFTPSPVVVGTTDRRGVPTYTWGENESDVFLLNSTRPPQGSWFGCSWMNTTGFTKTCGGPPCKIRPQGAQSNTSLTCPTDCFRKHPRATYSACGSGPWLTPRCMVHYPYRLWHYPCTVNFTIHKVRLYIGGVEHRLDAACNWTRGERCDLEDRDRVDMSPLLHSTTELAILPCSFVPLPALSTGLIHLHQNIVDAQYLYGLSPAIISWAIRWEWVVLVFLLLADARICACLWMMMLMAQAEA

SEQ ID NO: 41 - protein, 750 aa, Hepatitis C virus
MSTNPKPQRKTKRNTNRRPEDVKFPGGGQIVGGVYLLPRRGPRLGVRTTRKTSERSQPRGRRQPIPKDRRSTGKAWGKPGRPWPLYGNEGLGWAGWLLSPRGSRPSWGPTDPRHRSRNVGKVIDTLTCGFADLMGYIPVVGAPLSGAARAVAHGVRVLEDGVNYATGNLPGFPFSIFLLALLSCITVPVSAAQVKNTSSSYMVTNDCSNDSITWQLEAAVLHVPGCVPCERVGNTSRCWVPVSPNMAVRQPGALTQGLRTHIDMVVMSATFCSALYVGDLCGGVMLAAQVFIVSPQYHWFVQECNCSIYPGTITGHRMAWDMMMNWSPTATMILAYVMRVPEVIIDIVSGAHWGVMFGLAYFSMQGAWAKVIVILLLAAGVDAGTTTVGGAVARSTNVIAGVFSHGPQQNIQLINTNGSWHINRTALNCNDSLNTGFLAALFYTNRFNSSGCPGRLSACRNIEAFRIGWGTLQYEDNVTNPEDMRPYCWHYPPKPCGVVPARSVCGPVYCFTPSPVVVGTTDRRGVPTYTWGENETDVFLLNSTRPPQGSWFGCTWMNSTGFTKTCGAPPCRTRADFNASTDLLCPTDCFRKHPDATYIKCGSGPWLTPKCLVHYPYRLWHYPCTVNFTIFKIRMYVGGVEHRLTAACNFTRGDRCDLEDRDRSQLSPLLHSTTEWAILPCTYSDLPALSTGLLHLHQNIVDVQYMYGLSPAITKYVVRWEWVVLLFLLLADARVCACLWMLILLGQAEA

SEQ ID NO: 42 - protein, 749 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITTPVSAAQVKNTSDIYMVTNDCSNDSITWQLQAAALHVPGCVPCERVGNTSRCWIPVSPNVAVRQPGALTQGLRTHIDMVVMSATLCSALYVGDLCGGVMLAAQMFIISPQHHWFVQECNCSIYPGTITGHRMAWDMMMNWSPTTTMILAYVMRVPEVIIDIIGGAHWGVMFGLAYFSMQGAWAKVVVILLLAAGVDAHTRTGSSVGYATSGIVGLFTSGPKQNIQLINTNGSWHINRTALNCNDSLNTGFIVSLFYARNFNSTGCPERLSACRGIEGFRIGWGTLQYEDNVTNPEDMRPYCWHYPPKQCGIVPAGSVCGPVYCFTPSPVVVGTTDRLGVPTYTWGENETDVFLLNSTRPPVGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFTRGDRCNLEDRDRSQLTPLLHSTTEWAILPCTYSDLPALSTGLLHLHQNIVDVQYMYGLSPALTKYVVRWEWVVLLFLLLADARVCACVWMLILLGQAEA

SEQ ID NO: 43 - protein, 749 aa, Hepatitis C virus
  misc_feature (386)..(386): Xaa can be any naturally occurring amino acid
  misc_feature (392)..(394): Xaa can be any naturally occurring amino acid
  misc_feature (398)..(399): Xaa can be any naturally occurring amino acid
  misc_feature (444)..(444): Xaa can be any naturally occurring amino acid
  misc_feature (496)..(496): Xaa can be any naturally occurring amino acid
  misc_feature (525)..(525): Xaa can be any naturally occurring amino acid
  misc_feature (538)..(538): Xaa can be any naturally occurring amino acid
  misc_feature (712)..(712): Xaa can be any naturally occurring amino acid
  misc_feature (722)..(722): Xaa can be any naturally occurring amino acid
MSTNPKPQRKTKRNTNRRPQDVKFPGGGQIVGGVYLLPRRGPRLGVRATRKTSERSQPRGRRQPIPKHRRSTGKSWGTPGYPWPLYGNEGLGWAGWLLSPRGSRPTWGPTDPRHRSRNVGKVIDTLTCGFADLMGYIPVVGAPLGGVARALAHGVRVLEDGVNYATGNLPGCSFSIFLLALLSCITIPVSAAQVKNTSSGYMVTNDCSNDSITWQLEAAVLHVPGCVPCEKVGNTSRCWIPVSPNVAVQRPGALTQGLRTHIDMVVMSATLCSALYVGDFCGGVMLAAQMFIVSPQHHWFVQECNCSIYPGTISGHRMAWDMMMNWSPTATMIMAYVMRVPEVIIDIIAGAHWGVMFGLAYFSMQGAWAKVVVILLLTAGVDAHTXIGSAVXXXTSGXXGLFYRGAQQNIQLINTNGSWHINRTALNCNDSLNTGFLASLFYAXRFNSSGCPERLSVCRNIEAFRIGWGTLQYEDNVTNPEDMRPYCWHYPPKPCXIVPAGSVCGPVYCFTPSPVVVGTTDRLGXPTYTWGENETDVXLLNSTRPPQGSWFGCTWMNSTGFTKTCGAPPCRTRADFNASTDLLCPTDCFRKHPEATYIKCGSGPWLTPRCMVDYPYRLWHYPCTVNYSIFKIRMYVGGVEHRLMAACNFTRGDRCDLEDRDRSQLSPLLHSTTEWAILPCSYSDLPALSTGLLHLHQNIVDVQYMYGLSPAXTKYVVRWEWXVLLFLLLADARVCACLWMLILLGQAEA

SEQ ID NO: 44 - protein, 750 aa, Hepatitis C virus
MSTNPKPQRKTKRNTSRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITVPVSAVQVKNISDSYMVTNDCSNDSITWQLQAAVLHVPGCVPCEKMGNISRCWIPVSPNVAVQRPGALTQGLRAHIDMVVMSATLCSALYVGDLCGGVMLAAQMFIVSPQHHHFVQECNCSIYPGAITGHRMAWDMMMNWSPTATMILAYAMRVPEVIIDIISGAHWGVMFGLAYFSMQGAWAKVVVILLLTAGVDAHTRSIAGSVAHATSGLAGLFTSGAKQNIQLINTNGSWHINRTALNCNDSLNTGFIASLFYTYRFNSSGCPERLSACRGIQAFRIGWGTLRYEDNVTNPEDMRPYCWHYPPKQCGIVSARSVCGPVYCFTPSPVVVGTTDRLGVPTYTWGENETDVFILNSTRPPGGSWFGCTWMNSTGFTKTCGAPPCRIRADFNASMDLLCPTDCFRKHPDATYIKCGSGPWLTPRCLVDYPYRLWHYPCTINYTIFKIRMYVGGVEHRLTAACNFTRGDPCNLEDRDRSQLSPLLHSTTEWAILPCSYSDLPALSTGLLHLHQNIVDVQYMYGLSPALTKYIVRWEWVVLLFLLLADARVCACLWMLILLGQAEA

SEQ ID NO: 45 - protein, 750 aa, Hepatitis C virus
  misc_feature (458)..(458): Xaa can be any naturally occurring amino acid
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCVTVPVSAVQVKNTSETYMVTNDCSNDSITWQLQAAVLHVPGCVPCERTGNTSRCWIPVSPNVAVQQPGALTQGLRTHIDMVVMSATLCSALYVGDLCGGVVLAAQLFIVSPRRHWFVQECNCSIYPGAITGHRMAWDMMMNWSPTTTMVLAYAMRVPEVIIDIISGAHWGVMFGLAYFSMQGAWAKVAVILLLTAGVEARTHTTGSVAGRTTSGFAGIFTSGPKQNIQLINTNGSWHINRTALNCNDSLNTGFMAALFYTKNFNSSGCPERLSXCRNIEAFRIGWGTLQYEDDVTNPEDMRPYCWHYPPKQCGIFPAGSVCGPVYCFTPSPVVVGTTNKLGVPTYTWGENETDVFILNSTRPPRGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFSRGDRCNLEDRDRSQLTPLLHSTTEWAILPCSYSDLPALSTGLLHLHQNIVDVQYMYGLTPALTKYVVRWEWVVLLFLLLADARVCACLWMLILLGQAEA

SEQ ID NO: 46 - protein, 751 aa, Hepatitis C virus
MSTNPKPQRKTQRNTNRRPQDVKFPGGGQIVGGVYLLPRRGPRLGVRTARKTSERSQPRGRRQPIPKDRRSTGKSWGRPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITIPASAVEVKNTSTGYMVTNDCANSSITWQLHAAVLHVPGCVPCERVDNNTSRCWIPVSPNIAVQRPGALTQGLRSHIDIVVMSATLCSALYVGDLCGGVMLAAQMFVVSPEHHWFVQECNCSIYPGTITGHRMAWDMMMNWSPTATMILAYAMRVPEVIIDIIGGAHWGVMFGLAYFSMQGAWAKVVVILLLAAGVDAYTHTVGGAAASTANSIAGLLSRGPRQNLQLINSNGSWHINRTALNCHDSLQTGFITALFYARHFNSSGCPERLAACRNIEAFRVGWGALQYEDNVTNPEDMRPYCWHYPPKQCGIVPARSVCGPVYCFTPSPVVVGTTDKLGVPTYTWGENETDVFLLNSTRPPQGPWFGCTWMNSTGFTKTCGAPPCRTRADFNASTDLLCPTDCFRKHPDATYNKCGSGPWLTPRCLIDYPYRLWHYPCTVNYTTFKIRMYVGGVEHRLMAACNFTRGDSCDLSQRDRGQLSPLLHSTTEWAILPCSFSDLPALSTGLLHLHQNIVDVQYMYGLSPALTKYIVRWEWVVLLFLLLADARVCACIWMLILLGQAEA

SEQ ID NO: 47 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNTSRCWIPVSPNVAVQQPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIIGGAHWGVMFGLAYFSMQGAWAKVVVILLLAAGVDAQTHTVGGSTAHNARTLTGMFSLGARQKIQLINTNGSWHINRTALNCNDSLHTGFLASLFYTHSFNSSGCPERMSACRSIEAFRVGWGALQYEDNVTNPEDMRPYCWHYPPRQCGVVSASSVCGPVYCFTPSPVVVGTTDRLGAPTYTWGENETDVFLLNSTRPPQGSWFGCTWMNSTGYTKTCGAPPCRIRADFNASMDLLCPTDCFRKHPDTTYIKCGSGPWLTPRCLIDYPYRLWHYPCTVNYTIFKIRMYVGGVEHRLTAACNFTRGDRCNLEDRDRSQLSPLLHSTTEWAILPCTYSDLPALSTGLLHLHQNIVDVQFMYGLSPALTKYIVRWEWVVLLFLLLADARVCACLWMLILLGQAEA

SEQ ID NO: 48 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDARTHTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEA

SEQ ID NO: 49 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCVTVPVSAVEVRNISSSYYATNDCSNNSITWQLTDAVLHLPGCVPCENDNGTLHCWIQVTPNVAVKHRGALTRSLRTHVDMIVMAATACSALYVGDVCGAVMILSQAFMVSPQRHNFTQECNCSIYQGHITGHRMAWDMMLSWSPTLTMILAYAARVPELVLEIIFGGHWGVVFGLAYFSMQGAWAKVIAILLLVAGVDATTYSSGQEAGRTVAGFAGLFTTGAKQNLYLINTNGSWHINRTALNCNDSLQTGFLASLFYTHKFNSSGCPERLSSCRGLDDFRIGWGTLEYETNVTNDGDMRPYCWHYPPRPCGIVPARTVCGPVYCFTPSPVVVGTTDKQGVPTYTWGENETDVFLLNSTRPPRGAWFGCTWMNGTGFTKTCGAPPCRIRKDYNSTIDLLCPTDCFRKHPDATYLKCGAGPWLTPRCLVDYPYRLWHYPCTVNFTIFKARMYVGGVEHRFSAACNFTRGDRCRLEDRDRGQQSPLLHSTTEWAVLPCSFSDLPALSTGLLHLHQNIVDVQYLYGLSPALTRYIVKWEWVILLFLLLADARICACLWMLIILGQAEA

SEQ ID NO: 50 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPSDPRHRSRNLGRVIDTITCGFADLMGYIPVVGAPVGGVARALAHGVRVLEDGINYATRNLPGCSFSIFLLALLSCVTVPVSSVEIRNISTSYYATNDCSNNSITWQLTNAVLHLPGCVPCENDNGTLRCWIQVTPNVAVKHRGALTHNLRAHVDVIVMAATVCSALYVGDVCGAVMIVSQALIVSPERHNFTQECNCSIYQGHITGQRMAWDMMLNWSPTLTMILAYAARVPELVLEIVFGGHWGVVFGLAYFSMQGAWAKVIAILLLVAGVDATTYSTGATVGRTVGSFAGLFKLGAQQNVQLINTNGSWHINRTALNCNDSLHTGFMAALFYANKFNSSGCPERLSSCRGLDDFRIGWGTLEYETNVTNVEDMRPYCWHYPPKPCGIVPAQSVCGPVYCFTPSPVVVGTTDRQGVPTYNWGDNETDVFLLNSTRPPRGAWFGCTWMNGTGFTKTCGAPPCRIRKDFNSTLDLLCPTDCFRKHPDATYVKCGAGPWLTPRCLIDYPYRLWHYPCTVNFTIFKVRMYVGGVEHRFSAACNFTRGDRCRLEDRDRGQQSPLLHSTTEWAVLPCSFSDLPALSTGLLHLHQNIVDVQYLYGLSPAVTKYIVKWEWVVLLFLLLADARICACLWMLIILGQAEA

SEQ ID NO: 51 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPHWGPTDPRHRSRNLGKVIDTITCGFADLMGYIPVIGAPVGGVARALAHGVRVLEDGVNYATGNLPGCSFSIFLLALLSCVTVPVSAVEVRNISSSYYATNDCSNNSITWQLENAVLHLPGCVPCENDNGTLRCWTQVTPNVAVKHRGALTQNLRTHVDVIVVAATVCSALYVGDVCGAVMIASQALIVSPARHNFTQECNCSIYQGRITGHHMAWDMMLNWSPTITMILAYAARIPELVLEVIFGGHWGVMFGLAYFSMQGAWAKVIVILLLVAGVDARHHTTGLQAGKTLARVTSLFSIGAKQNIQLINTNGSWHINRTALNCNDSLQTGFIASLFYVNNINSSGCPERMSSCRELDDFRIGWGTLEYETNVTNDEDMRPYCWHYPPKPCGIVPARTVCGPVYCFTPSPIVVGTTDKQGVPTYSWGENETDVFLLNSTRPPRGSWFGCTWMNGTGFTKTCGAPPCRIRRDYNSTLDLLCPTDCFRKHPDTTYLKCGSGPWLTPKCLVEYPYRLWHYPCTVNFTIFKVRMYVGAVEHRFSAACNFTRGDRCRLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 52 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCCTVPVSAVEVRNISTSYYATNDCSNTSITWQLTNAVLHLPGCVPCENDNGTLRCWIQVTPNVAVKHRGALTHNLRTHVDVIVMAATVCSALYVGDICGAVMIVSQAFIISPERHNFTQECNCSMYQGHITGHRMAWDMMLNWSPTLTMILAYAARVPELVLEVIFGGHWGVVFGLAYFSMQGAWAKVIAILLLVAGVDANTYSSGVTVGHTTSTFANIFSVGPSQKINLINTNGSWHINRTALNCNDSLQTGFLASLFYVRNFNSSGCRERLSSCRRLDDFRIGWGTLEYETNVTNDEDMRPYCWHYPPKPCGIVSARTVCGPVYCFTPSPVVVGTTDRQGVPTYSWGENETDVFLLNSTRPPRGAWFGCTWMNGTGFTKTCGAPPCRIRRDYNSTLDLLCPTDCFRKHPDTTYLKCGAGPWLTPKCLVDYPYRLWHYPCTVNFTIFKVRMYVGGVEHRLSAACNFTRGDRCGLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 53 - protein, 750 aa, Hepatitis C virus
  misc_feature (203)..(203): Xaa can be any naturally occurring amino acid
  misc_feature (295)..(295): Xaa can be any naturally occurring amino acid
  misc_feature (343)..(343): Xaa can be any naturally occurring amino acid
  misc_feature (401)..(401): Xaa can be any naturally occurring amino acid
  misc_feature (426)..(426): Xaa can be any naturally occurring amino acid
  misc_feature (626)..(626): Xaa can be any naturally occurring amino acid
MSTDPKPQRKTKRNTNRRPQDVKFPGGGQIVGGVYLLPRRGPRLGVRAARKTSERSQPRGRRQPIPKDRRSPGKSWGKPGYPWPLYGNEGCGWAGWLLSPRGSRPTWGPTDPRHRSRNLGKVIDTITCGFADLMGYIPVVGAPVGGVARALAHGVRVLEDGINYATGNLPGCSFSIFLLALLSCLTVPASAVEVRNISSGYYXTNDCSNSSITWQLTNAVLHLPGCVPCENDNGTLRCWIQVTPNVAVKYRGALTHNLRTHVDMIVMAATVCSALYVGDVCGAVMIVSQAFIMSXERHNFTQECNCSIYQGHITGHRMAWDMMLGWSPTLTMILAYAARVPEXVLEVVFGGHWGVVFGLAYFSMQGAWAKVIAILLLVAGVDAGTYSSGATIGQGTRGLVXLFSAGPSQKISLINTNGSWHINRTXLNCNDSLQTGFIASLFYAKSFNSSGCPERLSSCRGLDDFRIGWGTLEYENNVTNDEDMRPYCWHYPPKPCGIVPARTVCGPVYCFTPSPVVVGTTDKQGVPTYSWGENETDVFLLNSTRPPQGAWFGCTWMNGTGFTKTCGAPPCRIRRDHTSTLDLLCPTDCFRKHPDTTYLKCGAGPWLTPKCLVDYPYRLWHYPCTXNFTIFKVRMYVGGVEHRFSAACNFTRGDRCRLEDRDRGQQSPLLHSTTEWAVLPCSFSDLPALSTGLLHLHQNIVDVQYLYGLSPAITKYIVKWEWVILLFLLLADARVCACLWMLIILGQAEA